(19) United States
(12) Patent Application Publication
(10) Pub. No.: US 2019/0263928 A1
(43) Pub. Date: Aug. 29, 2019

(54) ADAPTIVE CHIMERIC ANTIGEN RECEPTOR T-CELL DESIGN

(71) Applicant: Baylor College of Medicine, Houston, TX (US)

(72) Inventors: Norihiro Watanabe, Houston, TX (US); Malcolm K. Brenner, Bellaire, TX (US); Juan Fernando Valdes Vera, Houston, TX (US)

(21) Appl. No.: 16/334,717

(22) PCT Filed: Sep. 29, 2017
(86) PCT No.: PCT/US17/54604

§ 371 Date: Mar. 19, 2019

Related U.S. Application Data
(60) Provisional application No. 62/402,618, filed on Sep. 30, 2016.

(51) Int. Cl. C07K 16/30; C07K 14/725; C07K 14/705; A61P 35/00; C12N 5/0783; G01N 33/50; A61K 35/17

(52) U.S. Cl. CPC C07K 16/3069; C07K 14/7051; C07K 14/70521; A61P 35/00; C12N 5/0636; G01N 33/5047; C12N 2510/00; C07K 2317/622; C07K 2319/02; C07K 2319/03; C07K 2317/524; C07K 2317/526; A61K 35/17

(57) ABSTRACT

Embodiments of the disclosure include methods and compositions that allow for development of efficient chimeric antigen receptors (CARs) by selecting appropriate spacer content and/or length by balancing the effects of tonic signaling with the efficacy of antigen recognition for the spacer. In specific embodiments, the CH3 domain from IgG2 is utilized as a spacer. In specific embodiments, T cell metabolic activity is utilized as a measure of tonic signaling to facilitate determination of suitable CAR constructs. In other embodiments, cells bearing chimeric Fc receptor target molecules are utilized to target Fc gamma receptor (Fc [symbol] R)-bearing for the purpose of their destruction.

This application claims priority to U.S. Provisional Patent Application Ser. No. 62/402,618, filed Sep. 30, 2016, which is incorporated by reference herein in its entirety.

### STATEMENT REGARDING FEDERALLY SPONSORED RESEARCH OR DEVELOPMENT

This invention was made with government support under P50-CA 126752awarded by National Institutes of Health/National Cancer Institute. The government has certain rights in the invention.

### TECHNICAL FIELD

Embodiments of the disclosure encompass at least the fields of cell biology, molecular biology, immunology, and medicine.

### BACKGROUND

Recent advances in immunotherapy utilize adoptive transfer of human T lymphocytes engineered to express chimeric antigen receptors (CAR) that target surface molecules on tumor cells. CARs generally comprise an extracellular antigen-binding domain usually comprising a single chain variable fragment (scFv) of a monoclonal antibody (mAb) linked to one or more intracellular signaling components, including CD3zeta alone or in combination with one or more costimulatory domains. The focus of most research in CAR design has centered around identifying appropriate scFvs that, upon expression in T cells, confer recognition of malignant cells without unacceptable toxicity to normal tissues, although in some cases focus has included optimization of intracellular signaling modules to activate T-cell effector functions.

CD19-CAR-T-cell therapy has advanced furthest in clinical studies. In some cases, the CD19-CAR demonstrated antitumor activity in patients with advanced CLL, and this particular CAR comprised a short spacer sequence derived from CD8α that linked the scFv to the remainder of the CAR. In another CD19-CAR-T-cell therapy trial, antitumor efficacy and CD19-CAR-T-cell survival were not as successful, and in that particular CAR the spacer domain was longer and derived from the IgG1 hinge and Fc. One possible explanation is that different CAR constructs, including spacer length and/or composition, induce different degrees of tonic signaling in a T cell expressing the CAR; tonic signaling can lead to T cell anergy and loss of persistence.

The present disclosure provides a solution to identifying suitable spacer configurations for effective CAR construction and therapy.

### BRIEF SUMMARY

Embodiments of the disclosure include methods for optimizing the efficacy of engineered chimeric receptors to be employed in immune cells for immunotherapy. In specific embodiments, the methods occur in vitro and/or in vivo. The methods incorporate elements that have conflicting pressures to find a balance of optimized chimeric receptor components. In specific embodiments, the chimeric receptor comprises at least an antigen recognition domain and a spacer that separates the antigen recognition domain from another component of the receptor, such as at least one intracellular signaling domain.

In one aspect, provided herein are methods of evaluating one or more components of a chimeric antigen receptor (CAR) to determine the components' effects on an immune cell expressing the CAR. In particular, the methods provided herein comprise evaluating the effect of one or more components of a CAR on the anergy, persistence and/or apoptosis of an immune cell expressing the CAR, wherein the one or more components are a spacer, antigen recognition domain, exodomain comprising the antigen recognition domain or part thereof, a transmembrane domain, and/or an endodomain.

In one embodiment, methods of the disclosure secure an equilibrium between the efficacy of antigen recognition and cell activation by an engineered chimeric receptor in cells expressing the receptor with in vivo persistence for those cells. In particular aspects, method provides for optimization of the spacer length and/or content are optimized for utilization in an engineered chimeric receptor, e.g., a chimeric antigen receptor. The spacer length and/or content may be chosen for the specific purpose of evaluating one or more attributes of the receptor, in some cases. Any kind of attribute to show efficacy of the receptor may be utilized, but in certain cases one may evaluate a variety of in vitro and/or in vivo assays to obtain information.

In one embodiment, provided herein is a method of producing an engineered chimeric receptor having at least an antigen recognition domain and a spacer separating the antigen recognition domain from at least one other functional domain, wherein the spacer comprises an amino acid sequence, said method comprising the step of evaluating tonic signaling in cells expressing the receptor. In another specific embodiment, the method further comprises the step of evaluating antigen recognition for the receptor. In another specific embodiment, the method further comprises the step of evaluating anergy, persistence, or apoptosis of cells expressing the receptor. In specific embodiments, following any of such evaluating steps, at least part of the chimeric receptor is modified, such as the spacer, the antigen recognition domain, an exodomain comprising the antigen recognition domain or part thereof, a transmembrane domain, and/or an endodomain or part thereof.

In the context of the method provided herein, tonic signaling may be evaluated by one or more of the following: a) measuring metabolic activity of the cells; b) measuring one or more indicators of cell activation in the absence of stimulation by an antigen recognized by the receptor; c) measuring one or more phenotypical changes related to cell aging or cell senescence; d) determining cell cycle progression in the absence of antigenic stimulation; and e) measuring cell size of cells expressing the receptor compared to the size of unmodified cells.

Antigen recognition may be evaluated by one or more of the following: a) the efficacy of the binding of the antigen recognition domain to an antigen; b) an in vitro killing assay of one or more cells expressing the receptor; c) an in vivo assay measuring tumor size or burden following delivery of cells expressing the receptor; d) cytokine production of one or more cells expressing the receptor; e) the in vivo proliferation of one or more cells that express the receptor; f) antitumor activity of immune cells expressing the receptor; and g) measuring cell size of cells expressing the receptor compared to the size of unmodified cells. In specific embodiments, antigen recognition is evaluated by a) phenotype of cells expressing the receptor, b) growth pattern of cells expressing the receptor in the absence of the antigen in comparison to growth pattern of non-transduced cells; and/or c) the killing of target cells that express the antigen. In specific cases, when the T cell phenotype comprises a high content of naïve and central memory cells among cells expressing the receptor, the antigen recognition is effective. In some cases, when cells expressing the receptor have a high content of CCR7+ after about two weeks in the absence of the antigen, the antigen recognition is effective. In particular cases, when at least 30% of cells expressing the receptor are CCR7+ after about two weeks in absence of the antigen, the antigen recognition is effective. Among cells expressing the receptor, when the amount of CCR7+ cells is similar to the number of CCR7+ non-transduced cells under the same culture conditions, the antigen recognition is effective, in at least some cases. In specific embodiments, when the growth pattern of cells expressing the receptor is similar to non-transduced T cells in the absence of the antigen, the antigen recognition is effective.

When metabolic activity is assessed, the metabolic activity may be measured within 2 to 3 days after transduction of the cells with a polynucleotide encoding the receptor. In specific cases, the metabolic activity is determined by the level of glucose produced by the cell, the level of lactate produced by the cell, or a ratio thereof In specific embodiments, one or more indicators of cell activation comprise the level of CD25, CD69, or both, in the cells, and the one or more indicators of cell activation may comprise the level of one or more cytokines produced by the cells, such as interferon gamma, TNF, IL2, INFb, GMCSF, perforin, IL13, IL4, TGFb, or a combination thereof. In some cases, when the receptor comprises the CD3 zeta chain, the one or more indicators of cell activation comprises the phosphorylation of the CD3 zeta chain in the absence of antigenic stimulation. In particular cases, cytokine production comprises production of interferon gamma, IL2, TNF, INFb, GMCSF, perforin, IL13, IL4, TGFb, or a combination thereof. In specific aspects, the one or more indicators of cell activation comprise the level of CD25, CD69, 41BB, CD71, CD40, HLADR alone or in combination.

In some embodiments, the spacer length and/or content is selected for the purpose of evaluating for suitability for use an engineered receptor such as a CAR.

In certain embodiments, when an epitope on an antigen to which the receptor binds is proximal to the cell membrane, a spacer that is >150 amino acids is selected for the CAR. In other cases, when an epitope on an antigen to which the receptor binds is exposed or distal to the cell membrane, a spacer that is <50 amino acids is selected. The spacer may be derived from IgG2 and may comprise CH2 and CH3 from IgG2, in certain cases. In specific cases, the spacer comprises the hinge from IgG2. The spacer may comprise CH3 from IgG2. In certain cases, the spacer lacks CH2 from IgG2. The spacer may comprise one or more modifications to reduce binding of the spacer to an Fcγ receptor.

Particular embodiments of the method employ assessment of epitope proximity to the cell surface such that one can characterize the spacer length and the location of the epitope on the antigen.

In another aspect, provided herein is a polynucleotide encoding the engineered receptor produced by the method encompassed by the disclosure, and the polynucleotide may be comprised in a vector, such as one comprised in a cell, including an immune cell, such as a T lymphocyte, NK cell, or NKT cell. In another embodiment, there is a chimeric antigen receptor encoded by a polynucleotide encompassed by the disclosure and/or produced by a method of the disclosure.

In another aspect, provided herein are chimeric antigen receptors produced by any of the methods provided herein.

In another aspect, provided herein is a pharmaceutical composition comprising a chimeric antigen receptor encompassed by the disclosure.

In another aspect, provided herein is a cell expressing a polynucleotide or expressing a receptor encompassed by the disclosure.

In another aspect, provided herein is a method of targeting a Fc-gamma receptor (FcγR)-bearing cell, comprising the step of contacting the FcγR-bearing cell with an immune cell that expresses a chimeric Fc receptor target molecule that comprises one or more FcγR-binding domains of an IgG Fc domain, wherein the contacting is deliberately performed to target the FcγR-bearing cell. In a specific embodiment, the FcγR-binding domain comprises the CH2CH3 region, the CH2 region, and/or the CH3 region of an IgG. In some cases, the CH2CH3 region, the CH2 region, and/or the CH3 region is from IgG1, IgG2, or IgG4. The chimeric Fc receptor target molecule may further comprise CD3 zeta-chain of the TCR/CD3 complex and the FcγR-bearing cell is killed. In specific embodiments, the chimeric Fc receptor target molecule comprises or further comprises an scFv. In specific cases, the chimeric Fc receptor target molecule lacks the CD3 zeta-chain of the TCR/CD3 complex. The chimeric Fc receptor target molecule may comprise one or more costimulatory domains, such as CD28, OX40, 4-1BB, ICOS, CD27, CD95, CD43, KLRG1, CD4OL, CD137, CD137L, CD134, or a combination thereof. The immune cell may be a T cell, NK cell, NKT cell, B cells, monocytes, macrophages, or dendritic cells. In specific cases, the FcγR-bearing cell is a monocyte, macrophage, dendritic cell, neutrophil, eosinophils, platelets (RIIa), B cells (RIIIb), or NK (RIIc). The method may occur in vivo in an individual that has a medical condition with chronic inflammation as a symptom, such as chronic inflammation is arthritis, multiple sclerosis, diabetic ulcers, atherosclerosis, asthma, sepsis, cardiovascular disease, or Alzheimer's Disease. In specific embodiments, said targeting occurs in vivo in an individual that has cancer of any kind (including at least lung cancer), arthritis, multiple sclerosis, diabetic ulcers, atherosclerosis, asthma, sepsis, cardiovascular disease, or Alzheimer's Disease.

In another aspect, provided herein is a method of treating an individual having cancer, comprising administering to the individual a therapeutically effective amount of a chimeric antigen receptor or a pharmaceutical composition encompassed by the disclosure, wherein the cancer expresses a tumor-associated antigen or tumor-specific antigen, and the chimeric antigen receptor is targeted to the tumor-associated antigen or tumor-specific antigen. The cancer may be primary, metastatic, refractory, or sensitive to one or more agents, and the cancer may be of any tissue origin, including lung, breast, brain, prostate, colon, liver, kidney, skin, bone, testicular, ovarian, cervical, rectal, head and neck, thyroid, gall bladder, stomach, pituitary gland, endometrial, blood, and so forth.

In another aspect, provided herein is a method of selecting a chimeric antigen receptor having a spacer between an antigen recognition domain and a transmembrane domain, comprising the steps of (a) expressing a first chimeric antigen receptor in a type of immune cell and determining a first level of tonic signaling in the immune cell; (b) subsequently expressing a second chimeric antigen receptor having a longer or shorter spacer; (c) expressing the chimeric antigen receptor having said longer or shorter spacer in said type of immune cell, and determining a second level of tonic signaling in the immune cell; wherein if said second level is lower than said first level, said second chimeric antigen receptor is selected, and if said first level is lower than said first level, said first chimeric antigen receptor is selected. In specific embodiments, the method comprises repeating steps (a)-(c) for a plurality of times with chimeric antigen receptors having spacers of a different length for each of said plurality of times, and selecting said chimeric antigen receptor that is expressed by the immune cell determined to have the least tonic signaling.

In another aspect, provided herein is a method of designing an engineered chimeric receptor having a spacer and an antigen recognition domain, comprising the steps of: a) evaluating tonic signaling in cells expressing the receptor; and optionally b) evaluating efficacy of the receptor and/or antigen recognition and/or antitumor activity of immune cells expressing the receptor; and selecting a suitable spacer length based on said evaluating steps. In some cases, the evaluating in step a) comprises one or more of the following: 1) measuring metabolic activity of the cells; 2) measuring one or more indicators of cell activation in the absence of stimulation by an antigen recognized by the receptor; 3) measuring one or more phenotypical changes related to cell aging; 4) determining cell cycle progression in the absence of antigenic stimulation; and/or 5) measuring cell size. In certain embodiments, the evaluating in step b) comprises one or more of the following: 1) the efficacy of the binding of the antigen recognition domain to an antigen; 2) an in vitro killing assay of one or more cells expressing the receptor; 3) an in vivo assay measuring tumor size or burden following delivery of cells expressing the receptor; 4) cytokine production of one or more cells expressing the receptor; 5) the in vivo proliferation of one or more cells that express the receptor; and/or 6) measuring cell size.

In some aspects, tonic signaling is evaluated by one or more of the following: a) measuring metabolic activity of the cells in the absence of antigenic stimulation and compared to unmodified cells and/or a control vector without tonic signaling; b) measuring one or more indicators of cell activation in the absence of antigenic stimulation and compared to unmodified cells and/or a control vector without tonic signaling; c) measuring one or more phenotypical changes related to cell aging or cell senescence in the absence of antigenic stimulation and compared to unmodified cell and/or a control vector without tonic signaling; d) determining cell cycle progression in the absence of antigenic stimulation and compared to unmodified cells and/or a control vector without tonic signaling; e) measuring cell size of cells expressing the receptor in the absence of antigenic stimulation and compared to unmodified cells and/or a control vector without tonic signaling; and f) measuring the cytokine production of cells in the absence of antigenic stimulation and compared to unmodified cells and/or a control vector without tonic signaling.

In some aspects, antigen recognition by said antigen recognition domain is evaluated by one or more of the following: a) the efficacy of the binding of the antigen recognition domain to an antigen; b) an in vitro killing assay of one or more cells expressing the receptor; c) an in vivo assay measuring tumor size or burden following delivery of cells expressing the receptor; d) cytokine production of one or more cells expressing the receptor; e) the in vivo proliferation of one or more cells that express the receptor; and f) antitumor activity of immune cells expressing the receptor. In specific embodiments, the tonic signaling is evaluated by a) phenotype of cells expressing the receptor, b) growth pattern of cells expressing the receptor in the absence of the antigen in comparison to growth pattern of non-transduced cells and/or a control vector without tonic signaling. In some cases, when the T cell phenotype comprises a similar content (for example, within 10%; higher may be >10% and lower may be <10%) of naive and central memory cells among cells expressing the receptor compare to non-transduced cells and/or a control vector, it is considered to have a low tonic signal. In some aspects, when cells expressing the receptor have a high content of CCR7+ after about two weeks in the absence of the antigen, the cells are predicted to have low tonic signaling. In some cases, when at least 30% of cells expressing the receptor are CCR7+ after about two weeks in absence of the antigen, the cells are predicted to have low tonic signaling. In specific cases, among cells expressing the receptor, when the amount of CCR7+ cells is similar to the number of CCR7+ non-transduced cells under the same culture conditions, and/or control construct under the same culture conditions, the cells are predicted to have low tonic signaling.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1G—CAR-PSCA T cells have in vitro antitumor activity but fail to exert an in vivo antitumor response in a subcutaneous tumor model—(1A) Representation of prototype 2G.CAR.PSCA construct (P1.CAR)—vector map and schematic. (1B) P1.CAR expression on primary T cells. CAR expression was detected by anti-F(ab′)2 antibody conjugated with AlexaFluor 647 (open: NT cells, filled: CAR T cells). The number indicates mean±S.E (n=8). (1C) The cytolytic function of P1.CAR T cells in a 4 hr ⁵¹Cr-release assay against PSCA⁺ targets (K562-PSCA and Capan-1) and PSCA⁻ targets (K562 and 293T cells) (open: NT cells, filled: P1.CAR). Data represents mean±S.E (n=5). Significance was determined by two-way ANOVA. *p<0.05 compared to NT cells. (1D) Capan-1 tumor growth in vivo. Graph shows the tumor volume in NSG mice engrafted with Capan-1 s.c. and treated with PBS (open) and P1.CAR T cells (filled). (1E) In vivo T cell distribution were detected by bioluminescence imaging. (1F) Fcγ receptor types I, II, and III on monocytes, macrophages and NK cells were analyzed by FACS (black: isotype, red: FcγR). (1G) Representative FACS plot from 6 independent coculture experiments is shown. Cell number was counted by FACS with counting beads in the cocultures either NT cells (left) or P1.CAR (right) with monocytes, macrophages or NK cells on Day 0 and Day 3.

FIGS. 2A-2F—Modification of CH2CH3 spacer results in improved T cell localization to the tumor site—(2A) Representation of modified 2G.CAR.PSCA constructs (M1.CAR and M2.CAR)—vector map and schematic. (2B) M1.CAR and M2.CAR expression on primary T cells. CAR expression was detected by anti-F(ab′)2 antibody conjugated with AlexaFluor 647 (open: NT cells, filled: CAR T cells). The number indicates mean±S.E (n=8). (2C) The cytolytic function of M1.CAR and M2.CAR T cells in a 4 hr ⁵¹Cr-release assay against PSCA⁺ targets (K562-PSCA and Capan-1) and PSCA³¹ targets (K562 and 293T cells) (open: NT cells, black: P1.CAR, blue: M1.CAR, red: M2.CAR). Data represents mean±S.E (n=5). Significance was determined by two-way ANOVA. *p<0.05 compared to NT cells. (2D) Representative FACS plot from 6 independent coculture experiments is shown. Three days after coculture of T cells with macrophage (left) or monocyte (right), cell number was counted by FACS with counting beads. Bar graphs represent total cell number count (black: T cells, white: macrophages or monocytes) with mean±S.E (n=6). Significance was determined by unpaired 2-tailed t-test. *p<0.05 compared to NT cell cocultures. (2E) In vivo T cell distribution were detected by bioluminescence imaging. (2F) Capan-1 tumor growth in vivo. Graph shows the tumor volume in NSG mice engrafted with Capan-1 s.c. and treated with PBS (open), P1.CAR (black), M1.CAR (blue) and M2.CAR T cells (red). Significance was determined by two-way ANOVA. *p<0.05.

FIGS. 3A-3E—CAR T cells appear to have accelerated cell senescence—(3A) The cytolytic function of P1.CAR T cells cultured for 10, 20 and 30 days after transduction. A 4 hr ⁵¹Cr-release assay was performed at a 40:1 ratio against 293T (PSCA⁻ targets, open) and DU145 (PSCA⁺ targets, filled). The bar graph represents mean±S.E (n=3). Significance was determined by one-way ANOVA for DU145. n.s: not significant. (3B) The number of T cells (open) and tumor cells (filled) after 6 days in a coculture experiment was determined by FACS with counting beads. P1.CAR T cells, which were cultured in in vitro for 10, 20 and 30 days after transduction, were cocultured with DU145. Graph represents mean±S.E (n=4). Significance was determined by one-way ANOVA with Bonferroni's multiple comparisons test. *p<0.05 compared to Day 10 T cell cocultures. (3C) Volcano plot of microarray analysis with common differentially expressed genes in T cells cultured for 20 days versus 10 days after transduction from 3 independent donors. (3D) Fold change of gene expression of Day20 and Day30 T cells compared to Day10 T cells. All listed genes were significantly upregulated or downregulated determined by FDR corrected ANOVA analysis (p<0.05). (3E) Surface phenotypes of CD8⁺ T cells were analyzed on Day10, 20 and 30 after transduction. Representative data is shown by FACS plot—CCR7/CD45R0 (left) and CD27/CD28 (right). The pie chart represents mean±S.E (n=6) on Day30. Significance was determined by unpaired two-tailed t-test. *p<0.05 compared to NT cells. Tnaive: naïve, Tcm: central memory, Tem: effector memory, Temra: terminally differentiated.

FIGS. 4A-4G—Tonic signaling is responsible for accelerated T cell aging—(4A) Representation of control CAR construct (ΔCAR)—vector map and schematic. (4B) ΔCAR expression on primary T cells. CAR expression was detected by anti-F(ab′)2 antibody conjugated with AlexaFluor 647 (open: NT cells, filled: CAR T cells). The number indicates mean±S.E (n=7). (4C) Representative histogram of phospho-CD247 (CD3z) staining for different CAR T cells are shown (n=6) (open: NT cells, gray: ΔCAR, black: P1.CAR, blue: M1.CAR, red: M2.CAR). (4D) Representative histogram of CD25 expression on CD8⁺ T cells (left panel) and summarized for 6 donors (right panel, mean±S.E). (4E) Representative FACS plot for cell cycle analysis. Cells were stained with 7AAD and Ki-67 on day 20 after transduction. The pie chart represents mean±S.E (n=3). Significance was determined by unpaired two-tailed t-test. *p<0.05 compared to NT cells. (4F) Fold-expansion of in vitro cultured cells as measured by manual hemocytometer using trypan blue (open: NT cells, gray: ΔCAR, black: P1.CAR, blue: M1.CAR, red: M2.CAR). (4G) Spontaneous cytokine release from different CAR T cells. The levels of GM-CSF, TNFα and IFNγ in the supernatant were measured by Luminex assay in the absence of antigen stimulation. Bar graph represents mean±S.D (n=3) (open: NT cells, gray: ΔCAR, black: P1.CAR, blue: M1.CAR, red: M2.CAR). Significance was determined by unpaired two-tailed t-test. *p<0.05 compared to NT cells.

FIGS. 5A-5H—CH2CH3 spacer present within the CAR is responsible for tonic T cell signaling—(5A) Representation of X2.CAR construct (deleted CH2CH3 sequence)—vector map and schematic. (5B) X2.CAR expression on primary T cells (open: NT cells, filled: CAR T cells). The number indicates mean±S.E (n=8). (5C) The representative histogram of CD25 expression on CD8⁺ T cells (black line: CD25 for NT cells, red: CD25 for CAR T cells, gray and light red: isotype for NT cells and CAR T cells; respectively). Line graph shows the percentage of CD25 positive cells in the CD8⁺ T cell over time with mean±S.E (n=6) (gray: ΔCAR, red: M2.CAR, green: X2.CAR). (5D) Representative FACS plot for cell cycle analysis. The pie chart represents mean±S.E (n=3). Significance was determined by unpaired two-tailed t-test. *p<0.05 compared to M2.CAR. (5E) Fold-expansion of in vitro cultured cells (gray: ΔCAR, red: M2.CAR, green: X2.CAR). (5F) Surface phenotypes of CD8⁺ T cells were analyzed on Day10, 20 and 30 after transduction. Representative data is shown by FACS plot—CCR7/CD45R0 (left) and CD27/CD28 (right). The pie chart represents mean ±S.E (n=6) on Day30. Significance was determined by unpaired two-tailed t-test. *p<0.05 compared to M2.CAR. (5G) Spontaneous cytokine release from different CAR T cells. Bar graph represents mean±S.D (n=3) (gray: ACAR, red: M2.CAR, green: X2.CAR). Significance was determined by unpaired two-tailed t-test. *p<0.05 compared to M2.CAR. (5H) The cytolytic function of CAR T cells in a 4 hr ⁵¹Cr-release assay against PSCA^{bright} targets (K562-PSCA and Capan-1), PSCA^{dim} targets (DU145 and CFPAC-1) and PSCA⁻ targets (K562 and 293T cells) (open: NT cells, red: M2.CAR, green: X2.CAR). Data represents mean±S.E (n=5). Significance was determined by two-way ANOVA. *p<0.05 compared to NT cells.

FIGS. 6A-6H—Incorporation of CH3 as a spacer can decrease cell aging and restore killing abilities—(6A) Representation of X₃2.CAR construct (incorporated CH3 sequence)—vector map and schematic. (6B) X32.CAR expression on primary T cells (open: NT cells, filled: CAR T cells). The number indicates mean±S.E (n=8). (6C) The cytolytic function of CAR T cells in a 4 hr ⁵¹Cr-release assay against PSCA^{bright} targets (K562-PSCA and Capan-1), PSCA^{dim} targets (DU145 and CFPAC-1) and PSCA⁻ targets (K562 and 293T cells) (open: NT cells, red: M2.CAR, green: X2.CAR, purple: X₃2.CAR). Data represents mean±S.E (n=5). Significance was determined by two-way ANOVA. *p<0.05 compared to NT cells. (6D) Line graph shows the percentage of CD25 positive cells in the CD8⁺ T cell (top) and CD4⁺ T cell (bottom) over time with mean±S.E (n=6) (gray: ΔCAR, red: M2.CAR, green: X2.CAR, purple: X32.CAR). (6E) The pie chart represents the surface phenotype of CD8⁺ T cells (top) and CD4⁺ T cells (bottom) cultured for 30 days after transduction with mean±S.E (n=6)—CCR7/CD45R0(left) and CD27/CD28 (right). Significance was determined by unpaired two-tailed t-test. *p<0.05 compared to M2.CAR. (6F) Representative FACS plot for cell cycle analysis. The pie chart represents mean±S.E (n=3). Significance was determined by unpaired two-tailed t-test. *p<0.05 compared to M2.CAR. (6G) Fold-expansion of in vitro cultured cells (gray: ACAR, red: M2.CAR, green: X2.CAR, purple: X32.CAR). (61) Spontaneous cytokine release from different CAR T cells. Bar graph represents mean±S.D (n=3) (gray: ACAR, red: M2.CAR, green: X2.CAR, purple: X32.CAR). Significance was determined by unpaired two-tailed t-test. *p<0.05 compared to M2.CAR.

FIGS. 7A-7E—In vivo CAR T cell function is enhanced using an adaptive CAR design—(7A) In vivo T cell distribution were detected by bioluminescence imaging. (7B) Total bioluminescence at tumor site over time after T cell injection with mean±S.E (n=5). (7C) Total bioluminescence from mice on day 35 after T cell injection with mean±S.E (n=5). (7D) Capan-1 tumor growth in vivo. Graph shows the tumor volume in NSG mice engrafted with Capan-1 s.c. and treated with PBS (open), P1.CAR (black), M1.CAR (blue), M2.CAR (red), X2.CAR (green) and X32.CAR T cells (purple). Significance was determined by two-way ANOVA. *p<0.05. (7E) The overall survival of mice treated with the various CAR T cells (open: PBS, black: P1.CAR, blue: M1.CAR, red: M2.CAR, green: X2.CAR, purple: X32.CAR). Significance was determined by log-rank test. *p<0.05.

FIGS. 8A-8B—Cell senescence of CD4⁺ T cells - Surface phenotype of CD4⁺ T cells were analyzed on Day10, Day20 and Day30 after transduction by FACS. Representative data is shown by FACS plot at different time points and the pie chart represents mean±S.E (n=6) on Day30. Representative data is shown by FACS plot—CCR7/CD45RO (8A) and CD27/CD28 (8B). Significance was determined by unpaired two-tailed t-test. *p<0.05 compared to NT cells.

FIGS. 9A-9B—Activation status of CD4⁺ T cells—CD25 expression was tracked for CD4⁺ T cells on Day10, Day20 and Day30 after transduction by FACS. (9A) Representative histogram is shown (black line: CD25 for NT cells, blue: CD25 for CAR T cells, gray and light blue: isotype for NT cells and CAR T cells; respectively). (9B) Line graph shows the percentage of CD25 positive cells in the CD4⁺ T cell over time with mean±S.E (n=6) (open: NT cells, gray: ACAR, black: P1.CAR, blue: M1.CAR, red: M2.CAR).

FIGS. 10A-10D—Coculture experiments with FcγR-expressing cells and phenotype of X2.CAR T cells—(10A) Representative FACS plot from 3 independent coculture experiments is shown. Three days after coculture of T cells with macrophage (left) or monocyte (right), cell number was counted by FACS with counting beads. Bar graphs represent total cell number count (black: T cells, white: macrophages or monocytes) with mean±S.E (n=3). (10B) CD25 expression on CD4⁺ T cells is shown by the representative histogram at different time points (left) and line graph (right) with mean±S.E (n=6). For the histogram, black line: CD25 for NT cells, blue: CD25 for CAR T cells, gray and light blue: isotype for NT cells and CAR T cells; respectively. For the line graph, gray: ACAR, red: M2.CAR, green: X2.CAR. (10C) Surface phenotype of CD4⁺ T cells were analyzed on Day10, Day20 and Day30 after transduction by FACS. Representative data is shown by FACS plot and the pie chart represents mean±S.E (n=6) on Day30. Significance was determined by unpaired two-tailed t-test. *p<0.05 compared to M2.CAR. (10D) PSCA expression for different cells lines is shown.

FIG. 11—Fc-FcγR interaction of X₃2.CAR T cells—Representative FACS plot from 3 independent coculture experiments is shown. Three days after co-culture of T cells with macrophages (left) or monocytes (right), cell number was counted by FACS with counting beads. Bar graphs show each cell number count (black: T cells, white: macrophages or monocytes) with mean±S.E (n=3).

FIGS. 12A-12B—T cell migration to the lung and PSCA expression on tumor cells from in vivo—(12A) In vivo T cell distributions are evaluated on Day3 after T cell injection. Representative mice images are shown in the left. Bar graph represents bioluminescence signal at the lung with mean±S.E (n=5). Significance was determined by unpaired two-tailed t-test. *p<0.05 compared to P1.CAR. (12B) PSCA expression on tumor cells are analyzed by FACS (black: isotype, red: PSCA). Bar graph represents relative MFI of PSCA expression with mean±S.E (n=2-5). Significance was determined by unpaired two-tailed t-test. *p<0.05 compared to PBS treated.

FIGS. 13A-13F—Prediction of the tonic signaling—(13A) Fold expansion at different period is shown in the line graphs with mean±S.E (n=6). Cell number counts at different time points were evaluated by trypan blue and fold expansion was calculated. (13B) Fold expansion on Day3 after transduction is shown in line graphs with mean±S.E (n=5) (black: ΔCAR, Red: CAR T cells). (13C) Cell viability on Day3 after transduction was evaluated by FACS based on FSC and SSC. Bar graph represent mean±S.E (n=14). (13D) Cell size on Day3 after transduction was evaluated by FACS based on FSC. Representative histograms are shown in left (Black: ΔCAR, Red: CAR T cells) and the bar graph represents mean±S.E (n=14). (13E) CD25 expression on Day3 after transduction was analyzed by FACS in each CD3⁺CD4⁺ and CD3⁺CD8⁺ fraction. Representative histograms are shown in left (black: CD25 for ΔCAR, red/blue: CD25 for CAR T cells, gray and light red/blue: isotype for NT cells and CAR T cells; respectively). Bar graph represents relative MFI of CD25 expression with mean±S.E (n=5). (13F) Glucose concentration (mg/dL) and Lactate concentration (mM) in the supernatant was measured on Day3 after transduction (blue: glucose, red: lactate). Each concentration was normalized by cell number (left) and the Glucose/Lactate ratio was calculated (right). Both bar graph represent mean±S.E (n=5). Significance was determined by unpaired two-tailed t-test. *p<0.05 compared to ΔCAR.

FIG. 14—PD1 expression on various CAR modified T cells—PD1 expression was analyzed on T cells cultured for 10 days after transduction. Upper panel shows a representative histogram while bottom graph illustrates summary data (mean±S.E, n=3).

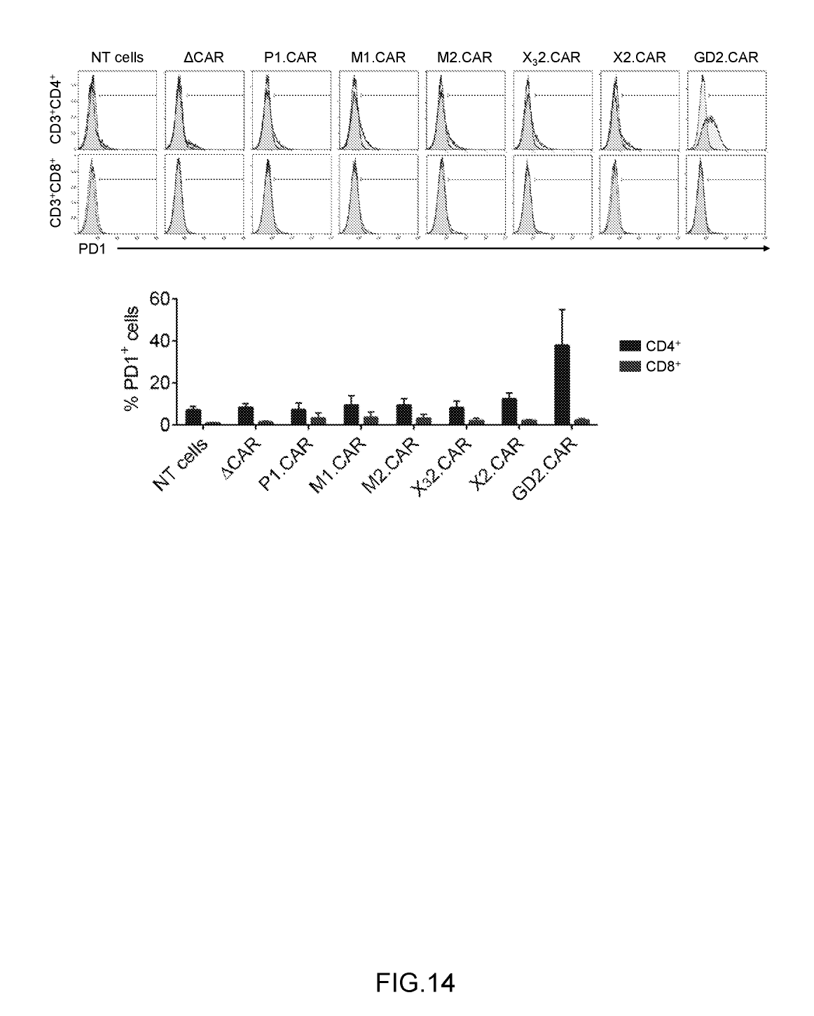

FIG. 15—The figure is an illustration of how lactate concentration can be plotted over time to determine a baseline lactate production in a controlled vector devoid of tonic signal such as: (i) a CAR without a signaling domain, (ii) a fluorescent molecule such as GFP, (iii) a truncated marker such as CD19 or CD24, (iv) an empty vector, and (v) non-transduced cells.

FIG. 16—Once the lactate concentration baseline has been identified (T cell culture condition known to not contain levels of tonic signaling). This can be used to evaluate the tonic signaling among different constructs and establish a hierarchy by identifying the one with the greatest tonic signaling as the configuration furthest away from the baseline.

FIG. 17—In this example, the lactate concentration is illustrated over time for Construct A vs. the Control vector that does not contain tonic signaling.

FIG. 18—In this example, the lactate concentration is illustrated over time for Construct B vs. the Control vector that does not contain tonic signaling.

FIG. 19—In this example, the lactate concentration is illustrated over time for Construct C vs. the Control vector that does not contain tonic signaling.

FIG. 20—In this example, the lactate concentration is illustrated over time for Construct D vs. the Control vector that does not contain tonic signaling.

FIG. 21—In this example, the lactate concentration is illustrated over time of multiple constructs vs. the Control vector that does not contain tonic signaling.

FIG. 22—By comparing the lactate concentration among these different constructs, one can observe Construct C as closest to the baseline, indicating that this one will be the lowest with tonic signaling, followed by Construct D. This comparison can then be used to establish a hierarchy of tonic signaling where the most favorable configuration will be identified as the one closest to the baseline.

FIG. 23—The following is an illustration of how glucose concentration can be plotted over time to determine a baseline glucose production in a controlled vector devoid of tonic signal such as: (i) a CAR without a signaling domain, (ii) a fluorescent molecule such as GFP, (iii) a truncated marker such as CD19 or CD24, (iv) an empty vector, and (v) non-transduced cells.

FIG. 24—Once the glucose concentration baseline has been identified (by a T cell culture condition known to not contain levels of tonic signaling), one can then evaluate the tonic signaling among different constructs and establish a hierarchy by identifying the one with the greatest tonic signaling as the configuration furthest away from the baseline.

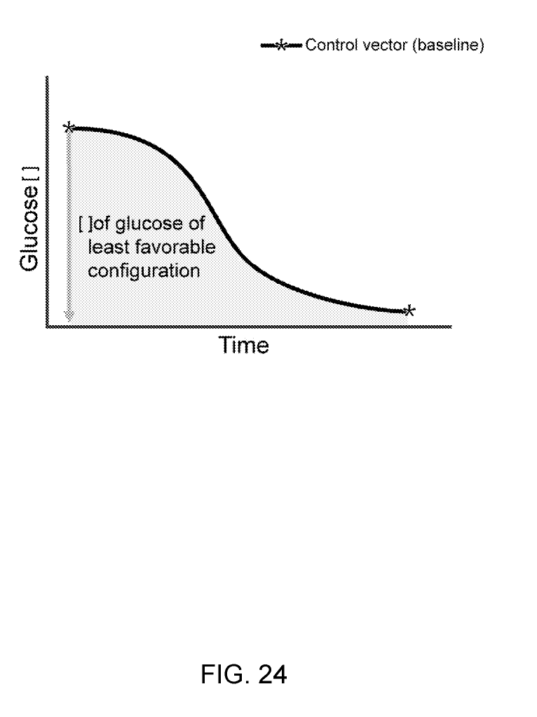

FIG. 25—In this example, glucose concentration is illustrated over time for Construct A vs. the Control vector that does not contain tonic signaling.

FIG. 26—In this example, glucose concentration is illustrated over time for Construct B vs. the Control vector that does not contain tonic signaling.

FIG. 27—In this example, glucose concentration is illustrated over time for Construct C vs. the Control vector that does not contain tonic signaling.

FIG. 28—In this example, glucose concentration is illustrated over time for Construct D vs. the Control vector that does not contain tonic signaling.

FIG. 29—In this example, glucose concentration is illustrated over time of multiple constructs vs. the Control vector that does not contain tonic signaling.

FIG. 30—By comparing the glucose concentration among these different constructs, one can observe Construct C as closest to the baseline, indicating that this one will be the lowest with tonic signaling, followed by Construct D. This comparison can then be used to establish a hierarchy of tonic signaling where the most favorable configuration will be identified as the one closest to the baseline.

FIG. 31—In this case, Construct A illustrates the pattern of glucose consumption of T cells expressing a truncated CAR-PSCA that lacks the signaling endodomain (glucose consumption baseline).

FIG. 32—This example illustrates how the baseline of glucose consumption can be obtained by using a CAR-lacking endodomain (Construct A), and comparing this with T cells that are non-transduced (Construct B). Therefore, either Control A or B can be used to establish the baseline.

FIG. 33—This figure illustrates the glucose concentration of the control construct A and the glucose concentration of Test construct A when measured at Day 3 of the culture.

FIG. 34—This figure illustrates the glucose concentration of the control construct A and the glucose concentration of Test construct B when measured at Day 3 of the culture.

FIG. 35—This figure illustrates the glucose concentration of the control construct A and the glucose concentration of Test construct C when measured at Day 3 of the culture.

FIG. 36—This figure illustrates the glucose concentration of the control construct A and the glucose concentration of Test construct D when measured at Day 3 of the culture.

FIG. 37—The glucose concentration of multiple test conditions can then be compared as long as the same time set has been acquired for all test conditions. This example also illustrates how a single time assessment is sufficient to make this comparison. Therefore, construct D has the lowest tonic signaling as this is closest to the baseline.

FIG. 38—Based on the difference in glucose concentration, one can establish a hierarchy where in this case, the most favorable configuration is the one with the lowest tonic signaling.

FIG. 39—In this case, Construct A illustrates the pattern of lactate consumption of T cells expressing a truncated CAR-PSCA that lacks the signaling endodomain (lactate consumption baseline).

FIG. 40—This example illustrates how the baseline of lactate consumption can be obtained by using a CAR-lacking endodomain (Construct A), and comparing this with T cells that are non-transduced (Construct B). Therefore, either Control A or B can be used to establish the baseline.

FIG. 41—This figure illustrates the lactate concentration of the control construct A and the lactate concentration of Test construct A when measured at Day 3 of the culture.

FIG. 42—This figure illustrates the lactate concentration of the control construct A and the lactate concentration of Test construct B when measured at Day 3 of the culture.

FIG. 43—This figure illustrates the lactate concentration of the control construct A and the lactate concentration of Test construct C when measured at Day 3 of the culture.

FIG. 44—This figure illustrates the lactate concentration of the control construct A and the lactate concentration of Test construct D when measured at Day 3 of the culture.

FIG. 45—The lactate concentration of multiple test conditions can then be compared as long as the same time set has been acquired for all test conditions. This example also illustrates how a single time assessment is sufficient to make this comparison. Therefore, construct D has the lowest tonic signaling as this is closest to the baseline.

FIG. 46—Based on the difference in glucose and lactate concentration, one can establish a hierarchy where in this case, the most favorable configuration is the one with the lowest tonic signaling.

FIG. 47—Therefore, the concentration of glucose and lactate collected from the media of T cells expression these different constructs can be used to establish a hierarchy of tonic signaling.

FIG. 48—This figure illustrates an example of a vector map of CAR constructs containing various spacer length.

FIG. 49—This figure illustrates the CAR expression of T cells after retroviral transduction. The upper panel shows the staining used in an anti-IgG antibody, as expected the “short IgG2 CAR” is not stained as this molecule does not contain CH2CH3. In the lower panel, this illustrates the CAR expression using an anti-F(ab′)2 antibody, in this condition all the molecules are detected.

FIG. 50—This figure illustrates the killing of CARs with different lengths of spacers.

FIG. 51—This figure illustrates the killing of CARs with different lengths of spacers. Note: when targeting tumor cells that express intermediate levels of antigen expression the CAR with the short spacer resulted in reduced antigen recognition properties.

FIG. 52—This figure illustrates the killing of CARs with different lengths of spacers.

FIG. 53—This figure illustrates the killing of CARs with different lengths of spacers. Note: when targeting tumor cells that express low levels of antigen expression the CAR with the short and intermediate spacer resulted in reduced antigen recognition properties.

FIG. 54—This figure illustrates the killing of CARs with different lengths of spacers.

FIG. 55—This figure illustrates the killing of CARs with different lengths of spacers. Note: when targeting tumor cells that express high levels of antigen expression the CAR with a long, intermediate, or short spacer resulted in similar killing properties.

FIG. 56—This figure illustrates the antigen expression (PSCA) on two different cancer cells lines.

FIG. 57—This figure shows the memory profile of T cells transduced with different CAR constructs after culture for 20 days in media with IL2 in absence of antigen stimulation.

FIG. 58—This figure illustrates the naive phenotype versus the central memory phenotype of CD4 T cells, transduced with different CAR constructs, at 10 days of culture.

FIG. 59—This figure illustrates the naive phenotype versus the central memory phenotype of CD4 T cells, transduced with different CAR constructs, at 20 days of culture.

FIG. 60—This figure illustrates the naive phenotype versus the central memory phenotype of CD4 T cells, transduced with different CAR constructs, at 30 days of culture.

FIG. 61—This figure illustrates the naive phenotype versus the central memory phenotype of CD8 T cells, transduced with different CAR constructs, at 30 days of culture.

FIG. 62—This figure shows the differences of co-stimulatory molecules (CD27/CD28) profile of T cells transduced with different CAR constructs after culture for 20 days in media with IL2 in absence of antigen stimulation.

FIG. 63—This figure illustrates the double positive CD27/CD28 population and single CD28 population on CD4 T cells transduced on different CAR configurations at Day 10 of culture.

FIG. 64—This figure illustrates the double positive CD27/CD28 population and single CD28 population on CD4 T cells transduced on different CAR configurations at Day 20 of culture.

FIG. 65—This figure illustrates the double positive CD27/CD28 population and single CD28 population on CD4 T cells transduced on different CAR configurations at Day 30 of culture.

FIG. 66—This figure illustrates the current knowledge based on what is known in the art. In this schematic representation, the X-axis represents the killing ability of T cells (where “killing” refers to shorter in vitro interaction as illustrated by a 4 hour chromium release assay) this can be considered as a magnitude of antigen recognition. The Y-axis represents the length of the CAR spacer.

FIG. 67—This figure illustrates the current knowledge based on what is known in the art. In this schematic representation, the X-axis represents the killing ability of T cells (where “killing” refers to shorter in vitro interaction as illustrated by a 4 hour chromium release assay) this can be considered as a magnitude of antigen recognition. The Y-axis represents the length of the CAR spacer.

FIG. 68—This figure represents an aspect previously unknown in the field. The inventors' work, as shown in this figure, describes a direct correlation between the CAR spacer and tonic signaling.

FIG. 69—Consideration of two opposing components: (i) antigen recognition (previously known to be related with the length of the CAR) and (ii) tonic signaling, one can see that the most favorable configuration regarding the length of the CAR is one that has both of these components.

FIG. 70—Traditionally, CARs function by the recognition of the antigen that is expressed on the target cells, allowing T cell-mediated killing.

FIG. 71—An embodiment of the Reverse CAR is illustrated. In this innovation, CAR T cells express the CH2CH3 region (with or without the expression of scFv). As illustrated by the data, the CH2CH3 region would allow for the recognition of fc-gamma receptor expressing cells such as macrophages resulting in the elimination of the fc-gamma receptor-expressing cells. Therefore, by expressing a molecule that can be recognized by the target cell, one can induce the killing of the target cell itself.

FIG. 72—This is a different example of the same embodiment previously described in FIG. 72. In this case, target cells recognize a molecule expressed by the T cells (CH2CH3 region) while containing only co-stimulatory endodomains such as CD28. Therefore, once the T cells get recognized by the macrophages, this will induce dimerization of the molecule and T cell proliferation, but not killing as the CD3zeta is not incorporated within the molecule.

FIG. 73—In this example of the Reverse CAR, T cells express a molecule that can be recognized by macrophages (CH2CH3) while the endodomains will contain the CD28 and CD3zeta. Therefore once T cells get recognized by macrophages, this will induce: (i) killing of macrophages by activation of CD3zeta and, (ii) T cell proliferation by activation of CD28.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

### I. General Embodiments

The present disclosure provides methods of optimizing engineered chimeric receptors for use in immune cells for immunotherapy such that the cells are efficacious and also able to proliferate sufficiently in vivo. The immune cells may be of any kind, but in at least some cases they are T cells, NK cells, NK T cells, B cells, monocytes, macrophages, dendritic cells, and so forth. In particular embodiments the receptor comprises at least two components separated by a spacer, and the length and/or content of the spacer in the receptor may be optimized to render the cells effective for therapy without significantly negatively impacting the ability of the cells to proliferate and expand in vivo. Such optimization balances the negative effects of tonic signaling that accelerates cell growth and cell aging (for example) with the positive aspects of effective targeting of a particular antigen to which the receptor is targeted and subsequent lysis of a cell expressing the antigen.

In certain embodiments, the receptor targets a tumor antigen. In methods of the disclosure, an antigen to which the receptor is desired to be targeted is known. In such cases, a spacer is optimized that separates an antigen recognition domain that binds the antigen from another component of the receptor. In at least specific cases, a spacer of the receptor is optimized by intentionally manipulating its length and/or content to permit cells that express the receptor to have a suitable balance between efficacious targeting of the antigen yet sufficient in vivo cell expansion. The manipulation(s) of the spacer can result in enhanced T-cell migration in addition to optimal antigen recognition and in vivo persistence.

### II. Tonic Signaling

In particular embodiments of the disclosure, chimeric receptors may be designed and/or tested for the extent to which cells that express them will elicit tonic signaling, which is the spontaneous dimerization/multimerization of transgenic molecules in the absence of an antigen.

One or more components of the receptor may be specifically designed, manipulated, and/or modified such that cells that express the receptor are not subject to accelerated cell growth and aging, thereby permitting the cells to have enhanced in vivo longevity. In particular cases, a spacer within the receptor is configured to permit the cells that express the receptor to avoid tonic signaling or at least to elicit a reduced level of tonic signaling than if the spacer had not been so designed, manipulated, and/or modified.

Although tonic signaling can be measured for one or more particular receptor configurations by any one or more methods, the tonic signaling may be a direct measurement or an indirect measurement of cell viability, including cell aging and/or growth.

In some cases, tonic signaling is measured based on the state of metabolic activity, for example as a measure that reflects that the T cells are more activated. Although their metabolic activity may be measured in any one or more ways, in specific embodiments the level of one or more compounds produced by the cells is measured, for example excreted into the supernatant of the cells in culture. Although the compound may comprise glucose and/or lactate, in some embodiments, the compound is another metabolite. In some cases, the ratio of one compound to another is a measure of tonic signaling, including the ratio of glucose to lactate, for example. In some cases, a glucose and lactate ratio can be used to identify the potential for tonic signaling (this may occur early in the culture of the cells, for example between 2-3 days after transduction).

In some embodiments, one can measure any indicator of T cell activation as a gauge of tonic signaling. In specific embodiments, an indicator of T cell activation associated with early stages of tonic signaling includes measurement of the levels of CD25, CD69, CD27, CD28, CD95, CD43, KLRG1, CD4OL, CD137, CD137L, or CD134 in the cells. This was also positively correlated to cell size. During intermediate stages of tonic signaling, one can determine the production of INFγ IL2, TNFα, INFb, GMCSF, perforin, IL13, IL4, TGFb without stimulation. During later stages of tonic signaling, one can identify phenotypical changes related to T cell aging, such as memory phenotype based on the expression of CCR7 and CD45RO/CD45RA, and CD27/CD28.

In certain cases, tonic signaling is measured as it relates to T cell activation by assaying for increased cytokine production from the cells, including without stimulation, for example. Any cytokine may be measured, but in specific embodiments the cytokine is interferon gamma, IL2, TNF, INFb, GMCSF, perforin, IL13, IL4, TGFb, or a combination thereof.

The presence of a chronic activation may be determined, for example by measuring whether or not there is a sustained high level of one or more particular markers, such as CD25, CD69, CD27, CD28, CD95, CD43, KLRG1, CD4OL, CD137, CD137L, and/or CD134. In specific embodiments, one can measure phosphorylation of the CD3 ζchain (phospho-CD3) in the absence of cognate antigen stimulation as evidence for tonic signaling. Evidence of tonic signaling may also be reflected in the state of cell cycle progression in the absence of antigenic stimulation, for example by determining whether or not there is a greater transition from a resting stage (G₀) to G₁, S, and G₂/M phases.

One may utilize phenotypic analyses to examine memory phenotypes as a measure of the cell aging process. For example, one may assay for CCR7 and/or CD45RO and determine the levels of naïve T cell populations over time and/or the levels of effector memory T cells over time. Such assays are indicative of the influence of the particular receptor molecule being tested on an acceleration (or not) of a cell aging process.

### III. Measurement of Efficacy of the Engineered Receptor(s)

In addition to measuring tonic signaling for cells that express the chimeric receptor, one can measure the efficacy of the receptor itself using one or more methods that are indicative of the function of the receptor (generally speaking, to target its antigen and/or elicit cell killing of cells that express the antigen). In specific embodiments, one or more of the following may be measured: 1) the efficacy of the binding of the antigen recognition domain to an antigen; 2) an in vitro killing assay of one or more cells expressing the receptor; 3) an in vivo assay measuring tumor size following delivery of cells expressing the receptor; 4) cytokine production of one or more cells expressing the receptor; 5) the in vivo proliferation of one or more cells that express the receptor; 6) the antitumor activity of the receptor; 7) cell phenotype, and/or 8) cell size.

The efficacy of binding of the receptor to its target antigen may be evaluated. Such binding may occur in a variety of ways, but in at least specific cases it occurs by exposing CAR expressing T cells to a serial dilution of antigen-expressing targets. Antigen recognition properties may be assessed by an in vitro killing assay. For example, one may utilize a standard chromium-51 (Cr⁵¹) release assay or may utilize co-culture experiments where cancer cells are co-cultured with receptor-bearing cells for a period of time, followed by FACS analysis, for example.

In particular cases, an in vivo model is employed to measure the in vivo anti-tumor potential of receptor-expressing cells by engrafting tumor cells onto mice and then treating the tumor with sufficient amounts of the cells.

In other cases, one or more particular assays do not include killing assays but may instead assay one or more other biological properties of the cells, such as cytokine production (for example, interferon gamma, IL2, TNF, INFb, GMCSF, perforin, IL13, IL4, and/or TGFb).

In specific embodiments, one can measure efficacy of the receptor by assaying for diminished Fc-FcγR interactions (for example, as measured in vitro by co-culturing macrophages and CAR T cells). In specific embodiments, one can evaluate the in vitro or in vivo T cell response when the CAR T cells have been exposed to FcγR-expressing cells such as macrophages.

One can also monitor the migration of the receptor-expressing cells to determine the ability of the engineered receptor-expressing T cells to egress from the lungs, for example using sequential luminescence imaging. Migration of the cells from the lungs to either tumor or secondary lymphoid tissue is favorable for the cells.

In specific embodiments, parameters that may be used to predict the efficacy of a CAR include the following: (i) T cell phenotype with a high content of naïve and central memory cells and in specific cases comprises cells with a high content of CCR7+ at 30% on Day 14 in absence of the antigen or a CCR7+ content that resembles the % observed in non-transduced T cells under the same culture conditions; (ii) another important characteristic that can predict T cell function is the growth pattern that resembles non-transduced T cell in the absence of the antigen; and/or (iii) the killing of target cells that express the antigen. In specific embodiments, a CAR is desirable if condition (i) and/or (ii) are present along with (iii).

### IV. Chimeric Fc receptor Target Molecules and Manipulations Thereof

In the present disclosure, a configuration of an engineered chimeric receptor is determined and/or the receptor is produced upon analysis of the efficacy of the receptor to bind its target (or efficacy of cells that express the receptor) balanced with the in vivo persistence of cells that express the receptor. Efficacy of cells that express the receptor includes at least the antitumor activity for the cells that express the receptor that is designed to target a tumor antigen. In particular embodiments, the spacer is of a determined length and/or content and the receptor is tested based upon one or more permutations of the length and/or content of the spacer.

In particular, the spacer length and/or content are specifically and deliberately selected for use in the engineered chimeric receptors, including to be tested using methods of the disclosure and ultimately, if shown to be suitable, to be utilized in therapeutic cellular immunotherapy with cells expressing the receptor. This is opposed to spacer length and/or content that is selected by chance or by routine, without employing methods of the disclosure to examiner the merit of the particular spacer.

In specific embodiments, the spacer separates two components on a single molecule and operably links the two components. In specific cases, the spacer in the receptor separates an antigen recognition domain that targets an antigen for the receptor, such as a tumor antigen, from an endodomain that activates the cell upon stimulation following binding of the antigen. In specific cases, the spacer could be at least a part of any extracellular amino acid sequence present in particularly Type 1 transmembrane proteins such as CD8, CD4, CD19, CD20, and/or CD28.

For a nucleic acid molecule that encodes the receptor, the configuration of the spacer in a 5′ to 3′ direction of a single nucleic acid molecule is such that the spacer is 3′ to one component on the molecule and 5′ to another component on the same molecule. For a single receptor polypeptide the configuration of the spacer in an N-terminal to C-terminal direction is such that the spacer is on the N-terminal side of one component on the molecule and on the C-terminal side of the other component on the molecule. Additional components for the receptor may be present other than the two components that immediately flank the spacer. For example, when the receptor is a chimeric antigen receptor, immediately downstream of the spacer there may be one or more costimulatory domains optionally followed by a CD3 zeta chain.

In particular embodiments, the spacer is modified to achieve a suitable equilibrium between the strength of the receptor function itself and the in vivo vigor of proliferation of cells that express the receptor. The condition of the in vivo proliferation may be determined in vivo or it may be extrapolated from in vitro cell proliferation studies.

In some cases, the length of the spacer is tested and/or manipulated for its influence on the balance between receptor efficacy and in vivo persistence of the cells that express the receptor. The length may be of any kind, but when the length is long (for example, >150 amino acids) or short (for example, <50 amino acids), as opposed to intermediate (for example, 50-150 amino acids), the cells are more prone to be able to recognize the antigen target. In particular embodiments, the following lengths of particular hinges and domains is as follows: IgG1 hinge: 12aa; IgG1 CH2: 113aa; IgG1 CH3: 107aa; IgG2 hinge: 12aa; IgG2 CH2: 109aa; IgG2 CH3: 107aa.

In certain cases, the content of the spacer is tested and/or manipulated for its influence on the balance between receptor efficacy and in vivo persistence of the cells that express the receptor.

In cases wherein the engineered receptor is a CAR, they CAR may target any antigen, including any tumor antigen. In specific cases, the tumor antigen is TEM1, TEM8, EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE Al, HLA-A2, IL11Ra, IL13Ra2, KDR, Lambda, Lewis-Y, MCSP, Mesothelin, Mucl, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracellular matrix of tumors, and so forth.

### V. Chimeric Fc Receptor Target Molecules and Uses Thereof

In some cases, the binding of a cell that expresses a receptor that comprises an IgG Fc domain (including the CH2CH3 domain, in at least some cases) to a Fc-gamma receptor-expressing cell is utilized to an advantage by targeting of the Fc-gamma receptor-expressing cells for their destruction. The elimination is beneficial to those individuals in which excessive levels of Fc-gamma receptor-expressing cells are detrimental, and such molecules may be referred to as chimeric Fc receptor target molecules, or reverse CARs. Thus, in specific embodiments, cells bearing chimeric Fc receptor target molecules are utilized to target Fc gamma receptor (FcγR)-bearing cells for the purpose of their destruction.

In particular embodiments, therapeutic amounts of cells bearing chimeric Fc receptor target molecules are provided to an individual in need thereof, such as an individual with any medical condition that has inflammation as a symptom. In specific cases, the medical condition is lung cancer, arthritis, multiple sclerosis, diabetic ulcers, atherosclerosis, asthma, sepsis, cardiovascular disease, or Alzheimer's Disease, for example.

In some embodiments, cells expressing one or more chimeric Fc receptor target molecules would recognize a Fc-gamma receptor-expressing cell (for example, a macrophage) because the CH2CH3 region would bind the Fc-gamma receptor, but the particular chimeric Fc receptor target molecule lacks a domain for cell activation (such as, lacks CD3zeta to be activated). In these cases, the Fc-gamma receptor-expressing cell is therefore not killed. In cases wherein the more chimeric Fc receptor target molecule lacks CD3 zeta but comprises one or more co-stimulatory molecules, the expansion of the cells that express the chimeric Fc receptor target molecule is enhanced. In particular cases, such cells are utilized in lung cancer, arthritis, multiple sclerosis, diabetic ulcers, atherosclerosis, asthma, sepsis, cardiovascular disease, or Alzheimer's Disease.

### IV. Pharmaceutical Compositions

Provided herein are pharmaceutical compositions comprising the genetically engineered immune cells that express engineered chimeric receptors, such as CARs.

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration into an individual, including for direct injection into a tumor. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition(s) of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. An example of a dosage for administration might be in the range of 1E+06 cell/m², 10E+06 cell/m², 100E+06 cell/m², 1000E+06 cell/m², and so forth. Progress can be monitored by periodic assessment.

The cell compositions of the disclosure may be administered locally or systemically. Administration may generally be parenteral, e.g., intravenous; the cellular composition(s) may also be administered directly to the target site, e.g., by biolistic delivery to an internal or external target site or by catheter to a site in an artery. In an embodiment, the pharmaceutical composition is administered subcutaneously and in another embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous receptor constructs or nucleic acid molecules or vectors encoding the same (as described in this disclosure), further biologically active agents, depending on the intended use of the pharmaceutical composition.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR molecule as described herein and/or regulatory elements therefor.

In particular embodiments, there are one or more apparatuses in the kit suitable for extracting one or more samples from an individual. The apparatus may be a syringe, scalpel, and so forth.

In some cases, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### VI. Therapeutic Uses of Engineered Chimeric Receptors and Host T-cells Comprising Same

In various embodiments engineered chimeric receptor constructs, nucleic acid sequences, vectors, host cells , as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer having solid tumors, for example.

In particular embodiments, provided herein is a method of treating an individual for cancer, comprising the step of providing a therapeutically effective amount of a plurality of any of cells of the disclosure to the individual. In certain aspects, the cancer is a solid tumor, and the tumor may be of any size. In certain aspects, the method further comprises the step of providing a therapeutically effective amount of an additional cancer therapy to the individual.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, cells, receptor constructs, nucleic acid molecules and vectors that can administered either alone or in any combination using standard vectors and/or gene delivery systems, and in at least some aspects, together with a pharmaceutically acceptable carrier or excipient. In certain embodiments, subsequent to administration, said nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject or individual in the need thereof an effective amount of immune cells, e.g., T cells or cytotoxic T lymphocytes, harboring an engineered chimeric receptor (such as a CAR); a nucleic acid sequence encoding same; a vector comprising a nucleotide sequence encoding same and/or produced by a process as described herein.

Possible indications for administration of the composition(s) of the exemplary cells are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genitourinary tract, e.g. ovarian cancer, endometrial cancer, cervical cancer and kidney cancer, lung cancer, gastric cancer, cancer of the small intestine, liver cancer, pancreatic cancer, gall bladder cancer, cancers of the bile duct, esophagus cancer, cancer of the salivary glands and cancer of the thyroid gland. The administration of the composition(s) of the disclosure is useful for all stages and types of cancer, including for minimal residual disease, early cancer, advanced cancer, and/or metastatic cancer and/or refractory cancer, for example, wherein the cancer is associated with pathogenic vascularization.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Particular doses for therapy may be determined using routine methods in the art. However, in specific embodiments, the T cells are delivered to an individual in need thereof once, although in some cases it is multiple times, including 2, 3, 4, 5, 6, or more times. When multiple doses are given, the span of time between doses may be of any suitable time, but in specific embodiments, it is weeks or months between the doses. The time between doses may vary in a single regimen. In particular embodiments, the time between doses is 2, 3, 4, 5, 6, 7, 8, 9, 10, or more weeks. In specific cases, it is between 4-8 or 6-8 weeks, for example

Embodiments relate to a kit comprising an engineered receptor construct as defined herein, a nucleic acid sequence as defined herein, a vector as defined herein and/or cells expressing the receptor as defined herein. It is also contemplated that the kit of this disclosure comprises a pharmaceutical composition as described herein, either alone or in combination with further medicaments to be administered to an individual in need of medical treatment or intervention.

In particular embodiments, there are pharmaceutical compositions that comprise cells that express an engineered chimeric receptor. An effective amount of the cells are given to an individual in need thereof.

By way of illustration, cancer patients or patients susceptible to cancer or suspected of having cancer may be treated as follows. Cells, including T cells, modified as described herein may be administered to the patient and retained for extended periods of time. The individual may receive one or more administrations of the cells. In some embodiments, the genetically engineered cells are encapsulated to inhibit immune recognition and placed at the site of a tumor.

In particular cases the individual is provided with therapeutic T-cells engineered to comprise a CAR in which the spacer was designed and/or manipulated to avoid tonic signaling for cells that express the CAR. The cells may be delivered in the same or separate formulations. Upon multiple administrations, the cells may be provided to the individual in separate delivery routes. The cells may be delivered by injection at a tumor site or intravenously or orally, for example. Routine delivery routes for such compositions are known in the art.

Expression vectors that encode the engineered chimeric receptor can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either OMEGA or O-vectors. See, for example, Thomas and Capecchi, Cell (1987) 51, 503-512; Mansour, et al., Nature (1988) 336, 348-352; and Joyner, et al., Nature (1989) 338, 153-156.

The construct may be introduced as a single DNA molecule encoding at least the engineered chimeric receptor and optionally another gene, or different DNA molecules having one or more genes. In such cases the constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

The exemplary cells that have been engineered to include the engineered chimeric receptors are then grown in culture under selective conditions and cells that are selected as having the construct may then be expanded and further analyzed, using, for example; the polymerase chain reaction for determining the presence of the construct in the host cells. Once the engineered host cells have been identified, they may then be used as planned, e.g. expanded in culture or introduced into a host organism.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that each patient may be monitored for the proper dosage for the individual, and such practices of monitoring a patient are routine in the art.

In another aspect, provided herein is a method of treating an individual having a tumor cell, comprising administering to the individual a therapeutically effective amount of cells expressing at least the engineered chimeric receptor. In a specific embodiment, said administering results in a measurable decrease in the growth of the tumor in the individual. In another specific embodiment, said administering results in a measurable decrease in the size of the tumor in the individual. In various embodiments, the size or growth rate of a tumor may be determinable by, e.g., direct imaging (e.g., CT scan, MRI, PET scan or the like), fluorescent imaging, tissue biopsy, and/or evaluation of relevant physiological markers (e.g., PSA levels for prostate cancer; HCG levels for choriocarcinoma, and the like). In specific embodiments of the invention, the individual has a high level of an antigen that is correlated to poor prognosis. In some embodiments, the individual is provided with an additional cancer therapy, such as surgery, radiation, chemotherapy, hormone therapy, immunotherapy, or a combination thereof.

Embodiments relate to a kit comprising cells as defined herein, CAR constructs as defined herein, a nucleic acid sequence as defined herein, and/or a vector as defined herein. It is also contemplated that the kit of this disclosure comprises a pharmaceutical composition as described herein above, either alone or in combination with further medicaments to be administered to an individual in need of medical treatment or intervention.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### CAR-PSCA T Cells Exhibit Anti-Tumor Activity in Vitro but Fail to Exert in Vivo Anti-Tumor Effects in a Subcutaneous Xenograft Tumor Model

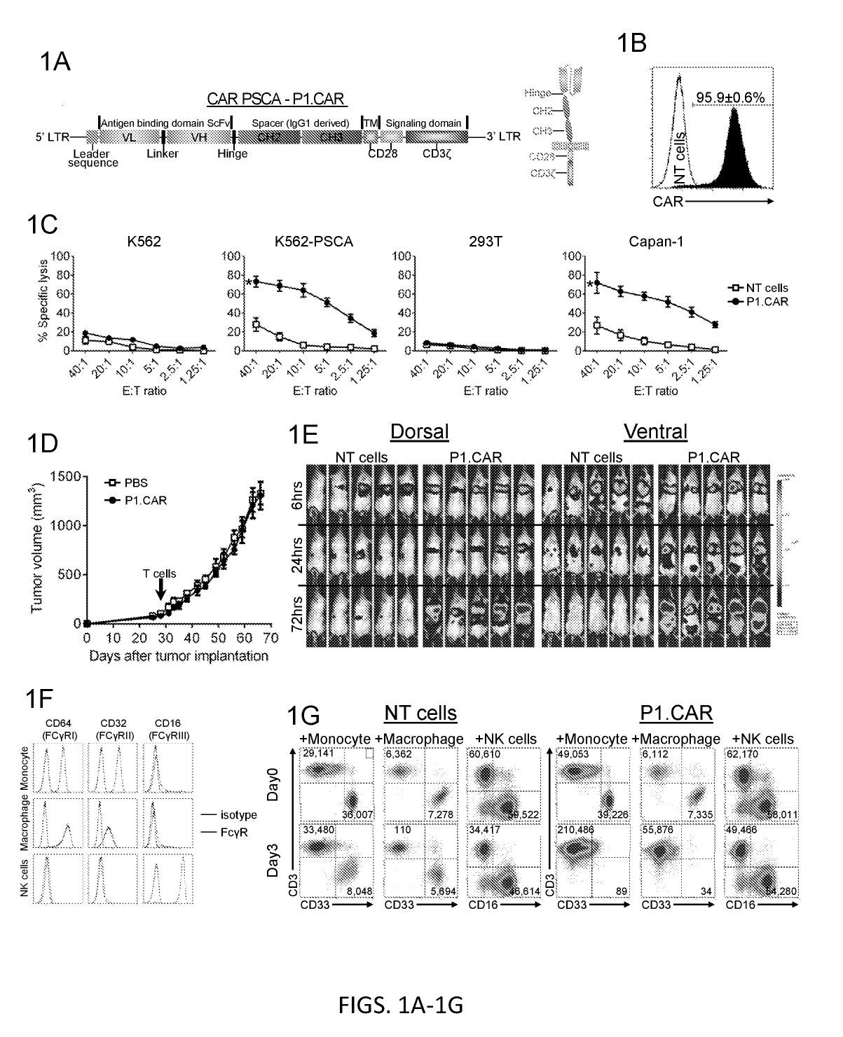

To target the tumor-associated antigen (TAA) PSCA, which is overexpressed in various solid tumors including prostate, pancreas and colon, the inventors constructed a retroviral vector encoding a humanized, codon-optimized, second generation CAR with an IgG1-derived hinge-CH2CH3, a CD28 transmembrane and signaling domain and the CD3t chain, which was considered a prototype CAR [P1.CAR] (FIG. 1A). This transgenic molecule was efficiently and stably expressed on the surface of activated T cells (95.9±0.6%, mean±S.E., n=8—FIG. 1B), conferring cells with the ability to specifically kill PSCA-expressing target cells (K562-PSCA; 73.1±5.9% and Capan-1; 72.0±11.1% specific lysis, mean±S.E., n=5, 40:1 E:T ratio) but not PSCA-negative targets, such as the K562 and 293T cells (19.0±2.6% and 8.4±2.0%, respectively), while non-transduced (NT) T cells produced only background levels of lysis (K562-PSCA; 27.9 ±7.0%, Capan-1; 26.9±8.9%, K562; 11.1±4.1% and 293T cells; 6.5±2.1% specific lysis, mean±S.E., n=5, 40:1 E:T ratio) (FIG. 1C). To evaluate the in vivo anti-tumor potential of these CAR T cells, 6-week old NSG (NOD.Cg-Prkdcscid I12rgtm1Wj1/SzJ) mice were engrafted with 5×10⁶ Capan-1 tumor cells subcutaneously (right flank) and after 28 days; when the tumor had reached a volume of >80 mm³, mice were treated with 10×10⁶ P1.CAR T cells labeled with GFP/firefly luciferase (FFluc). Surprisingly, when animals were treated with CAR T cells, the tumor volume continued to increase at a rate similar to that observed in control mice treated with PBS (FIG. 1D).

To assess whether deficient CAR T cell trafficking was responsible for this phenomenon, T cell migration was evaluated by performing sequential luminescence imaging in animals receiving either NT or P1.CAR T cells. As shown in FIG. 1E, NT T cells rapidly (within 24 hours) localized to secondary lymphoid tissues such as the spleen and lymph nodes. In contrast, P1 CAR T cells remained in the lungs, where the signal progressively increased over time. P1.CAR T cells failed to migrate to either the tumor or secondary lymphoid tissue. To investigate the mechanism behind this “non-specific” P1.CAR T cell expansion in the lungs, the inventors examined whether interactions between the CH2CH3 Fc region of the P1.CAR with Fcγ receptor-expressing cells could be responsible for this phenomenon (Hudecek, e al., 2015). Thus, NT and P1.CAR T cells were cultured at a 1:1 ratio with monocytes, macrophages and NK cells, all of which express different types of Fcγ receptor (CD64, CD32 and CD16) at varying intensities (FIG. 1F). As shown in FIG. 1G co-culture with monocytes and macrophages, which express CD64 and CD32, produced selective P1.CAR T cell expansion and resulted in the elimination of the Fcγ receptor-expressing cells. This phenomenon was not observed in the NK cell co-culture, suggesting that this recognition was mediated through interaction with the Fcy receptors I and II and not through CD16 (FIG. 1G).

### Example 2

### Modification of the CH2CH3 Spacer Improves Tumor Localization

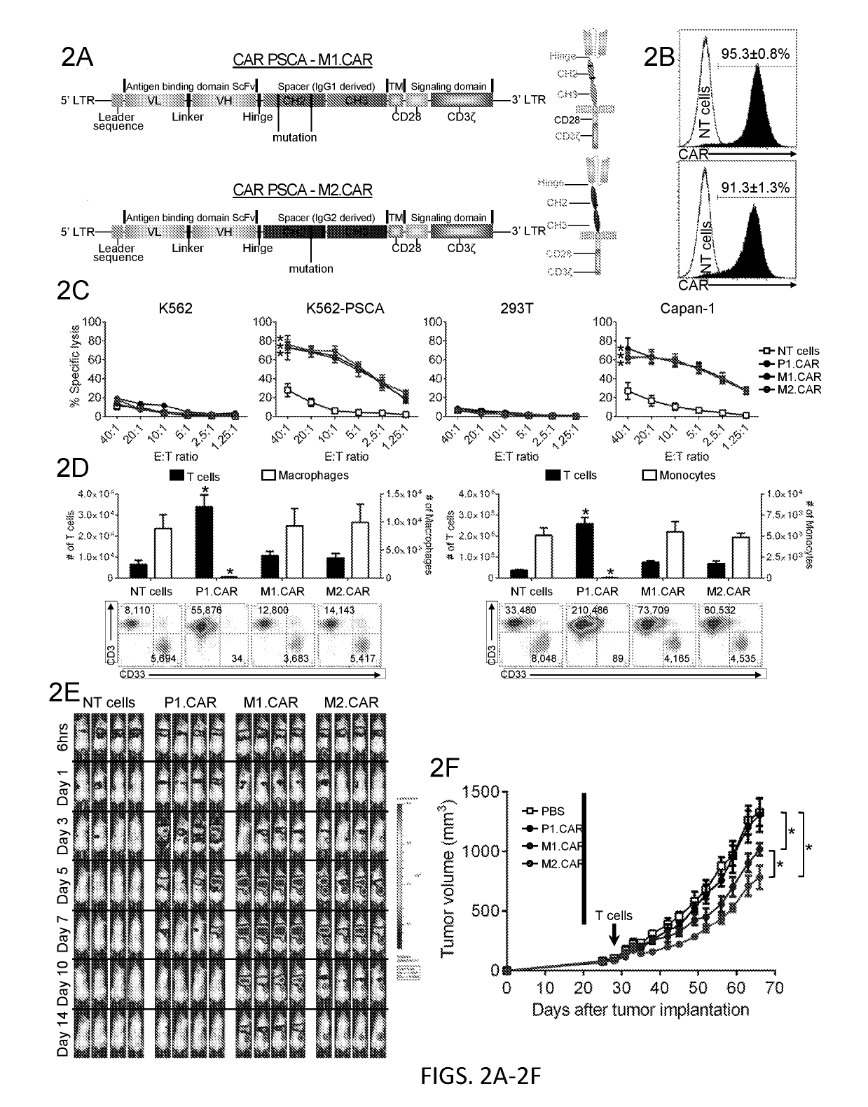

To abrogate Fcy receptor recognition, the FcγR-binding domains of the CH2CH3 region of the P1.CAR were modified as follows; (i) amino acids ELLG (position 233-236) and N (position 297) in the IgG1 CH2 region were mutated to PVA and Q, respectively [M1.CAR] (Hudecek, et al., 2015) and (ii) the CH2CH3 IgG1 framework was substituted for that of IgG2, reported to have the lowest Fcγ receptor recognition (Bruhns, et al., 2009; Jonsson, et al., 2012; Overdijk, et al., 2012), and amino acid N (297) was mutated to Q [M2.CAR] (FIG. 2A). Subsequently, it was investigated whether these modifications were sufficient to restore the migratory capacity of the CAR T cells. As shown in FIG. 2B, both the M1 and M2.CARs could be expressed at high levels on CD3/28-activated T cells (95.3±0.8% and 91.3±1.3%, respectively, mean±S.E., n=8), enabling cells to specifically kill PSCA⁺ targets (72.8%±12.9% and 61.5%±5.4% specific killing for M1.CAR and 75.8%±5.5% and 63.2±6.1% for M2.CAR against K562-PSCA and Capan-1, respectively, mean±S.E., n=5, 40:1 E:T ratio), with only background levels of killing against the control PSCA31 targets (K562 and 293T) (FIG. 2C). To investigate whether the modifications mitigated Fcγ receptor-mediated recognition, NT, P1, M1 and M2.CAR T cells were co-cultured with monocytes or macrophages (FIG. 2D) and after 3 days quantified residual cells by flow cytometry. As before, co-culture with P1.CAR T cells resulted in the elimination of macrophages/monocytes, while in the M1 or M2.CAR T cell co-cultures there was a profile similar to NT T cells with retention of macrophages/monocytes and limited T cell expansion, suggesting that the modifications had successfully minimized Fcγ receptor recognition (FIG. 2D). However, while M1 and M2.CARs had a similar reactivity against monocytes and macrophages in vitro, in vivo M2.CAR T cells were able to mobilize more efficiently from the lungs to the tumor site when compared to M1.CAR T cells (FIG. 2E), but, despite achieving tumor infiltration, the anti-tumor response to M2.CAR T cells was less than expected (FIG. 2F)—for reasons unrelated to target antigen expression, highlighting the need for further CAR optimization to improve long-term T cell persistence.

### Example 3

### Car T Cell Senescence

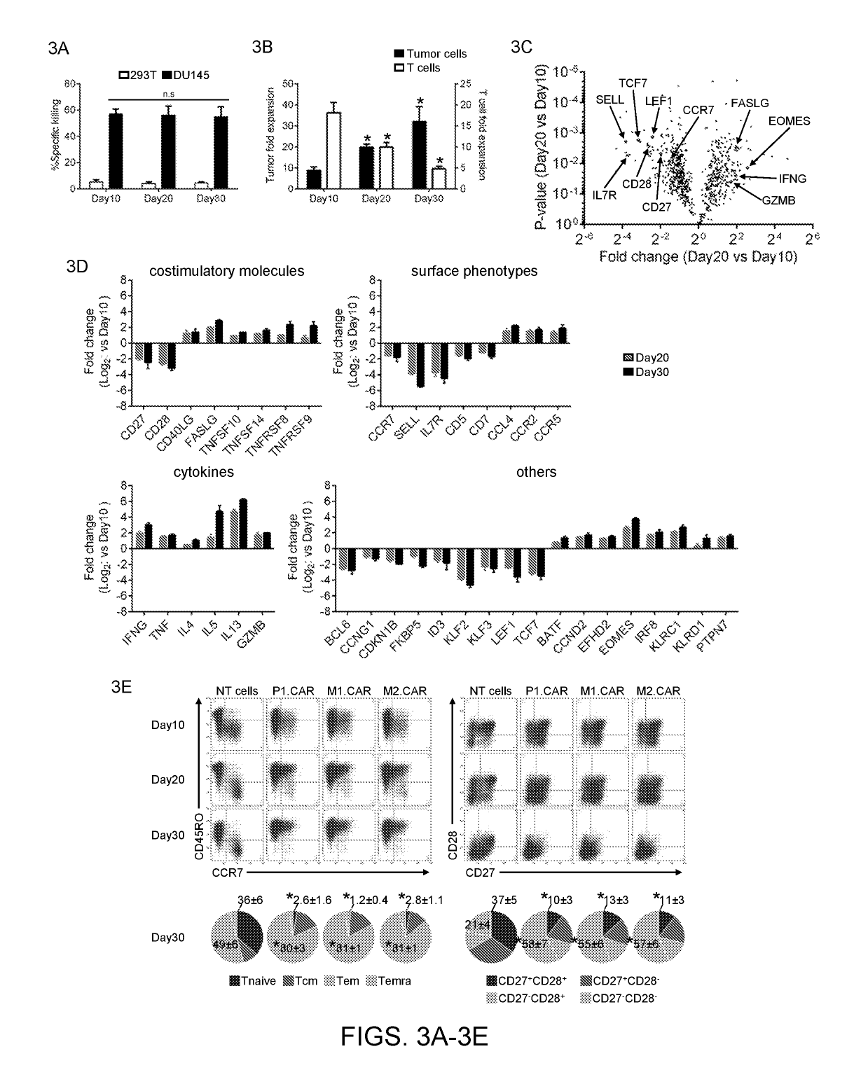

To determine if the lack of anti-tumor function of CAR T cells could be explained by a limited proliferative T cell capacity, a prospective in vitro study was conducted in which P1.CAR T cells were cultured in media supplemented with recombinant IL2 (without antigen/Fc receptor stimulation). Subsequently their gene expression profile were examined, as well as their phenotypic and functional characteristics on days 10, 20, and 30 of culture. As shown in FIG. 3A, prolonged in vitro expansion period (Day10, Day20 and Day30 after transduction) did not impact their short term (4 hr) in vitro cytolytic activity and there was similar killing of PSCA⁺ DU145 cells over time (57.0±3.8% Day 10, 56.0±7.0% Day 20, 54.7±7.9% Day 30, mean±S.E., n=3, 40:1 E:T ratio) with no recognition of control 293T cells (5.3±1.5% Day 10, 4.1±1.2% Day 20, 4.5±0.9% Day 30, mean±S.E., n=3, 40:1 E:T ratio) (FIG. 3A). However, when the inventors performed a longer term (6 day) co-culture with DU145 at 1:2 (effector:target) using T cells cultured for different periods of time (Day10, Day20 and Day30), there was an inverse correlation between anti-tumor activity and culture period (tumor cell fold expansion: 8.8±1.5 Day 10, 19.8±1.5 Day 20, 32.0±7.2 Day 30—mean±S.E., n=4)—a phenomenon due, at least in part, to decreased T cell proliferation (T cell fold expansion: 18.1±2.4 Day 10, 10.0±1.1 Day 20, 4.8±0.6 Day 30—mean±S.E., n=4) (FIG. 3B). When gene expression profiles were compared of P1.CAR T cells cultured for different time period, genes related to naïve/central memory T cells, such as CCR7, SELL, CD27, CD28 were progressively downregulated over time. In contrast, genes related to effector T cells, such as EOMES, FASLG and GZMB were progressively upregulated, indicating that the prolonged culture period differentiated CAR T cells from naïve/central memory to effector T cells with less proliferative capacity (FIGS. 3C and 3D).

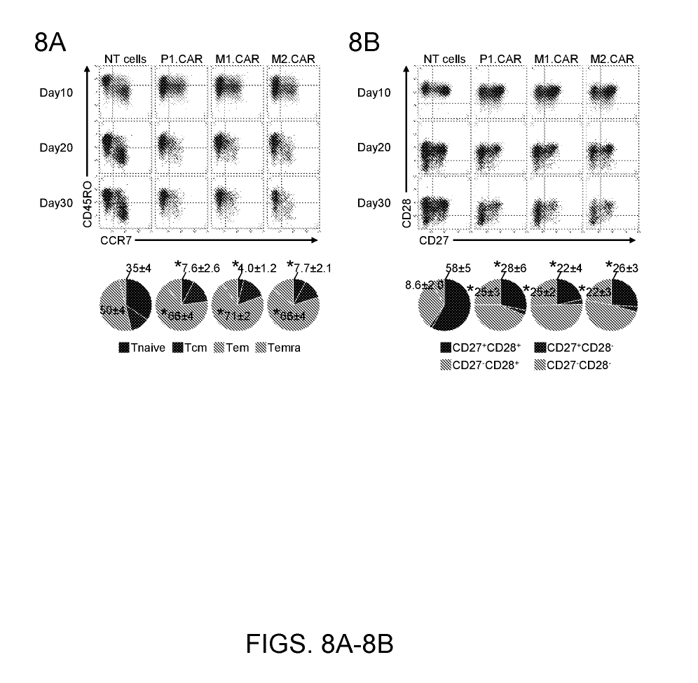

To confirm the microarray analyses and determine if the M1 and M2.CAR T cells exhibited a similar aging profile to that of the P1.CAR T cells, phenotypic analyses were next performed to examine memory phenotypes based on CCR7 and CD45R0 on both CD8⁺ (FIG. 3E—left) and CD4⁺ T cells (FIG. 8A). While NT cells retained naïve T cell population over time, there was a significant reduction of naive T cells in all three CAR-modified T cells (P1, M1 and M2) and a substantial increase in the frequency of effector memory T cells. Similarly, NT cells were able to retain both CD27 and CD28 expression over time, however, all three CAR T cells exhibited a progressive decline in expression of both molecules (FIG. 3E—right for CD8⁺ T cells and FIG. 8B for CD4⁺ T cells), thereby suggesting the influence of the CAR molecule on the accelerated T cell aging process.

### Example 4

### Tonic Signaling is Responsible for Accelerated CAR T Cell Aging

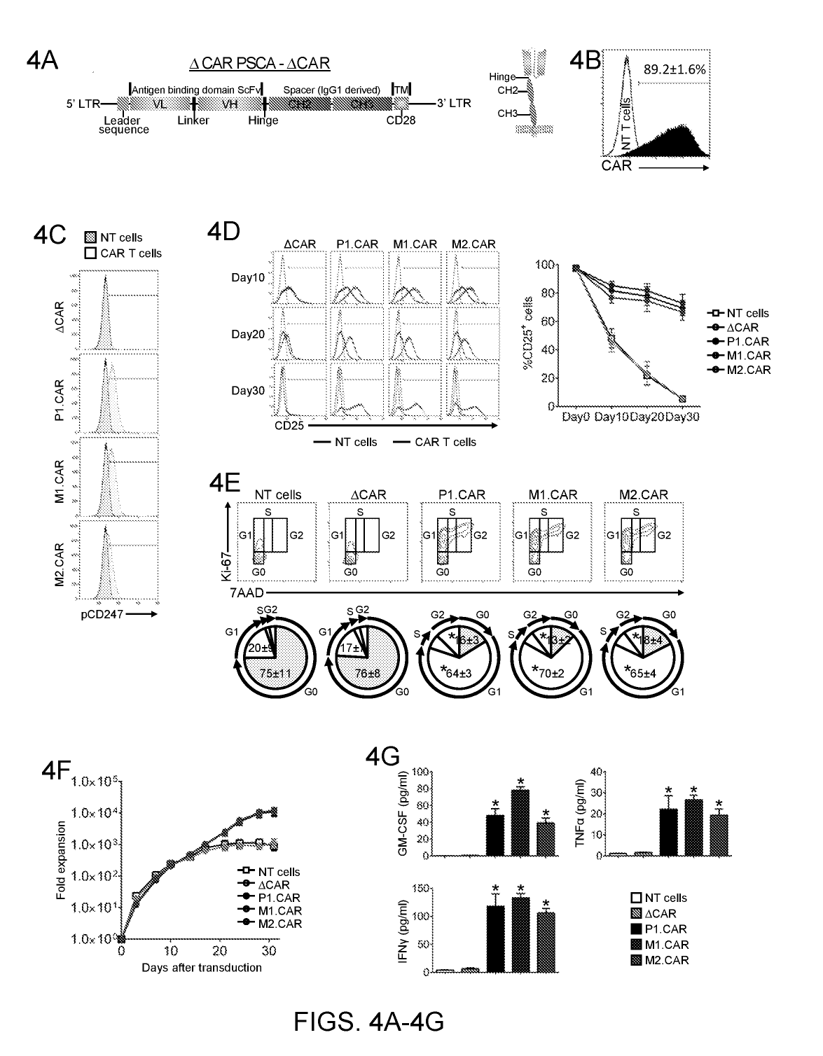

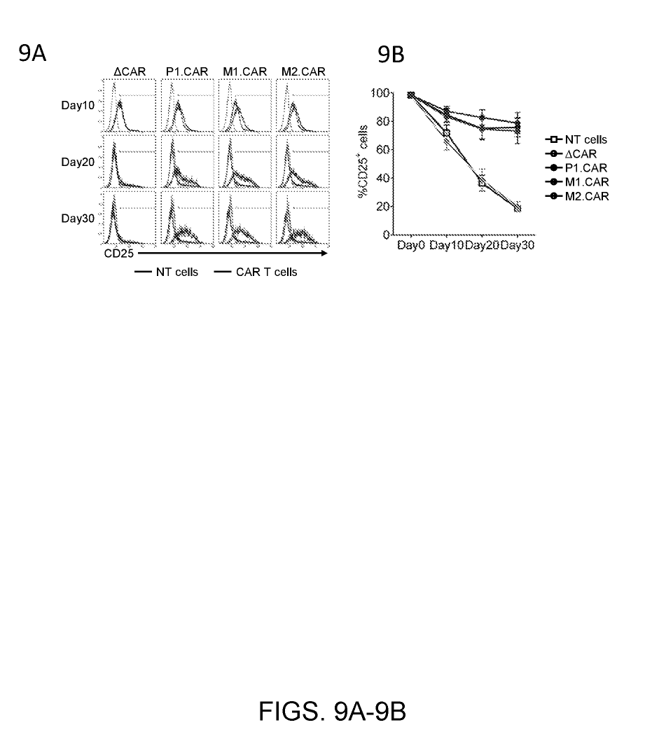

To further investigate whether these phenotypic and functional changes could be due to spontaneous CAR signaling, a truncated version of the P1.CAR lacking the intracellular signaling domains (ΔCAR—FIG. 4A) was generated, which was used to genetically modify T cells (89.2±1.6% transduction, mean±S.E. n=7) (FIG. 4B). Evidence of tonic signaling was sought by measuring phosphorylation of the C chain (phospho-CD3) in the absence of cognate antigen stimulation. As shown in FIG. 4C, all except the ΔCAR T cells exhibited evidence of CD3 signaling, resulting in a chronic activation state as measured by sustained high level CD25 expression in both CD8⁺ and CD4⁺ T cell subsets (FIG. 4D and FIG. 9). FIG. 4D (left panel) shows CD25 expression on CD8+T cells from a representative donor while the right panel summarizes CD25 expression on CD8+ T cells from the 6 donors screened (69.5%±5.3%, 72.8±6.2%, 66.6±5.8%, 5.4±0.9%, and 5.5±0.7% for P1, M1, M2, NT and ΔCAR T cells, respectively on Day 30, mean±S.E. n=6). As expected, this tonic signal promoted cell cycle progression as shown by greater transition from a resting stage (G₀) to G₁, S, and G₂/M phase for P1.CAR, M1.CAR and M2.CAR T cells (% of G₀ cells: 16.2±3.3%, 12.8±1.7%, 17.6±3.9%) than in NT and ΔCAR T cells (% of G₀ cells: 74.9±10.8% and 76.0±8.4%) (FIG. 4E—mean±S.E. n=3) and was consequently responsible for promoting exponential P1, M1 and M2 T cell growth in the absence of the antigenic stimulation (FIG. 4F) (1.1±0.3×10⁴, 1.1±0.3×10⁴, 1.2±0.3×10⁴, 0.9±0.3×10³ and 1.1±0.4×10³ fold expansion on Day31 for P1, Ml, M2, NT and ΔCAR T cells, mean±S.E., n=7) explained in part by a greater proliferative cell cycle. Finally, consistent with their activated profile, T cells modified with these constructs also exhibited increased cytokine production without stimulation (FIG. 4G) implicating tonic signaling as the underlying mechanism behind the accelerated cell growth and T cell aging.

### Example 5

### The CH2CH3 Spacer is Responsible for Tonic T Cell Signaling

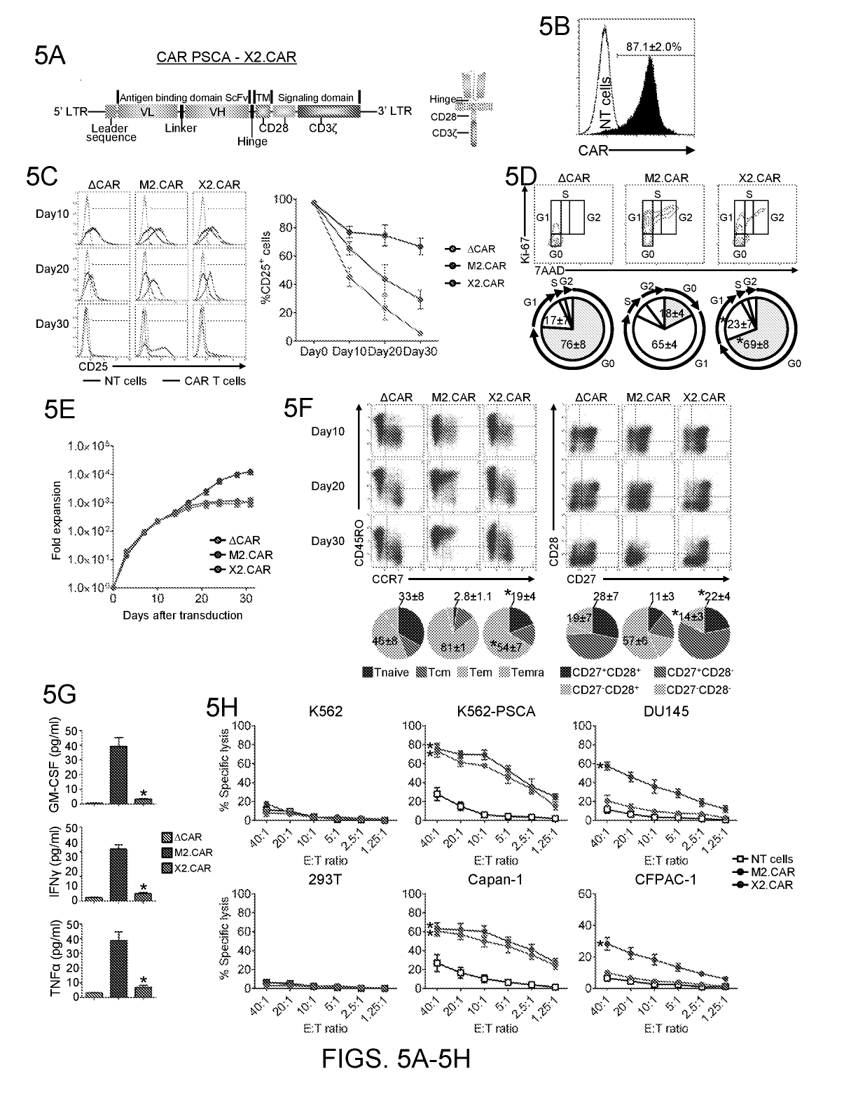

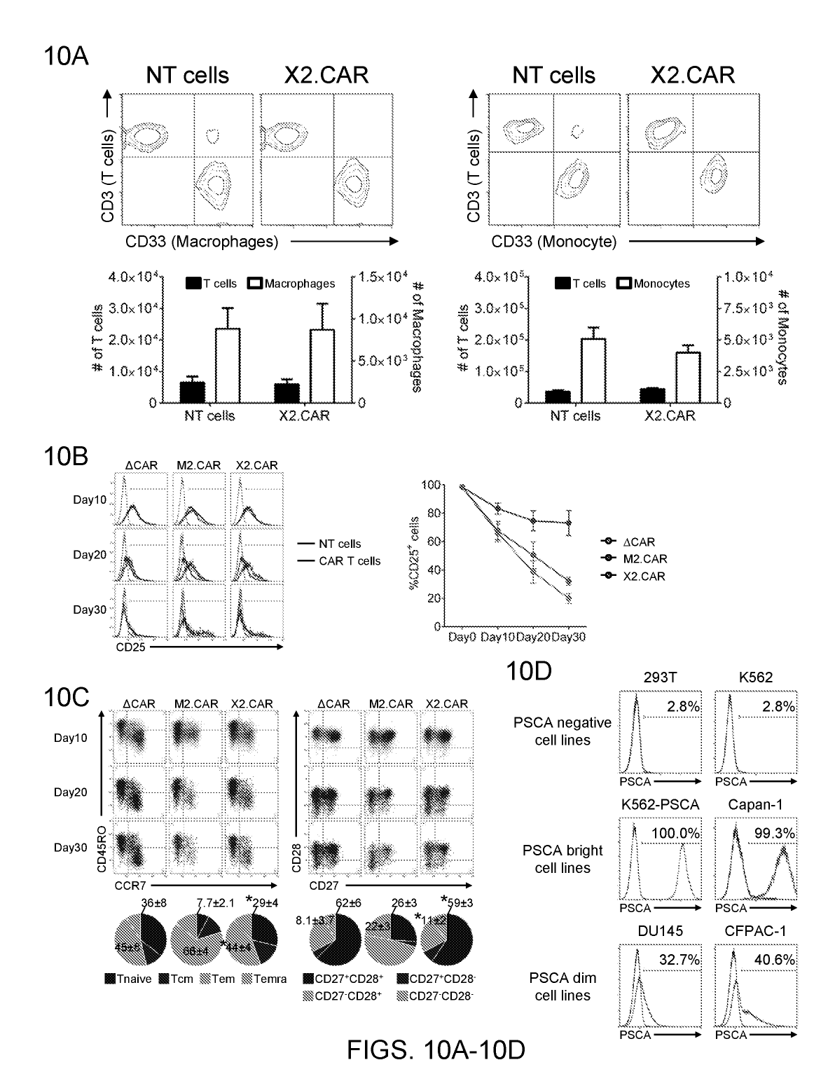

To determine if removing the CH2CH3 region entirely would abrogate tonic signaling, a new construct was generated with this region entirely deleted (X2.CAR, FIG. 5A), which could be detected on T cells using a human anti-FAB antibody (87.1±2.0%, mean±S.E., n=8) (FIG. 5B). As expected, in the absence of the CH2CH3 domain there was no elimination of monocytes or macrophages over 3 days (FIG. 10A). To evaluate the impact of CH2CH3 removal on tonic signaling the activation status of ΔCAR (negative control), M2.CAR (positive control) and X2.CAR T cells was monitored using CD25 cell surface expression as a readout. As shown in FIG. 5C and FIG. 10B, X2.CAR T cells exhibited a profile similar to that of ΔCAR T cells with basal CD25 expression over 30 days of culture. The left panel shows data from a representative donor while the right panel shows summary results from 6 donors tested. This non-activated state was corroborated by cell cycle analysis, demonstrating a maintenance of the Go by the X2.CAR T cells (69.2±8.4%, mean±S.E. n=3—FIG. 5D) resulting in decreased cell expansion (FIG. 5E), maintenance of an undifferentiated phenotype characterized by retention of naive-like T cells (19.2±4.0% vs 2.8±1.1%, X2 vs M2, Day 30, mean±S.E. n=6), effector memory T cells (53.7±6.9% vs 81.4±1.0%, X2 vs M2, Day 30, mean±S.E. n=6) and CD27/CD28 positive cells (21.9±4.0% vs 10.7±3.2%, X2 vs M2, Day 30, mean±S.E. n=6) (FIG. 5F and FIG. 10C) and diminished non-specific cytokine production (GM-CSF; 0.7±0.1, 39.2±3.4 and 3.4±0.4 pg/mL, TNFα; 1.6±0.1, 19.3±3.0 and 3.5±0.7 pg/mL and IFNγ; 6.8±0.8, 106.2±8.2 and 14.9±2.3 pg/mL for ΔCAR, M2 and X2 CAR T cells, respectively, mean±S.D.) (FIG. 5G). However, when the cytolytic capacity of X2.CAR T cells was assessed in a 4 hr ⁵¹Cr release assay, although the transgenic cells were effectively able to kill targets expressing high levels of PSCA (K562-PSCA and Capan-1—FIG. 10D), they demonstrated little/no recognition of tumor cells expressing low levels of PSCA, significantly impacting their ability to kill (20.7±5.8% vs 57.5±4.3%; X2 vs M2 for DU145, 9.9±1.5% vs 28.3±4.0%; X2 vs M2 for CFPAC-1) (FIG. 5H). These results suggest that although tonic signaling can be mitigated by removing the CH2CH3 region, this modification can adversely affect antigen recognition and subsequent target lysis.

### Example 6

### Incorporation of CH3 as a Spacer Decreases Cell Aging and Restores Cytoytic Abilities

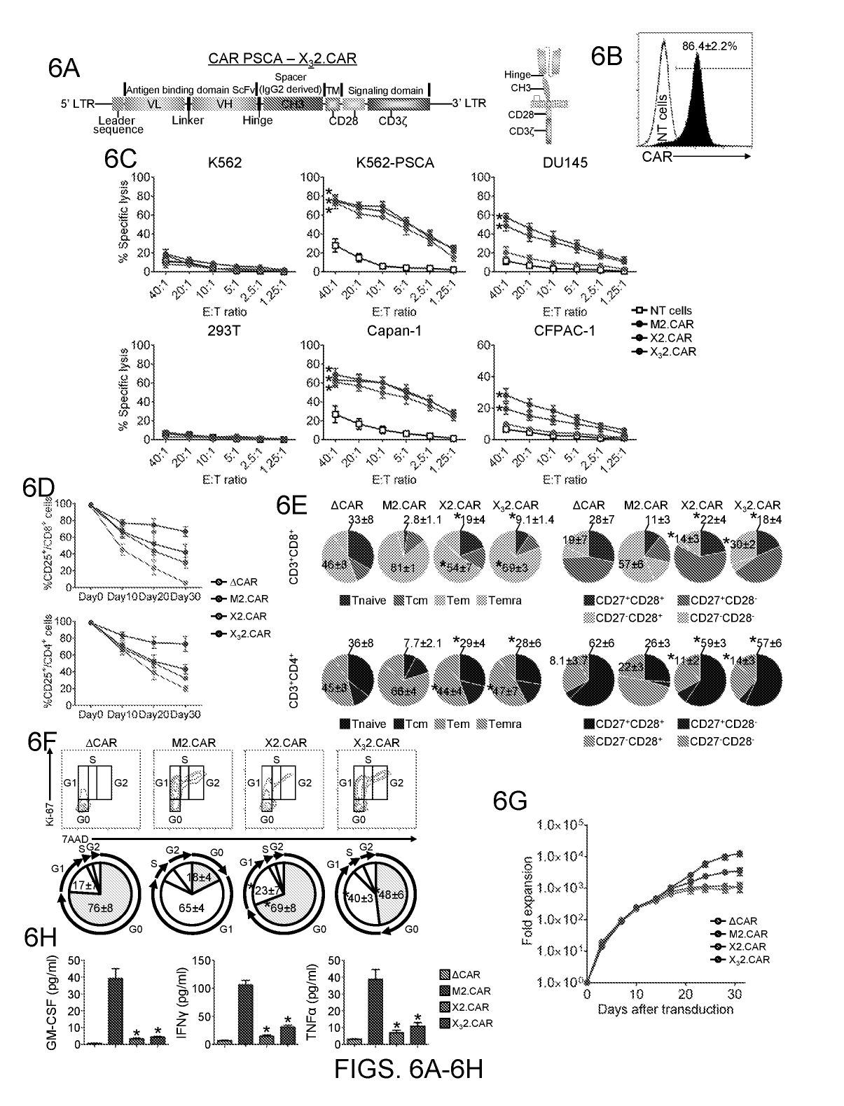

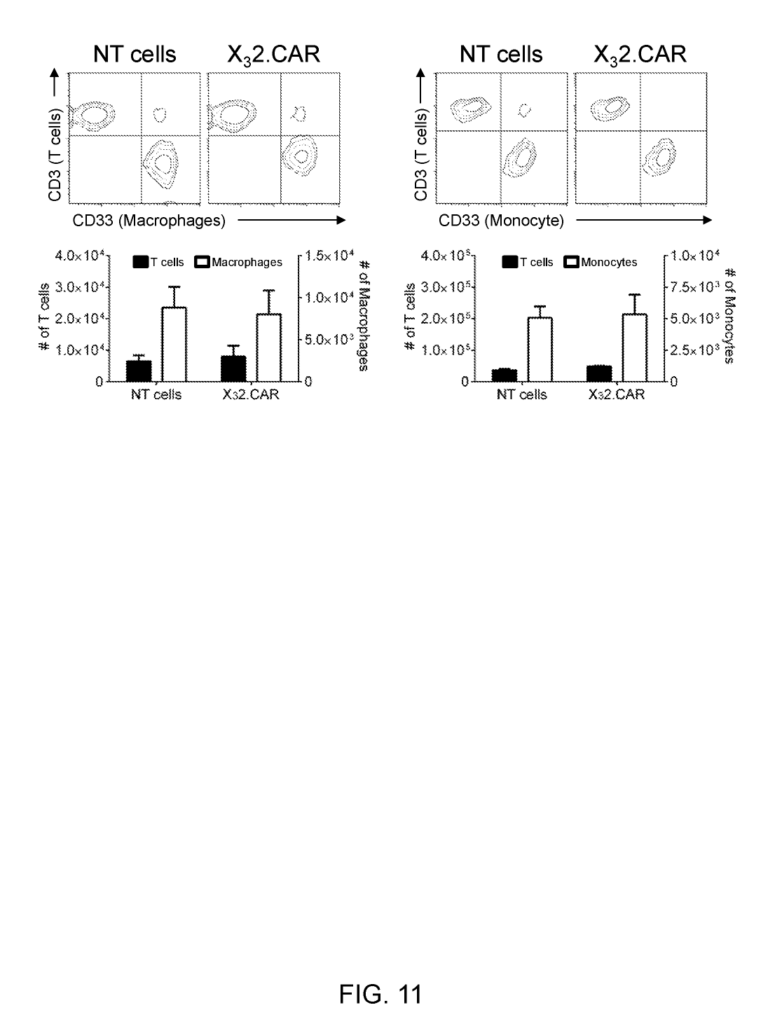

To generate an effective CAR that retained its cytolytic capacity, an additional vector was generated with an intermediate length spacer comprising only the IgG2 CH3 domain (X32.CAR—FIG. 6A), whose expression could be detected using the anti-FAB antibody (86.4±2.2%, mean±S.E., n=8, FIG. 6B). To first determine whether this construct enabled T cells to recognize and kill target cells expressing both high and low levels of target antigen, a chromium release assay was performed. As shown in FIG. 6C, inclusion of the CH3 region allowed transgenic T cells to recognize tumor cells expressing both high (K562-PSCA; 74.8±2.8%, Capan-1; 68.8±6.6%, mean±S.E., n=5, 40:1 E:T ratio) and low (DU145; 48.4±5.2%, CFPAC-1; 19.6±3.5%, mean±S.E., n=5, 40:1 E:T ratio) PSCA levels with no recognition of control K562 and 293T cells. Because this construct lacked the CH2 region, as expected there was no evidence of Fe-Feγ receptor interaction (FIG. 11). Furthermore, based on assessment of phenotype (FIGS. 6D, 6E), cell cycle status (FIG. 6F), cell expansion (FIG. 6G), and cytokine production (FIG. 6H) this enhanced anti-tumor activity was not gained at the expense of non- specific T cell activation since CD25 expression remained low over time and X32.CAR T cells retained a naive T cell population (CD8⁺; 9.1±1.4% vs 2.8±1.1%, CD4⁺; 27.6±5.8% vs 7.7±2.1%, X32 vs M2, Day 30, mean±S.E., n=6) and CD27/CD28⁺ (CD8⁺; 18.4±4.3% vs 10.7±3.2%, CD4⁺; 56.8±6.4% vs 26.1±2.7%, X32 vs M2, Day 30, mean±S.E., n=6). Consequently, cell expansion was decreased relative to M2.CAR T cells (3.4±0.9×10³ vs 12.7±2.7×10³ fold expansion on Day31, X32 vs M2 CAR T cells, mean±S.E., n=7—FIG. 6G) with minimal background cytokine production (GM-CSF; 4.3±0.3 vs 39.2±3.4 pg/mL, TNFα; 5.4±0.6 vs 19.3±1.7 pg/mL, IFNγ; 31.1±1.9 vs 106.2±4.7 pg/mL, X32 vs M2.CAR T cells, mean±S.D.—FIG. 6H).

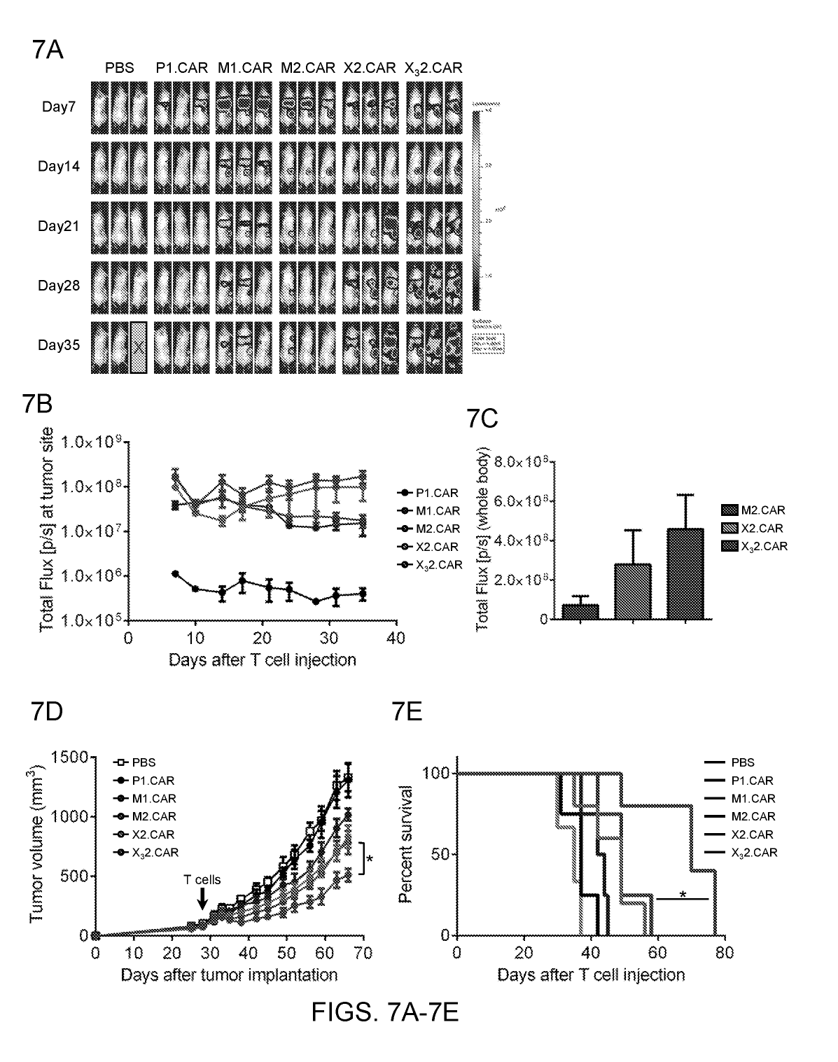

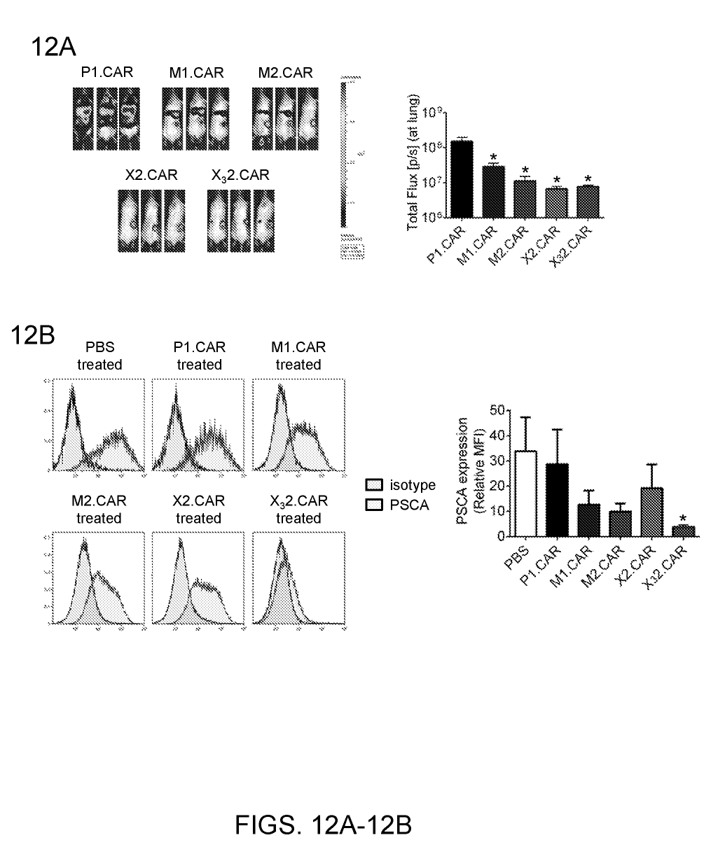

In vivo CAR anti-tumor activity - Based on previous in vitro data (presented in FIGS. 1, 2, 5, and 6), it was predicted that in vivo, (i) P1. CAR T cells would be trapped in the lungs and eliminated rapidly, (ii) M1 and M2.CAR T cells would traffic to the tumor (and secondary lymph nodes) but would not control tumors due to cell senescence as a consequence of tonic signaling, (iii) X2.CAR T cells would effectively traffic to the tumor, persist in vivo and produce immediate tumor killing but fail to eliminate residual tumor cells expressing low levels of target antigen, while (iv) X32.CAR T cells would effectively traffic to the tumor, persist in vivo and control tumor growth, resulting in a survival benefit. To assess if this was indeed the case, NSG mice were engrafted s.c. with Capan-1 cells and when the tumor had reached a volume of 80 mm³, animals were administered i.v. with 10×10⁶ CART cells (P1, M1, M2, X2 or X32). In vivo T cell migration and proliferation was monitored by luminescence imaging while tumor volume was measured by calipers. As shown in FIG. 7A, only M1, M2, X2 and X32 CAR T cells were able to escape the lungs (FIG. 12A), a phenomenon that correlated with a better in vivo T cell persistence at the tumor (FIG. 7B) and secondary lymphoid organs (FIG. 7C). Furthermore, while all 4 modified constructs delayed tumor growth in vivo to some extent, X32.CAR T cells exhibited the most efficient control of tumor growth by day 66 (tumor volume; 1330±115, 1309±143, 1018±51, 785±99, 833±94 and 511±53 mm³ for PBS, P1, M1, M2, X2 and X32.CAR T cell treated group, n=3-5/group, p<0.01 between M2 vs X32.CAR). Although tumor eventually relapse, this phenomenon was due to the growth of an antigen negative population (FIG. 12B), the incorporation of this adaptive CAR design improved several biological features resulting in the overall survival benefit (median survival; day 35, day 37, day 43, day 49, day 49 and day 70 for PBS, P1, M1, M2, X2 and X32.CAR T cell treated group, p<0.01 between M2 vs X32.CAR—FIG. 7C).

### Significance of Certain Embodiments

The inventors considered whether certain substitutions (Hudecek, et al., 2015) could restore the activity of the CAR.PSCA T cells in vivo, and while this did indeed diminish Fc-FcγR interactions (as measured in vitro by co-culturing macrophages and CAR T cells) only a fraction of T cells were able to localize at the tumor while a significant number of T cells remained trapped at the lungs (FIG. 2E). This highlighted the need for additional modifications to further reduce this interaction. Therefore, the inventors substituted the IgG1 framework of the exemplary CAR for that of IgG2 with the objective of abrogating Fc-FcγR interaction. Indeed, as illustrated in FIG. 2E, this additional modification did indeed allow T cells to egress the lungs and improved tumor site localization.

Although T cell migration and tumor localization are necessary pre-requisites for anti-tumor responses, they are not sufficient. Indeed, at the tumor site CAR T cells must proliferate, and persist in a functional state to provide long-term tumor control. However, the findings in this disclosure additionally highlight the importance of selecting a CAR whose configuration does not contain tonic signaling as this can result in the development of an adverse T cell phenotype.

To produce tumor elimination, the CAR scFv must engage with antigen and it is in this role that the spacer has traditionally been considered to play a key role by providing access to the epitope. This association was initially described by Moritz and colleagues, who demonstrated a direct correlation between the spacer length and the capacity of CAR ErbB-2 to engage with antigen (Moritz, et al., 1995). However, these results were not reproduced by Hornbach and colleagues using a CD30-targeted CAR (Hornbach, et al., 2000), implying that target epitope location is also central to this process. Indeed, subsequent studies using CARs targeting a range of antigens (CEA, NCAM, 5T4, CD19 (Guest, et al., 2005), MUC1 (Wilkie, et al., 2008), CD22 (Haso, et al., 2013), ROR1 (Hudecek, et al., 2013) and CD171 (Kunkele, et al., 2015) have borne out this assertion. Therefore, the present results indicate that the epitope location of PSCA to be proximal to the cell membrane with the use of a long CAR spacer resulted in the greatest target recognition. This must be balanced with in vivo proliferative capabilities, however.

### Example 7

### Observation of Consequences of Tonic Signaling

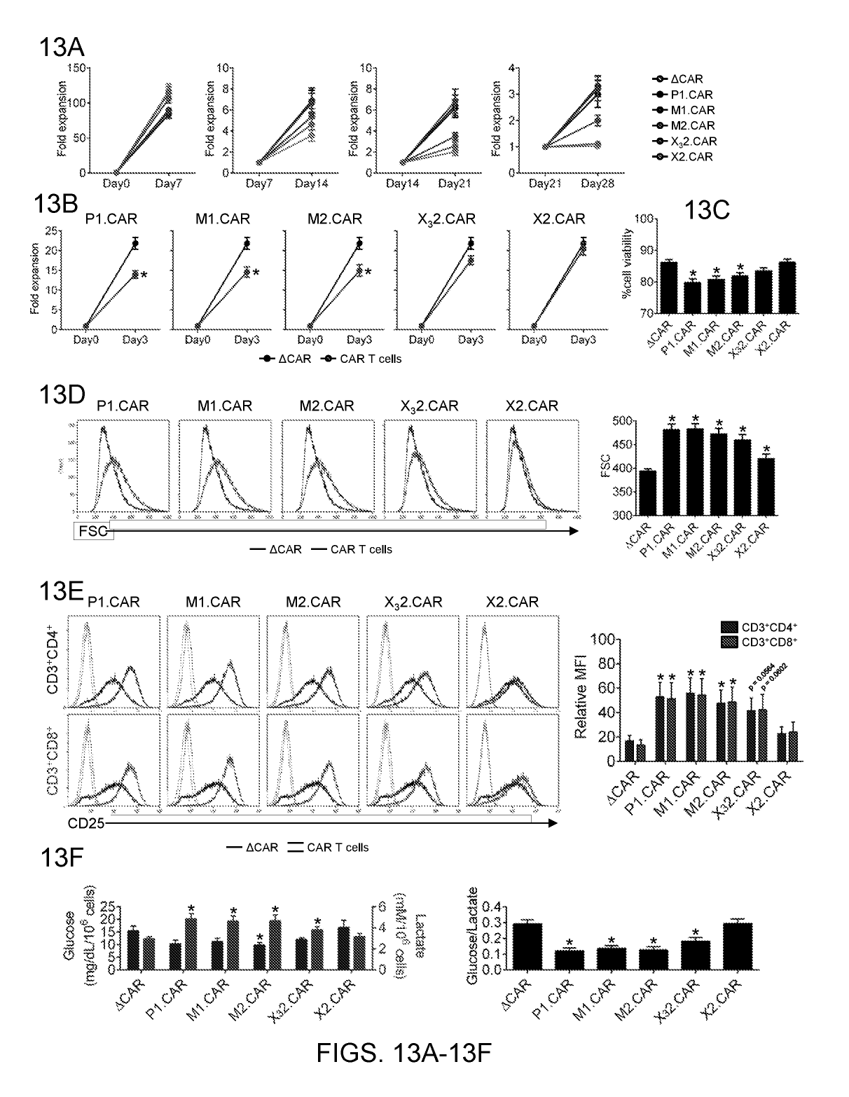

Although tonic signaling promotes cell expansion over time as measured by a longer time point, >10 days, (FIG. 6G and 13A), the inverse was observed in a shorter time point, <3 days, where cells expressing a CAR comprising a long CAR spacer did not expand compared to cells expressing a CAR comprising the short CAR spacer (FIG. 13B), in part, due to decreased cell viability (FIG. 13C). This phenomenon is a consequence of over activation caused by tonic signaling demonstrated by an increase in cell size (FIG. 13D) and upregulation of activation marker CD25 (FIG. 13E). Furthermore, the long CAR spacer drives a higher metabolic activity in cells expressing the CAR containing it as shown by a lower glucose/lactate ratio (FIG. 13F).

### Example 8

### Examples of Methods for Examples 1-7

Donors and Cell lines—Peripheral blood mononuclear cells (PBMCs) were obtained from healthy volunteers after informed consent on protocols approved by the Baylor College of Medicine Institutional Review Board. K562 (chronic erythloid leukemia cell line), 293T (human embryonic kidney cell line), Capan-1 (pancreatic cancer cell line), DU145 (prostate cancer cell line) and CFPAC-1 (pancreatic cancer cell line) were obtained from the American Type Culture Collection (Rockville, Md.). Cells were maintained in a humidified atmosphere containing 5% carbon dioxide (CO₂) at 37° C. K562 cells were maintained in RPMI-1640 media (GE Healthcare Life Sciences, Pittsburgh, Pa.) while 293T cells were maintained in Dulbecco's modified eagle medium (DMEM, GE Healthcare Life Sciences). Capan-1, DU145 and CFPAC-1 cells were maintained in Iscove's Modified Dulbecco's Medium (IMDM; Gibco BRL Life Technologies, Inc., Gaithersburg, Md.). Capan-1 cells were grown in IMDM containing 20% heat-inactivated fetal bovine serum (FBS) (Hyclone, Waltham, Mass.) with 2 mM L-GlutaMAX (Gibco BRL Life Technologies, Inc.) while other cell lines were grown in their specific media containing 10% FBS with 2 mM L-GlutaMAX.

Generation of retroviral constructs and retroviral transfection—A 2^{nd} generation CAR-PSCA was constructed that has IgG1-derived Hinge-CH2-CH3 as a spacer, followed by CD28 and CD3z intracellular domain (P1.CAR) using methods previously described (Anurathapan et al. Mol Ther. 2014 Mar; 22(3):623-33). To generate 2G.CAR-PSCA constructs with various spacers, the inventors synthesized DNA (Invitrogen, Grand Island, N.Y.) for the spacer region derived from IgG1-Hinge-CH2-CH3 with mutation (substitution of amino acid sequence from ELLG (position; 233-236 (EU numbering)) to PVA and N297Q (M1.CAR), derived from IgG2-Hinge-CH2-CH3 with mutation (N297Q) (M2.CAR), IgG2-Hinge (X2.CAR) and IgG2-Hinge-CH3 (X32.CAR). Spacer sequences in P1.CAR construct were replaced to new spacer by enzymatic digestion of the Bam1 and Pf1MI sites located before and after the spacer. The γ-retroviral vectors encoding the fusion protein (GFP/FFluc) were previously described (Vera, et al., 2009). Retroviral supernatant was produced as previously described (Leen, et al., 2014).

Generation of chimeric antigen receptor (CAR)-modified T cells—To generate CAR T cells, 1.0×10⁶ PBMCs were plated in each well of a non-tissue culture-treated 24-well plate that had been pre-coated with OKT3 (1 mg/ml) (Ortho Biotech, Inc., Bridgewater, N.J.) and CD28 (1 mg/ml) (Becton Dickinson & Co., Mountain View, Calif.). Cells were cultured in complete media (RPMI-1640 containing 45% Clicks medium (Irvine Scientific, Inc., Santa Ana, Calif.), 10% FBS and 2 mM L-GlutaMAX), which on day 1 after activation was supplemented with recombinant human interleukin-2 (IL2) (50 U/mL, NIH, Bethesda, Va.). On day 3, the retroviral supernatant was plated in a non-tissue culture-treated 24-well plate (1 mL/well) pre-coated with a recombinant fibronectin fragment (FN CH-296; Retronectin; TAKARA BIO INC, Otsu, Japan), and centrifuged at 2,000×g for 90 min. After removal of supernatant, OKT3/CD28-activated PBMCs (0.1×10⁶/mL) were resuspended in complete media supplemented with IL2 (100U/mL) and 2 ml was added to each well of a 24 well plate, which was subsequently spun at 400×g for 5 min, and then transferred to the 37° C., 5% CO₂ incubator. Subsequently, cells were split and fed every 2-3 days with fresh media plus IL2 (50 U/mL) (R&D Systems, Minneapolis, Minn.). To track T cell numbers overtime, viable cells were manually counted by trypan blue exclusion assay.

Generation of K562 modified to express PSCA—The tumor associated antigen PSCA was synthesized based on published sequences (Reiter, et al., 1998). The sequence was input into pVITRO1-blasti-mcs vector (Invivogen, San Diego, Calif.) by enzymatic digestion of Agel and Nhel site and transfected into K562 using GeneJuice® Transfection Reagent (EMD Millipore, Darmstadt, Germany). Transfected cells were selected and maintained in the presence of 10 ng/mL of Blasticidin (Invivogen).

Fcγ receptor-expressing cells preparation—Monocytes were isolated from PBMCs by using human CD14 microbeads (MACS system; Miltenyi Biotec Inc., San Diego, Calif.). Macrophages were generated by culturing monocytes with 100 ng/mL GM-CSF for 7 days. NK cells were expanded by stimulating 5×10⁶ PBMCs with 5×10⁶ irradiated K562-mbIL15-41BBL (Imai, et al., 2005; Fujisaki, et al., 2009) in the presence of 500 U/mL IL2 in G-Rex 10 device (Wilson Wolf Manufacturing, Minneapolis, Minn.) for 7 days as previously published (Crit Rev Oncog, et al., 2014), and then CD3 positive cells were depleted by using CD3 microbeads (MACS system; Miltenyi Biotec Inc.).

Flow cytometry—Cell surface stainin—The following antibodies were used in this study; CD3-PerCP (clone SK7/Cat# 347344), CD27-PE (L128/340425), CD28-APC (CD28.2/559770), CD25-PE (M-A251/555432), CD64-APC (10.1/561189), CD32-APC (FLI8.26/559769), CD45RO-APC (UCHL1/340438), CCR7-FITC (150503/561271), CD33-PE (P67.6/347787), PD1-PE (MIH4/ 557946), Rat Anti-Mouse IgG1-APC (X56/550874) (BD Biosciences, San Jose, Calif.), CD4-APC (13B8.2/IM2468U), CD4-Krome Orange (13B8.2/A96417), CD8-Pacific Blue (B9.11/A82791), CD8-PC7 (SFCI21Thy2D3/6607102), CD16-APC-AlexaFluor750 (3 G8/A66330), CD3-APC-AlexaFluor750 (UCHT1/A66329), (Beckman Coulter Inc.), anti-PSCA (7F5/sc-80654), mouse IgG1 (sc-3877) (Santa Cruz Biotechnology. Inc., Dallas, Tex.). CAR molecules were detected using Goat anti-human F(ab′)2 antibody conjugated with AlexaFluor647 (109-606-097) (Jackson ImmunoResearch Laboratories, Inc., West Grove, Pa.). Cells were stained with antibody for 20 min at 4° C. All samples were acquired on Gallios™ Flow Cytometer (Beckman Coulter Inc., Brea, Calif.) and the data were analyzed by Kaluza® Flow Analysis Software (Beckman Coulter Inc.).

Intracellular staining—T cells were first fixed with formaldehyde solution (F1635, Sigma-Aldrich, St. Louis, Mo.) at final 1.5% concentration. After washing, cells were permeabilized with pre-chilled 100% methanol (Fisher Scientific, Pittsburgh, Pa.) for 15 min on ice and washed three times. For phospho-FACS, cells were stained with anti-CD247 (pY142)-AlexaFluor647 antibody (K25-407.369/558489) (BD Biosciences) for 60 min at room temperature in the dark. For cell cycle analysis, cells were stained with anti-ki67-AlexaFluor647 (Ki-67/350510) (BioLegend, San Diego, Calif.) and 7-AAD (BD Biosciences) for 30 min in the dark at room temperature.

⁵¹Chromium-release assay—The cytotoxicity and specificity of engineered T cells were evaluated by a 4-6 hrs ⁵¹Cr-release assay, as previously described (Anurathapan, et al., 2014).

Co-culture experiments—For co-culture experiments with Fcy receptor-expressing cells, T cells were co-cultured with Fcy receptor-expressing cells at 1:1 ratio with 2 mL of complete media in a 24-well plate for 3 days. After culturing, all cells were harvested and stained with anti-CD3, anti-CD4 and anti-CD8 antibodies for T cells; anti-CD33 antibody for monocyte/macrophage; and anti-CD16 antibody for NK cells. For co-culture experiments with tumor cells, 5×10⁴ T cells were co-cultured with 10×10⁴ DU145 transduced with GFP/FFluc with 4 mL of complete media in a 6-well plate for 6 days. For co-culture experiments using Transwell (pore size 5μm; Product# 3421, Corning Life Sciences, Corning, N.Y.), 0.1×10⁶ Capan-1 cells transduced with GFP/FFluc were cultured in the bottom well overnight, then 0.1×10⁶ P1.CAR T cells were put into insert with or without 0.1×10⁶ irradiated THP-1 cells. After 12 hrs culture, insert was removed and cells were cultured further 24 hrs. All cells were harvested and stained with anti-CD3 antibody for T cells. CountBright™ Absolute Counting Beads (C36950; Invitrogen, Eugene, Oreg.) were added (50 uL) to count cell number and 7-AAD was added to exclude dead cells, and then analyzed by flow cytometry. Acquisition was stopped by counting 5,000 beads.

Cytokine detection—To compare the spontaneous cytokine release from T cells, 1.0×10⁶ T cells were plated into a well in 24-well tissue culture plate with 2 mL of complete media and cultured for 24 hrs. Supernatants were collected and stored at −80° C. To measure the cytokine profile of T cells, the inventors used the MILLIPLEX MAP High Sensitivity Human Cytokine Magnetic Bead Panel Premixed—13 Plex—Immunology Multiplex Assay (Merck Millipore, Billerica, Mass.) according to manufacturer's instructions.

Microarray analysis—Total RNA was extracted from T cells cultured for different culture period using RNeasy Mini kit (QIAGEN, Valencia, Calif.) and quantified using NanoDrop 2000 (Thermo Fisher Scientific Inc., Waltham, Mass.). RNA expression profiling was performed using the GeneChip PrimeView Human Gene Expression Array (Affymetrix, Inc., Santa Clara, Calif.) by Genome Exploration USA (Memphis, Tenn.). Microarray was performed from 3 independent donors.

In vivo study—Capan-1 cells (5×10⁶/animal) were engrafted at the right flank subcutaneously into female NOD.Cg-Prkdc^{scid} I12rg^{tm1Wj1}/SzJ mice (NSG mice, 6-8 weeks old, The Jackson Laboratory). After 28 days, once tumors were established (>80 mm³, measured using calipers), mice were treated with 10×10⁶ engineered T cells labeled with GFP/FFluc intravenously. Tumor size was measured by using calipers and tumor volume was calculated by the following formula; tumor volume (mm³)=length×width×width/2. T cell migration and distribution was evaluated by bioluminescence images recorded twice a week using Lumina IVIS imaging system (Caliper Life Sciences Inc., Hopkinton, Mass.), and analyzed by Living image software. Single cell suspension of tumor cells engrafted into NSG mice were performed by following the previous publication (Rasheed, et al., 2010) with only a slight modification. Briefly, tumor cells were dissected from mice and minced and dissociated by incubating with 200 U/mL collagenase IV (Gibco) 37° C. for 2 hours with voltexing for 1 min every 20 min. Tissue debris and dead cells were removed by density centrifugation using Lymphoprep (Axis-Shield, Oslo, Norway).

Glucose and Lactate measurement—Glucose and Lactate concentration in the T cell cultures was measured by ACCU-CHEK Aviva Plus system (Roche Diagnostics, Indianapolis, Ind.) and Lactate Plus (Nova Biomedical, Waltham, Mass.), respectively. Briefly, 20 uL of the supernatant was added on the sample loading area on ACCU-CHEK Active glucose test strip or Lactate test strip, which were mounted onto either ACCU-CHEK Aviva meter or Lactate Plus meter. Glucose and Lactate concentrations were calculated and reported as mg/dL and mM, respectively.

Statistics—Statistical analysis was performed using Graphpad Prism 6 software (GraphPad Software, Inc., La Jolla, Calif.). Two-way ANOVA was used for ⁵¹Cr-release assay and in vivo tumor growth. One-way ANOVA was used for the comparison of cytolytic function between different T cell ages. FDR corrected ANOVA was used for microarray analysis between different T cell ages from 3 independent donors. Unpaired two-tailed t-test was used for other experiments.

### Example 9

### Prediction of Tonic Signaling

The disclosure concerns methods for identifying the most favorable configuration of CAR construct having a balance between tonic signaling and effective antigen recognition.

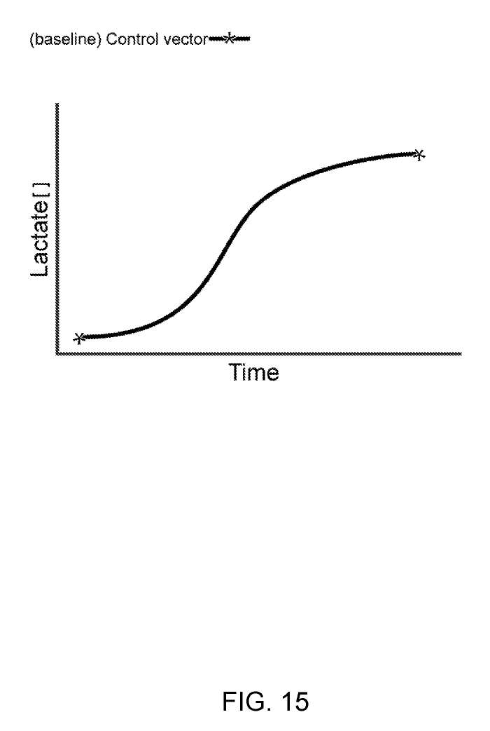

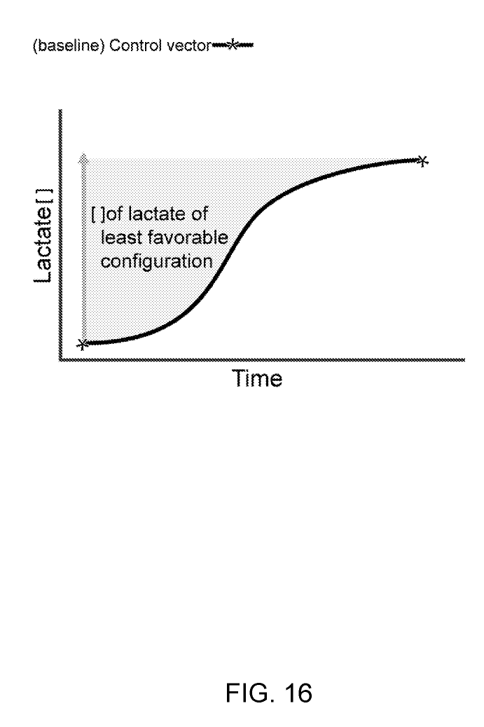

In the present example, aspects for measurement of tonic signaling are demonstrated. In particular embodiments, tonic signaling for a particular CAR is measured based on metabolic activity of cells that express the CAR. In specific cases, measurement of lactate concentration, glucose concentration, or a ratio thereof, taken as samples from supernatant from which the cells are cultured, is a metric for the amount of tonic signaling. For example in FIG. 15, it is illustrated how lactate concentration can be plotted over time to determine a baseline lactate production in a controlled vector devoid of tonic signal. Parameters for avoiding tonic signaling include one or more of the following: (i) a CAR without a signaling domain, (ii) a reporter molecule such as a fluorescent molecule, including GFP, (iii) a truncated marker such as CD19 or CD24, (iv) an empty vector, and (v) non-transduced cells. As shown in FIG. 16, once the lactate concentration (lactate, as an example) baseline has been identified (for example, conditions of T cell culture known to lack levels of tonic signaling), one can utilize the system to evaluate tonic signaling among different constructs and establish a hierarchy by identifying the one with the greatest tonic signaling as the configuration furthest away from the baseline.

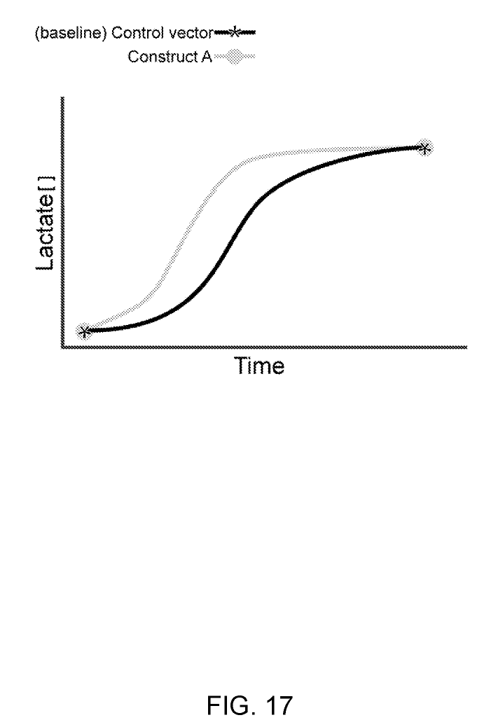

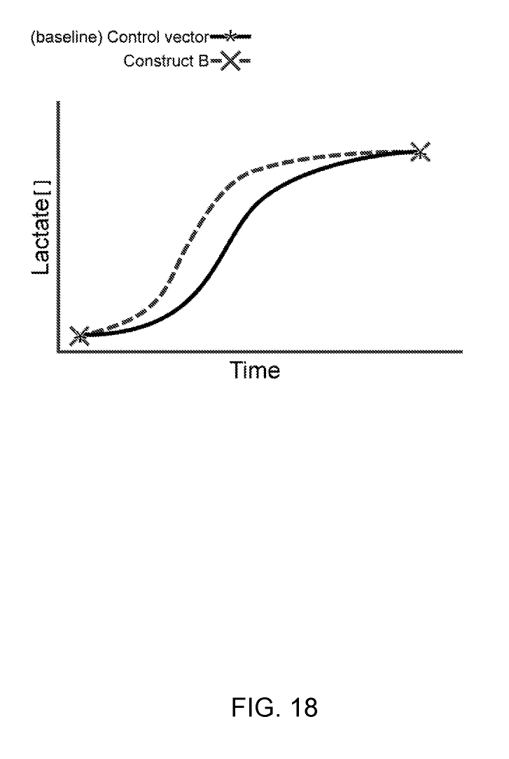

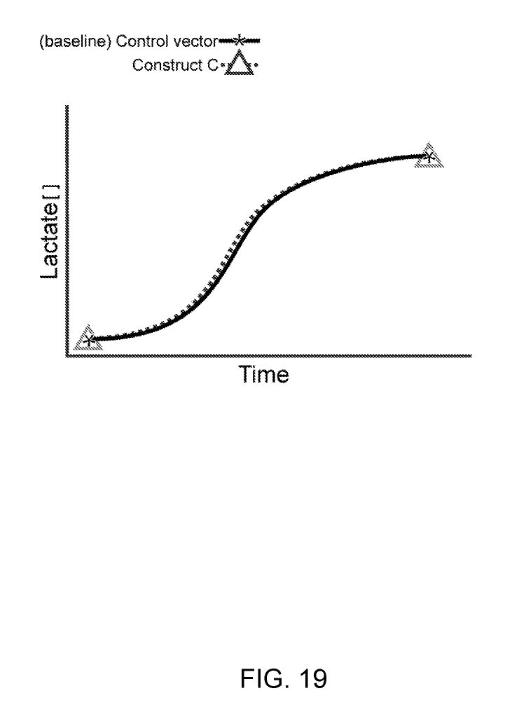

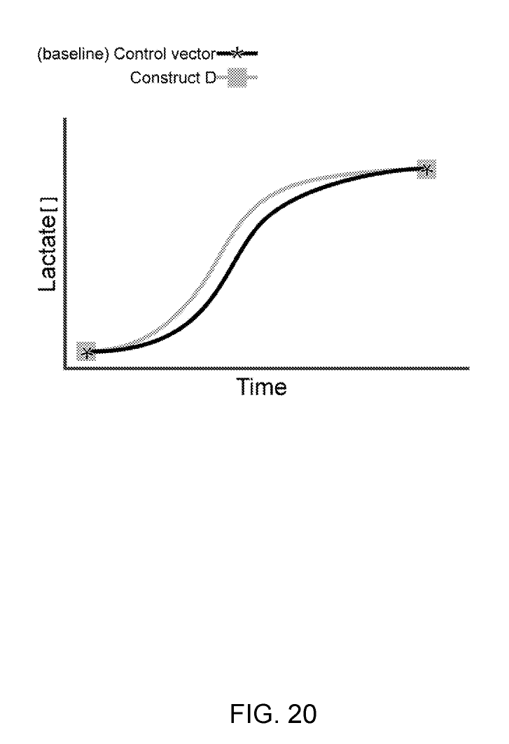

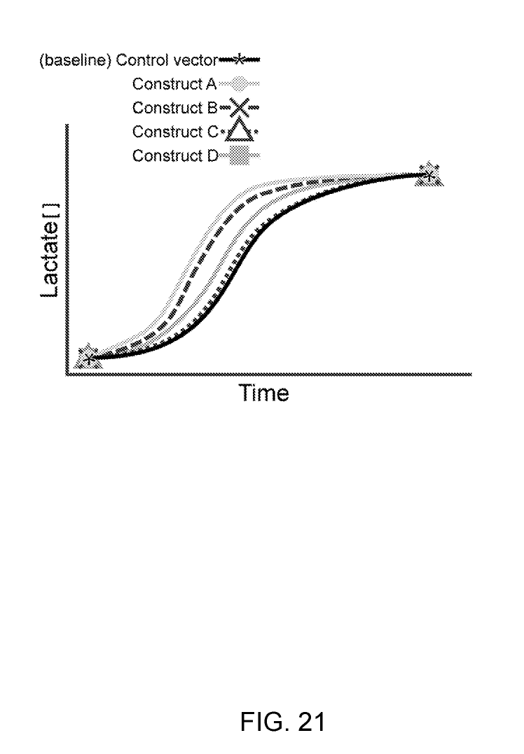

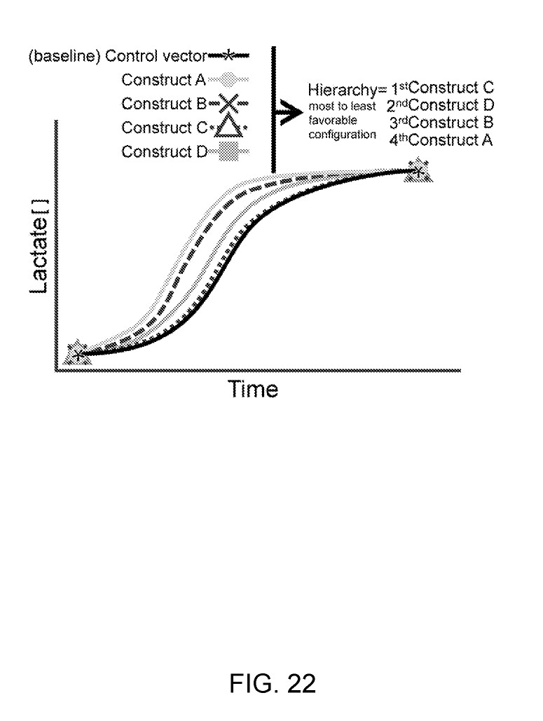

FIGS. 17-20 show the lactate concentration illustrated over time for a control vector that does not contain tonic signaling vs. one of exemplary Constructs A, B, C, or D, respectively. FIG. 21 compiles the results, and in FIG. 22 by comparing the lactate concentration among these different constructs, one can observe Construct C as closest to the baseline, indicating that this one will be the lowest with tonic signaling, followed by Construct D. This comparison can then be used to establish a hierarchy of tonic signaling where the most favorable configuration will be identified as the one closest to the baseline.

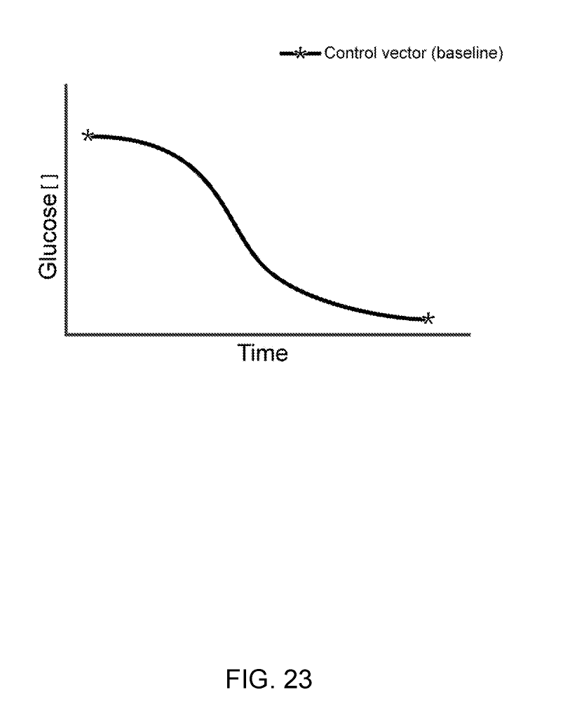

FIG. 23 illustrates how glucose concentration can be plotted over time to determine a baseline glucose production in a controlled vector devoid of tonic signal such as: (i) a CAR without a signaling domain, (ii) a reporter molecule, including a fluorescent molecule such as GFP, (iii) a truncated marker such as CD19 or CD24, (iv) an empty vector, and (v) non- transduced cells. Once the glucose concentration baseline has been identified (for example, by a T cell culture condition known to lack levels of tonic signaling), one can then evaluate the tonic signaling among different constructs and establish a hierarchy by identifying the one with the greatest tonic signaling as the configuration furthest away from the baseline.

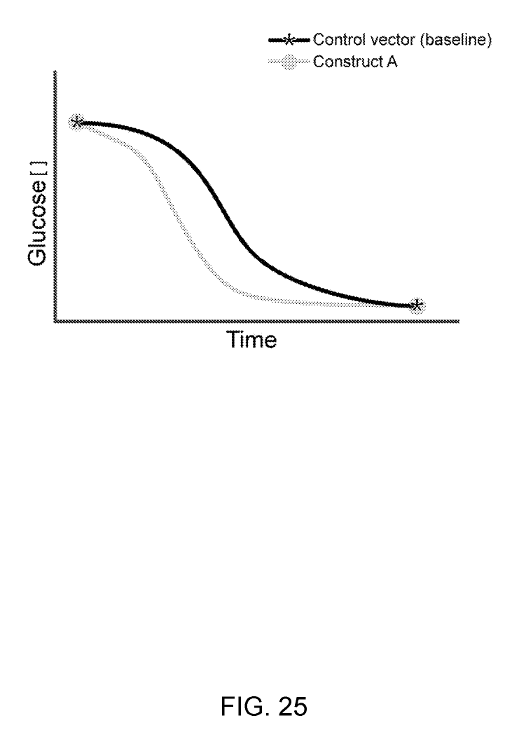

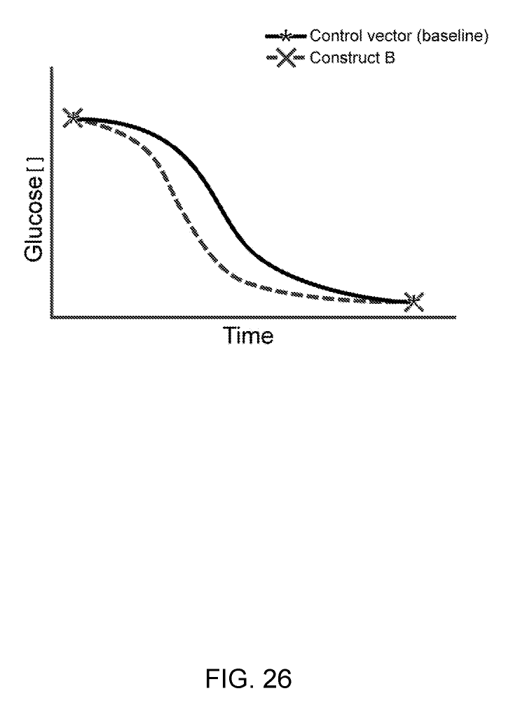

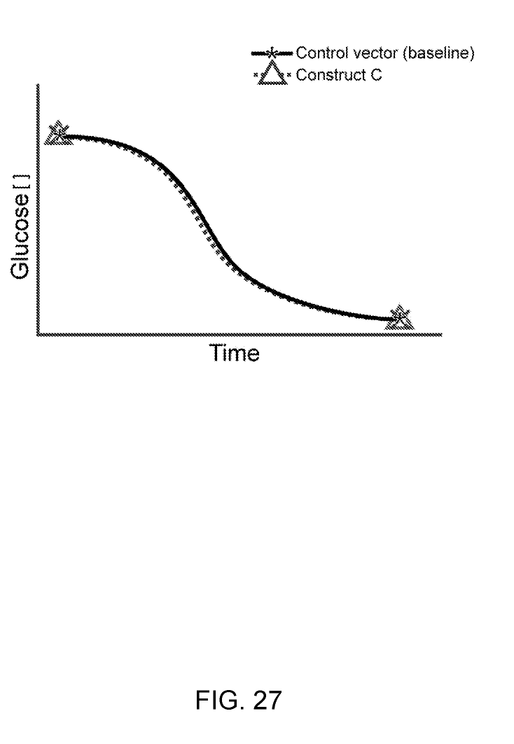

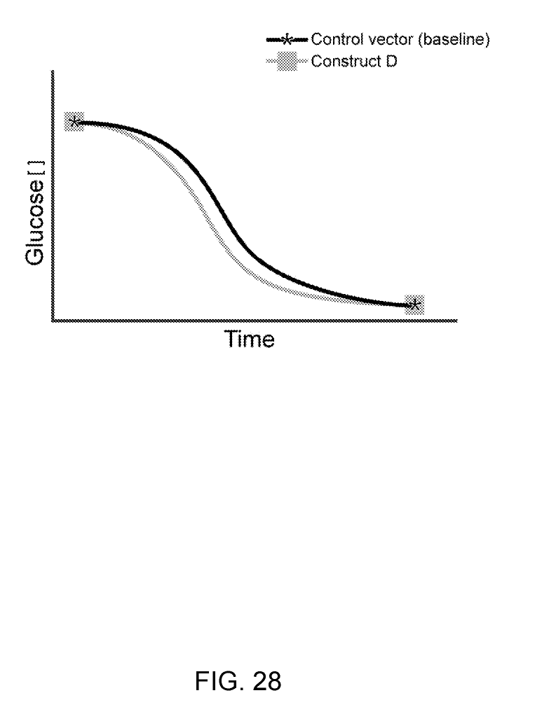

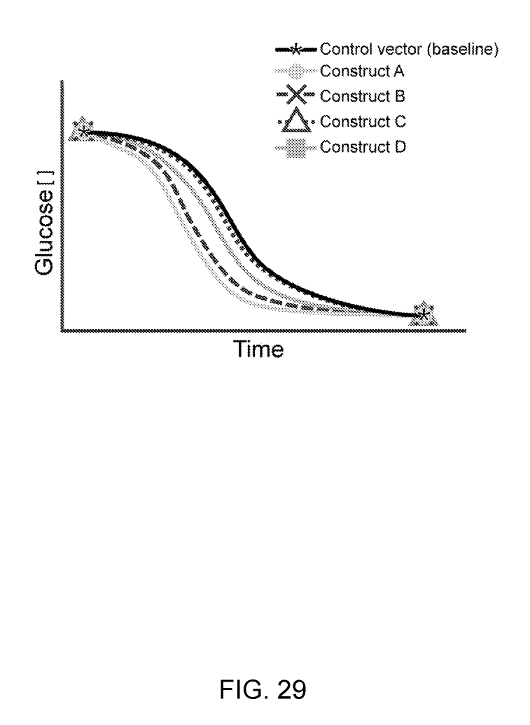

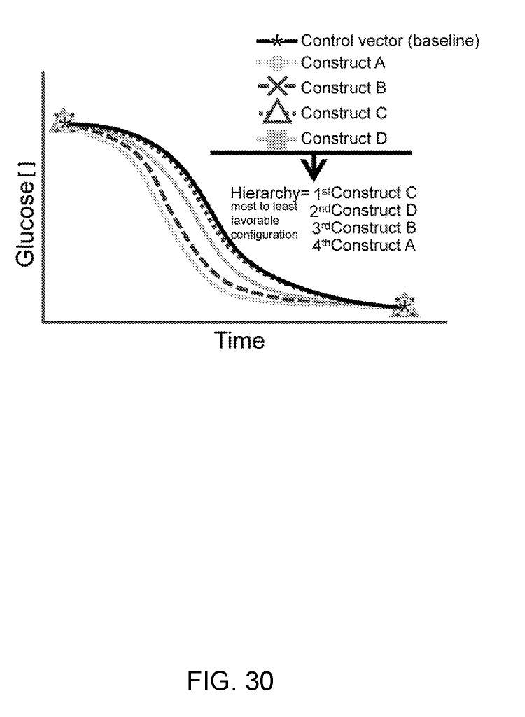

FIGS. 25-28 show glucose concentration illustrated over time with a control vector that does not contain tonic signaling vs. the exemplary constructs A, B, C, and D, respectively. Shown in FIG. 29, glucose concentration is illustrated over time of for these multiple constructs vs. the Control vector that does not contain tonic signaling. In FIG. 30, by comparing the glucose concentration among these different constructs, one can observe Construct C as closest to the baseline, indicating that this one will be the lowest with tonic signaling, followed by Construct D. This comparison can then be used to establish a hierarchy of tonic signaling where the most favorable configuration will be identified as the one closest to the baseline.

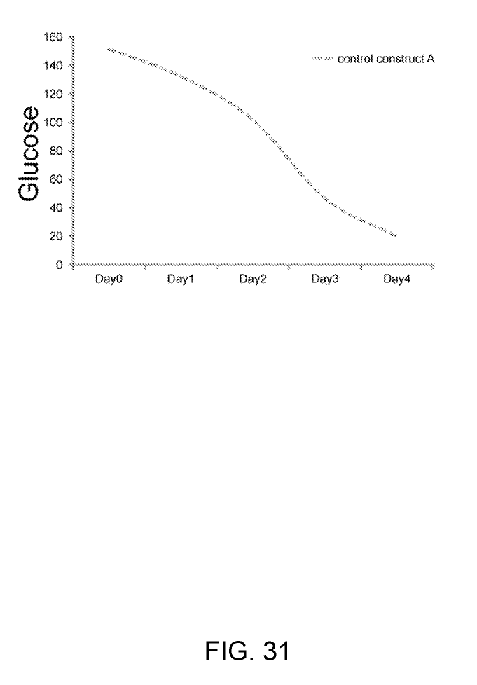

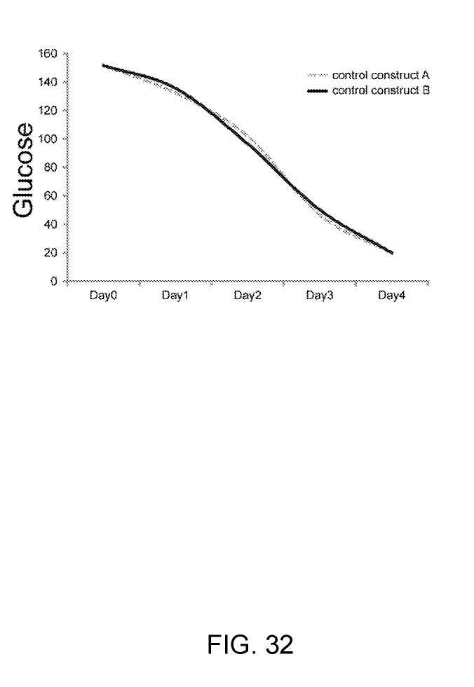

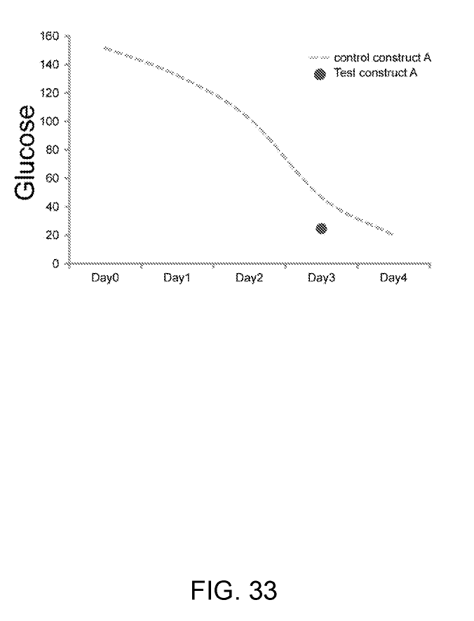

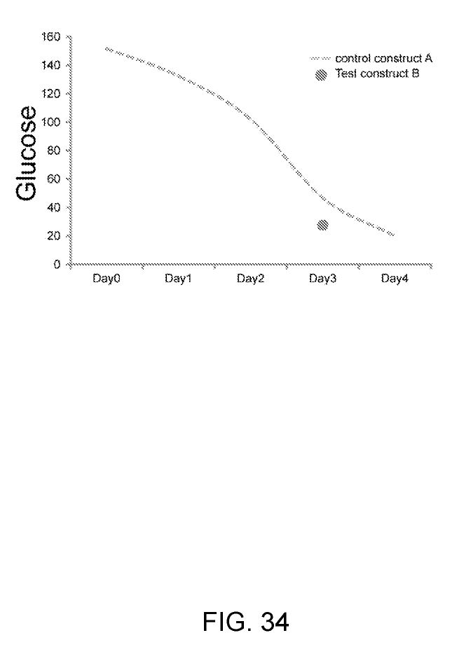

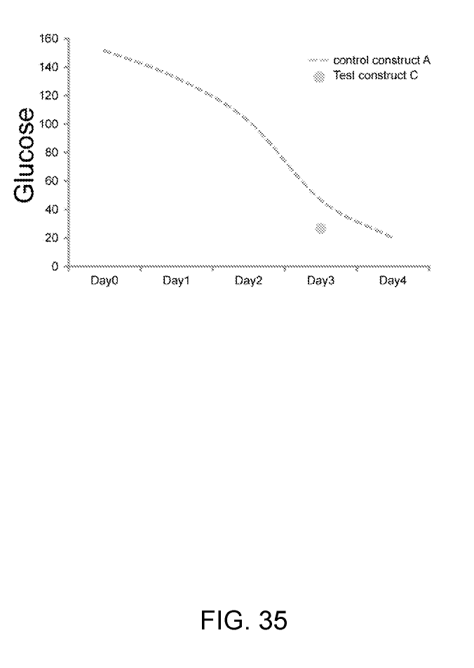

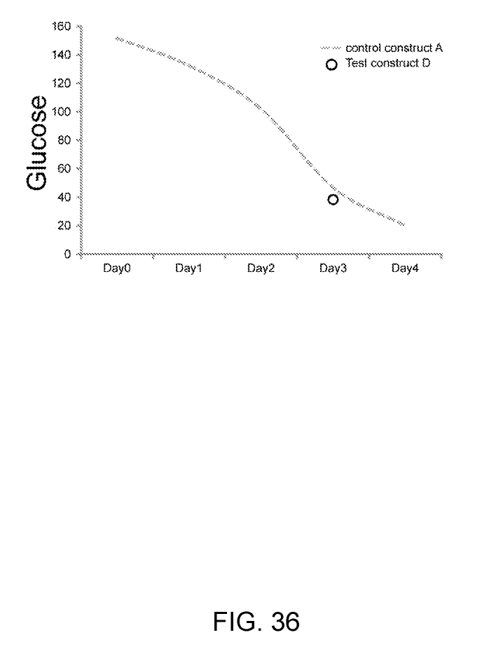

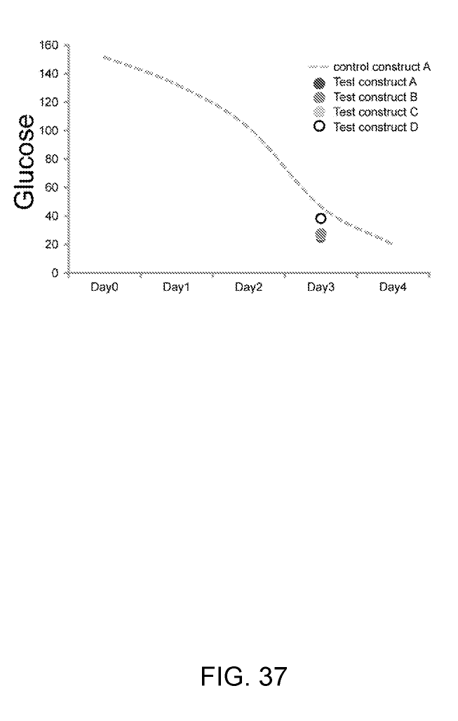

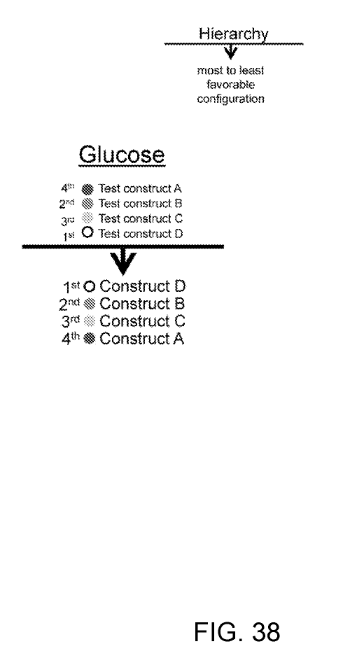

The following figures and description concern actual data sets. FIGS. 31-38 utilize glucose concentration as a parameter for measurement. In FIG. 31, as an example, Construct A illustrates the pattern of glucose consumption of T cells expressing a truncated CAR-PSCA that lacks the signaling endodomain (glucose consumption baseline). In FIG. 32, it is illustrated how the baseline of glucose consumption can be obtained by using a CAR-lacking endodomain (Construct A), and comparing this with T cells that are non-transduced (Construct B). Therefore, either Control A or B can be used to establish the baseline. FIG. 33 illustrates the glucose concentration of the control construct A and the glucose concentration of Test construct A when measured at Day 3 of the culture. FIG. 34 demonstrates the glucose concentration of the control construct A and the glucose concentration of Test construct B when measured at Day 3 of the culture. FIG. 35 shows the glucose concentration of the control construct A and the glucose concentration of Test construct C when measured at Day 3 of the culture. FIG. 36 illustrates the glucose concentration of the control construct A and the glucose concentration of Test construct D when measured at Day 3 of the culture. Thus, in FIG. 37, the glucose concentration of multiple test conditions is compared as long as the same time set has been acquired for all test conditions. This example also illustrates how a single time assessment is sufficient to make this comparison. As shown therein, construct D has the lowest tonic signaling as this is closest to the baseline. FIG. 38 demonstrates that based on the difference in glucose concentration, one can establish a hierarchy where, in this case, the most favorable configuration is the one with the lowest tonic signaling.

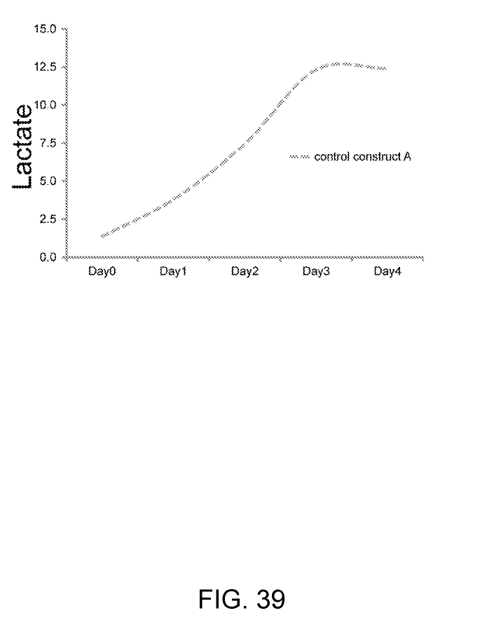

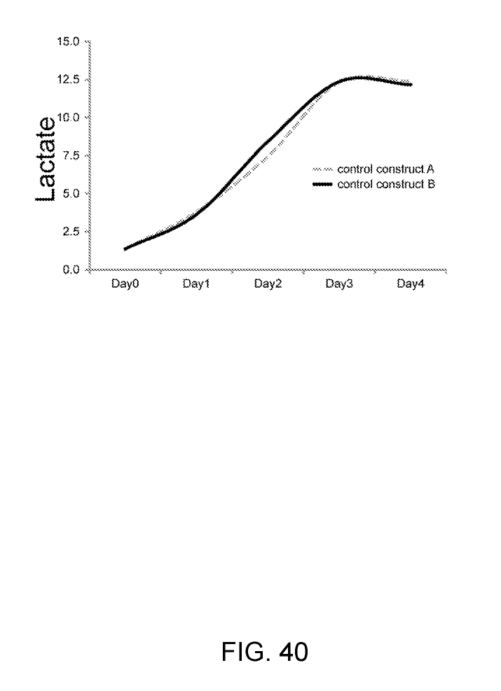

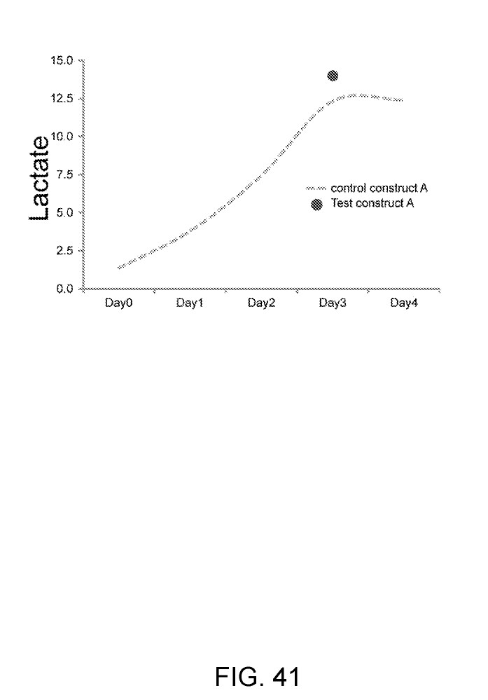

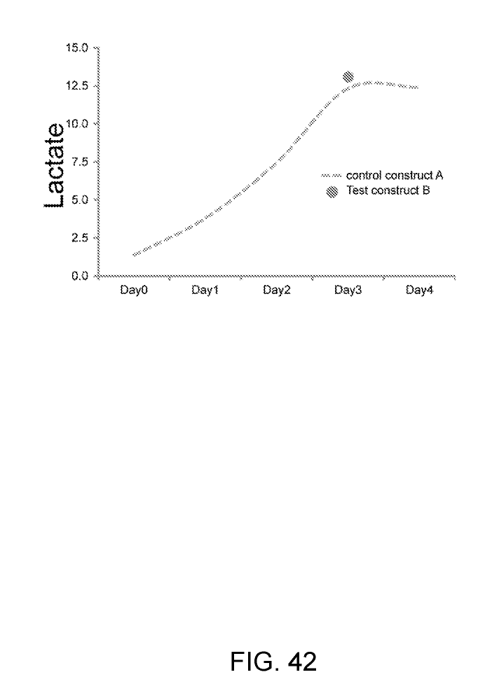

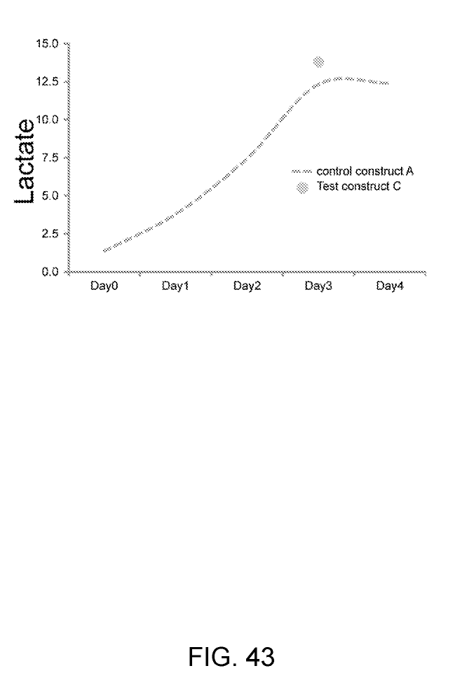

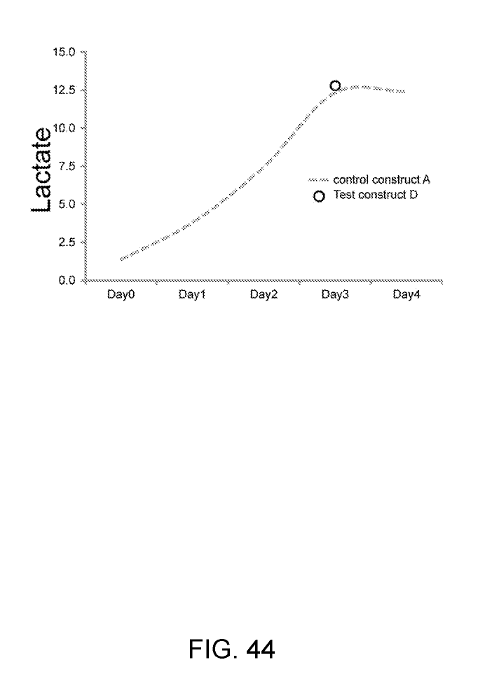

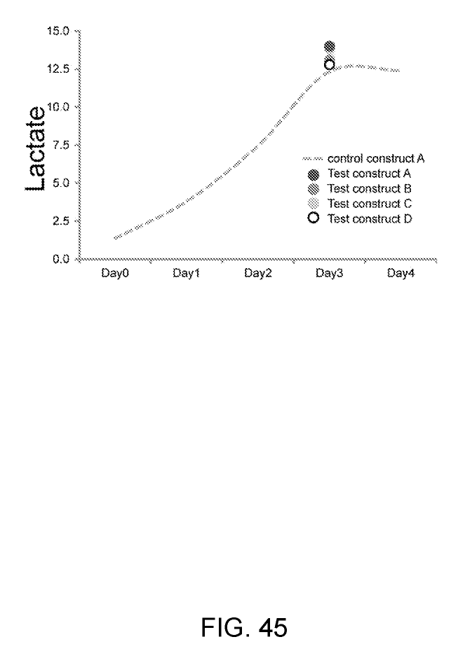

FIGS. 39-45 utilize lactate concentration as a parameter for measurement. In FIG. 39, Construct A illustrates the pattern of lactate consumption of T cells expressing a truncated CAR-PSCA (as an example) that lacks the signaling endodomain (lactate consumption baseline). FIG. 40 illustrates how the baseline of lactate consumption can be obtained by using a CAR-lacking endodomain (Construct A), and comparing this with T cells that are non-transduced (Construct B). Therefore, either Control A or B can be used to establish the baseline. FIG. 41 shows the lactate concentration of the control construct A and the lactate concentration of Test construct A when measured at Day 3 of the culture. FIG. 42 illustrates the lactate concentration of the control construct A and the lactate concentration of Test construct B when measured at Day 3 of the culture. FIG. 43 demonstrates the lactate concentration of the control construct A and the lactate concentration of Test construct C when measured at Day 3 of the culture. In FIG. 44, the lactate concentration of the control construct A and the lactate concentration of Test construct D when measured at Day 3 of the culture are shown. In FIG. 45, the lactate concentration of multiple test conditions are compared as long as the same time set has been acquired for all test conditions. This example also illustrates how a single time assessment is sufficient to make this comparison. As shown therein, construct D has the lowest tonic signaling, because it is closest to the baseline.

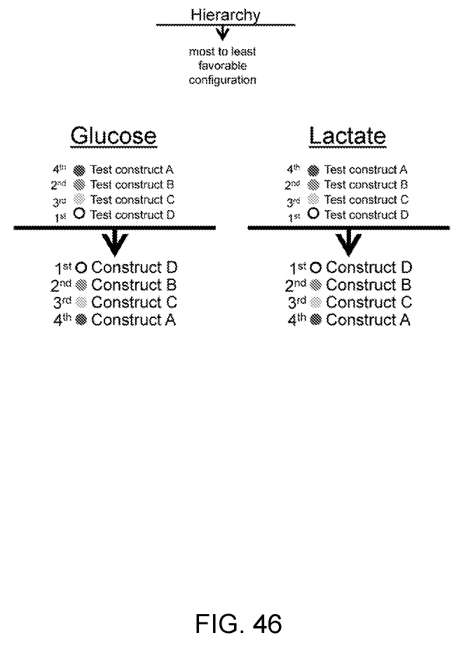

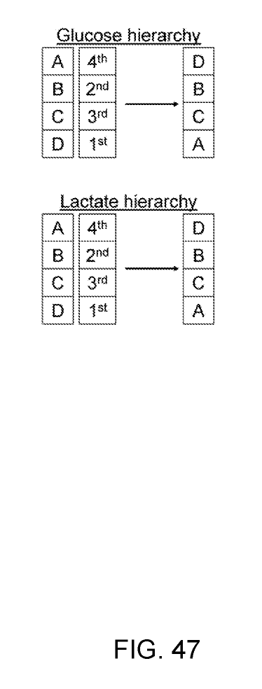

Based on the difference in glucose and lactate concentration, one can establish a hierarchy where in this case, the most favorable configuration is the one with the lowest tonic signaling (FIG. 46). Therefore, the concentration of glucose and lactate collected from the media of T cells expressing these different constructs can be used to establish a hierarchy of tonic signaling (FIG. 47).

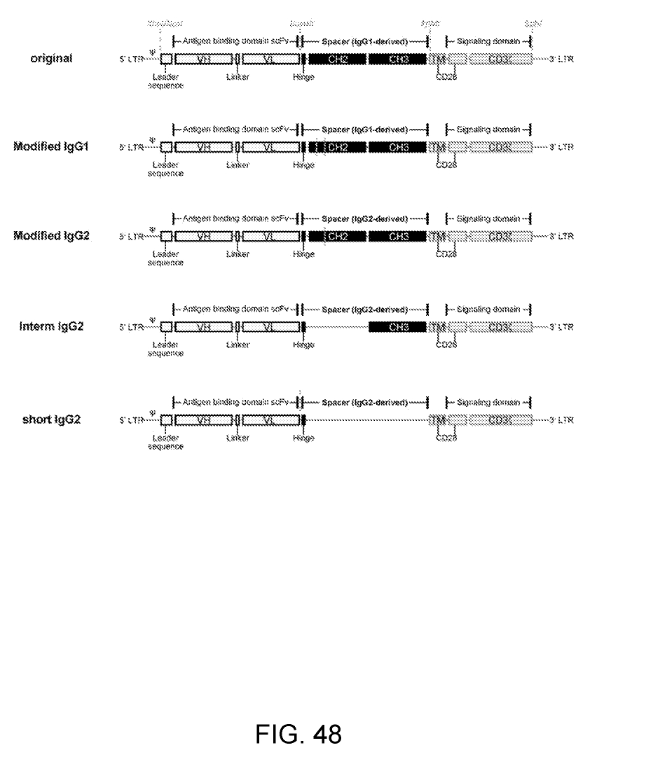

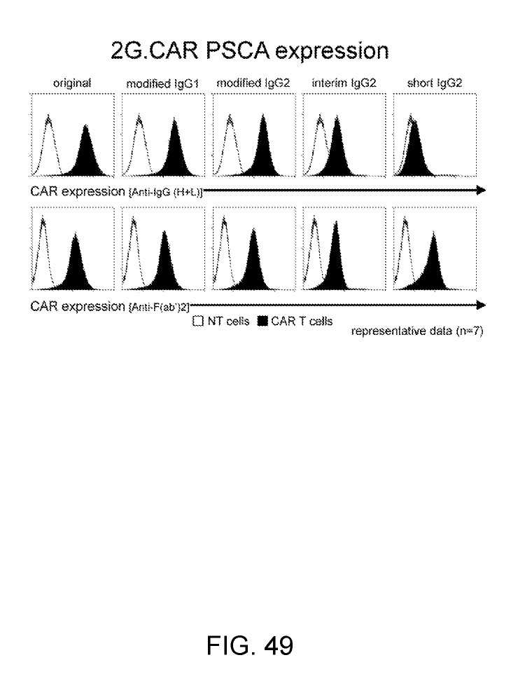

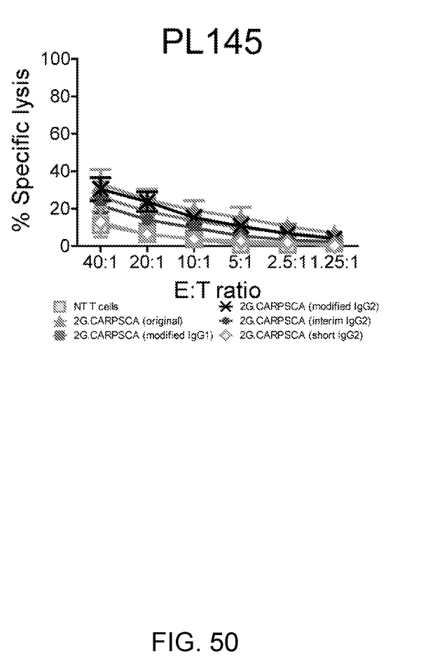

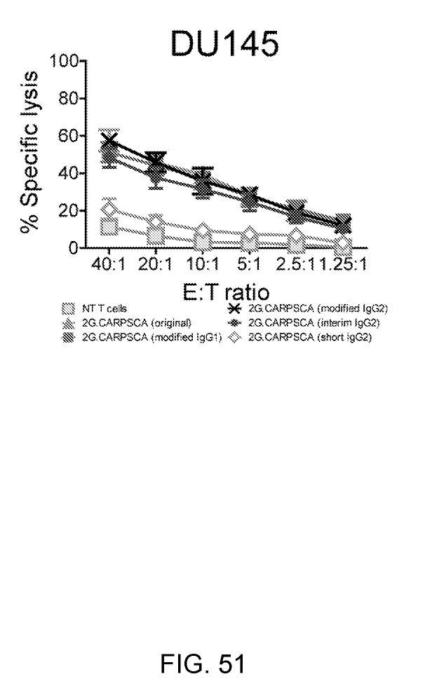

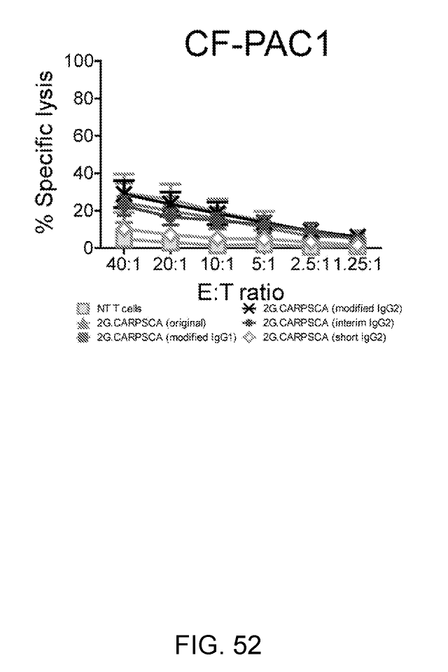

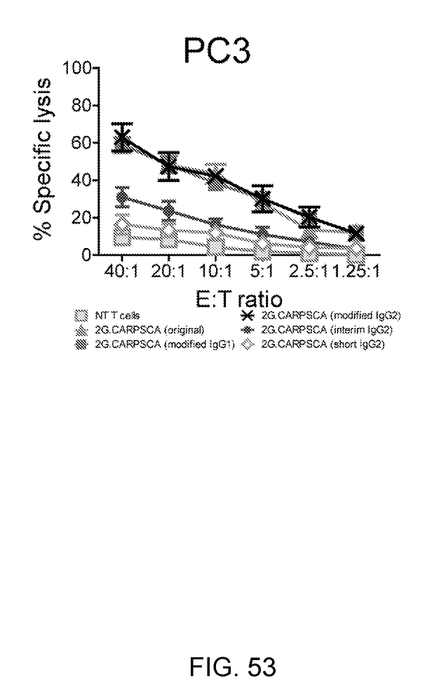

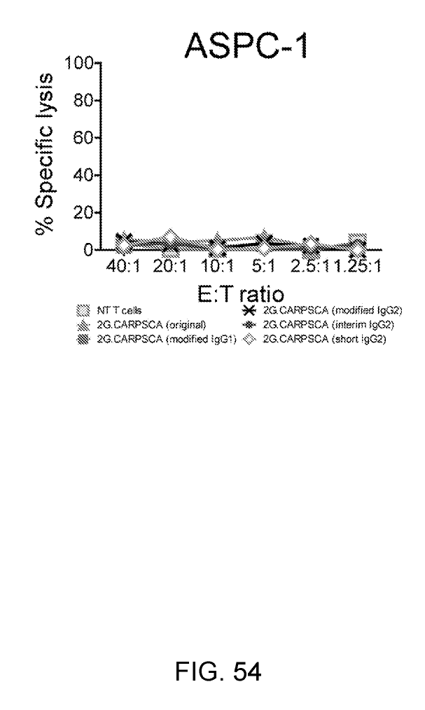

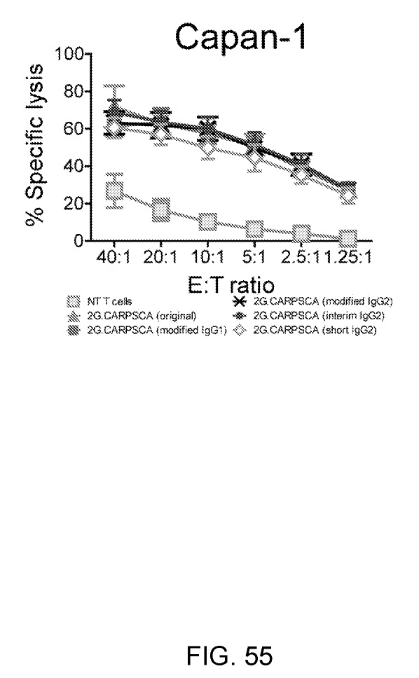

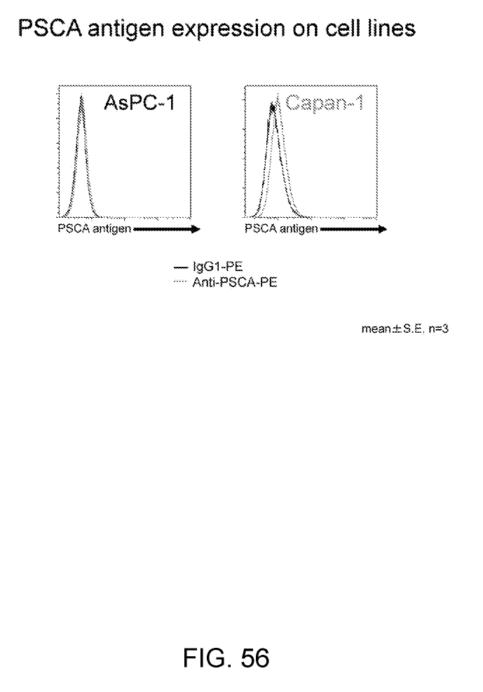

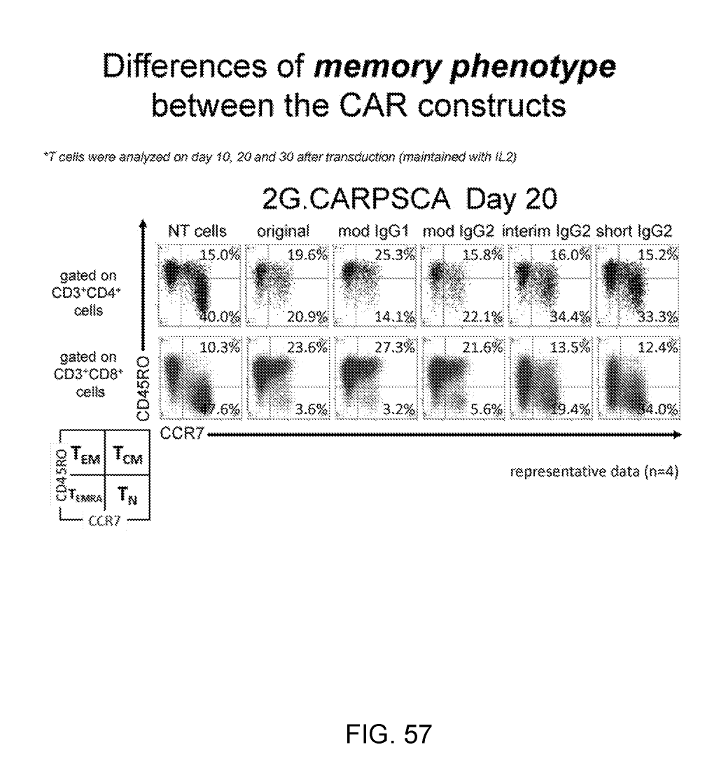

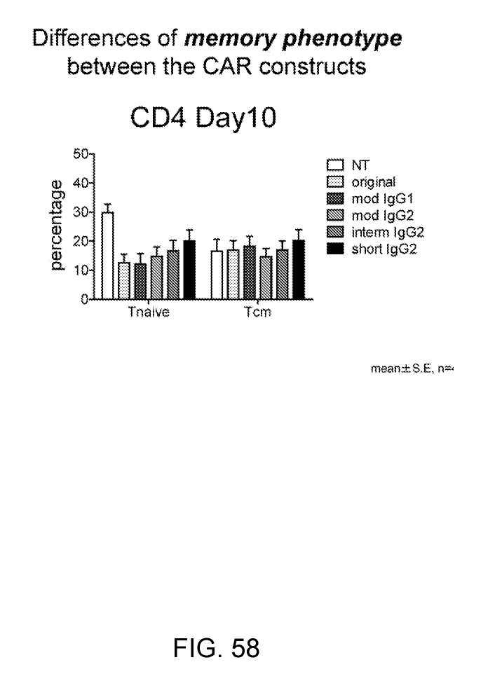

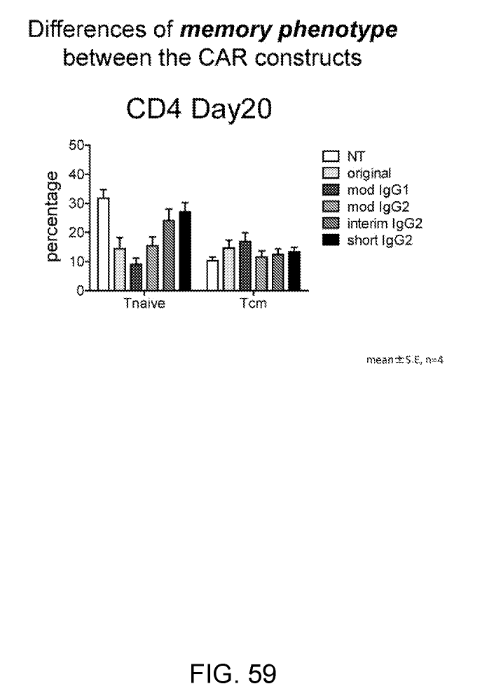

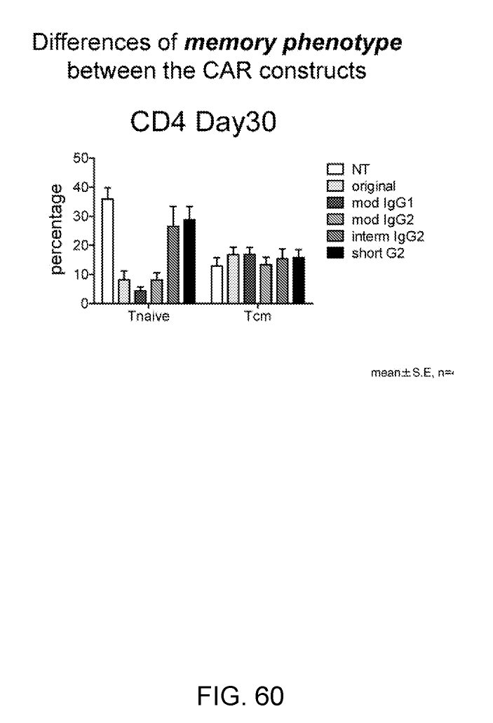

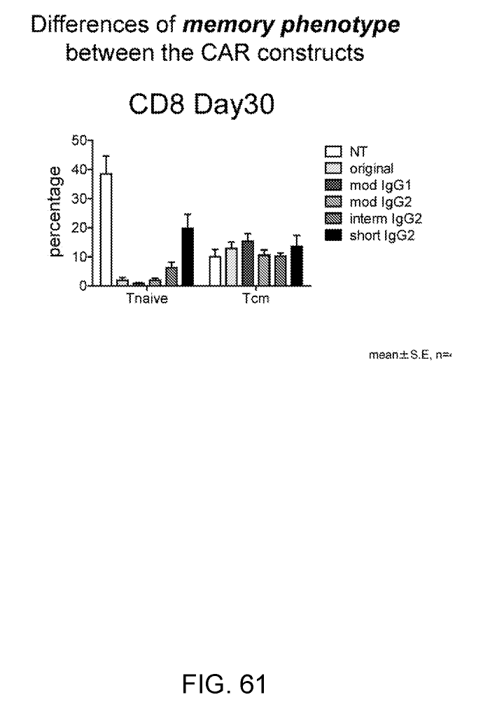

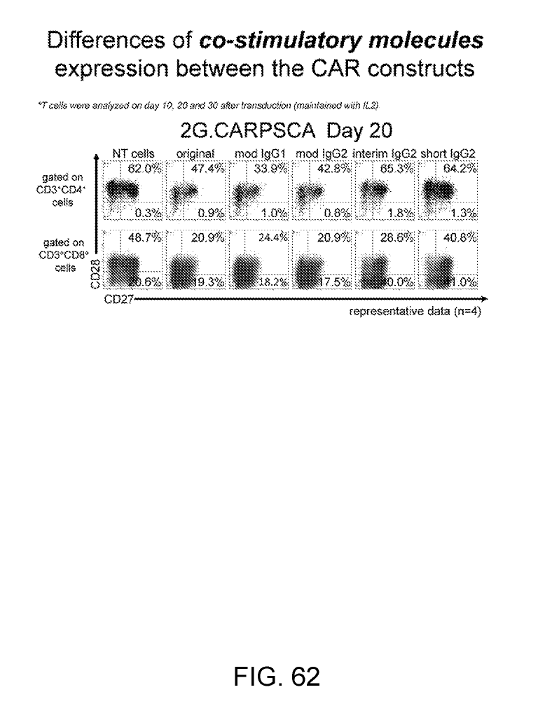

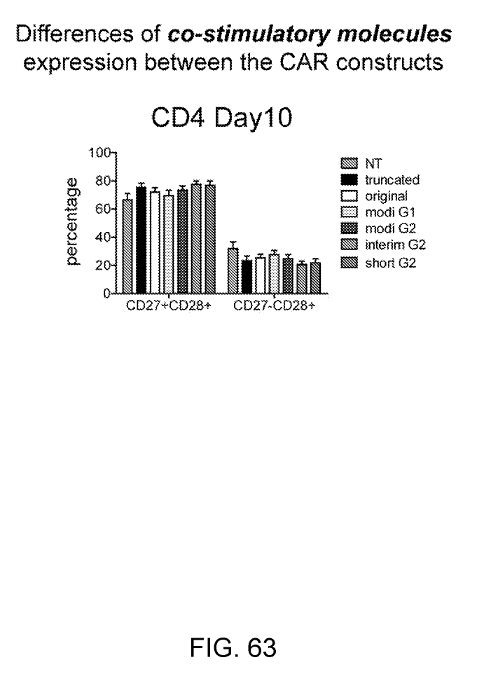

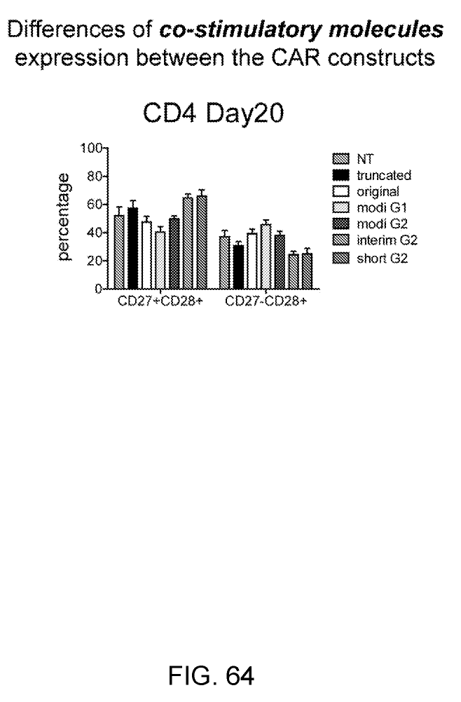

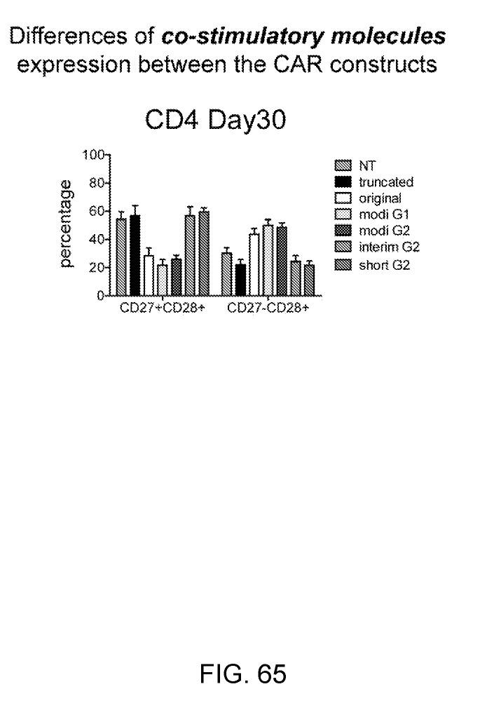

The impact of CAR spacer configurations with antigen recognition and T cell phenotype is shown in FIGS. 48-65. For example, FIG. 48 illustrates an example of a vector map of CAR constructs containing various spacer length. In FIG. 49, the CAR expression of T cells after retroviral transduction is shown. The upper panel shows the staining used in an anti-IgG antibody, as expected the “short IgG2 CAR” is not stained as this molecule does not contain CH2CH3. In the lower panel, this illustrates the CAR expression using an anti-F(ab′)2 antibody, in this condition all the molecules are detected. FIGS. 50 and 51 show the killing of CARs with different lengths of spacers. FIG. 51 demonstrates the killing of CARs with different lengths of spacers (DU145 cell lines). When targeting tumor cells that express intermediate levels of antigen expression, the CAR with the short spacer resulted in reduced antigen recognition properties. FIG. 52 also shows the killing of CARs with different lengths of spacers (CF-PAC1 cell lines). FIG. 53 demonstrates the killing of CARs with different lengths of spacers (PC3 cell lines). When targeting tumor cells that express low levels of antigen expression the CAR with the short and intermediate spacer resulted in reduced antigen recognition properties. FIG. 54 shows the killing of CARs with different lengths of spacers (ASPC-1 cells). FIG. 55 demonstrates the killing of CARs with different lengths of spacers (Capan-1 cells). When targeting tumor cells that express high levels of antigen expression the CAR with a long, intermediate, or short spacer resulted in similar killing properties. FIG. 56 shows the antigen expression (PSCA) on two different cancer cells lines. FIG. 57 demonstrates the memory profile of T cells transduced with different CAR constructs after culture for 20 days in media with IL2 in absence of antigen stimulation. FIG. 58 shows the naive phenotype versus the central memory phenotype of CD4 T cells, transduced with different CAR constructs, at 10 days of culture. FIG. 59 demonstrates at 20 days of culture the naive phenotype versus the central memory phenotype of CD4 T cells, transduced with different CAR constructs. FIG. 60 shows the naive phenotype versus the central memory phenotype of CD4 T cells, transduced with different CAR constructs, at 30 days of culture. FIG. 61 shows the naive phenotype versus the central memory phenotype of CD8 T cells, transduced with different CAR constructs, at 30 days of culture. FIG. 62 demonstrates the differences of co-stimulatory molecules (CD27/CD28) profile of T cells transduced with different CAR constructs after culture for 20 days in media with IL2 in absence of antigen stimulation. FIG. 63 shows the double positive CD27/CD28 population and single CD28 population on CD4 T cells transduced on different CAR configurations at Day 10 of culture. FIG. 64 demonstrates the double positive CD27/CD28 population and single CD28 population on CD4 T cells transduced on different CAR configurations at Day 20 of culture. FIG. 65 shows the double positive CD27/CD28 population and single CD28 population on CD4 T cells transduced on different CAR configurations at Day 30 of culture.

A vector map of examples of CAR constructs is provided in FIG. 48. FIG. 49 illustrates the CAR expression of T cells, in this case after retroviral transduction. The upper panel shows the staining used in an anti-IgG antibody, as expected the “short IgG2 CAR” is not stained because this molecule does not contain CH2CH3. In the lower panel, CAR expression is illustrated using an anti-F(ab′)2 antibody, and in this condition all of the molecules are detected. FIGS. 50-55 illustrate the killing by CARs with different lengths of spacers (where E:T is the Effector Cell to Target Cell ratio) for PL145 cells (FIG. 50), DU145 cells (FIG. 51), CF-PAC1 cells (FIG. 52), PC3 cells (FIG. 53), ASPC-1 cells (FIG. 54), and Capan-1 cells (FIG. 55).

As in FIG. 51, in specific embodiments, when targeting tumor cells that express intermediate levels of antigen expression, the CAR with the short spacer resulted in reduced antigen recognition properties. As in FIG. 53, in specific embodiments, when targeting tumor cells that express low levels of antigen expression the CAR with the short and intermediate spacer resulted in reduced antigen recognition properties. As in FIG. 55, in specific embodiments, when targeting tumor cells that express high levels of antigen expression the CAR with a long, intermediate, or short spacer resulted in similar killing properties. FIG. 56 shows the antigen expression (PSCA) on two different examples of cancer cells lines.

FIG. 57 demonstrates the memory profile of T cells transduced with different CAR constructs after culture for 20 days in media with IL2 in absence of antigen stimulation. As illustrated therein, the memory profile of both CD4 and CD8 cells have a larger proportion of naive T cells, while in contrast, CAR T cells transduced with different constructs can have a direct effect on the memory profile of the T cells. In a specific embodiment, constructs with a greater amount of tonic signaling tend to lose the naive phenotype and develop into a terminally differentiated population. In contrast, constructs with decreased levels of tonic signaling have a greater proportion of naive T cells in culture. These are features that are correlated with greater in vivo function, in particular aspects. In FIG. 58, the naive phenotype versus the central memory phenotype of CD4 T cells, transduced with different CAR constructs, at 10 days of culture is demonstrated. In FIG. 59, the naive phenotype versus the central memory phenotype of CD4 T cells, transduced with different CAR constructs, at 20 days of culture is demonstrated. In FIG. 60, the naive phenotype versus the central memory phenotype of CD4 T cells, transduced with different CAR constructs, at 30 days of culture is shown. FIG. 61 demonstrates the naive phenotype versus the central memory phenotype of CD8 T cells, transduced with different CAR constructs, at 30 days of culture.

FIG. 62 shows the differences of a co-stimulatory molecules (CD27/CD28) profile of T cells transduced with different CAR constructs after culture for 20 days in media with IL2 in absence of antigen stimulation. As shown therein, the double population of CD27 and CD28 cells was greatest in the non-transduced T cells, followed by CAR configurations with the least amount of tonic signaling. In specific embodiments, constructs with a greater amount of tonic signaling had the least amount of double negative CD27 and CD28 population. In contrast, constructs with decreased levels of tonic signaling have a greater proportion of double positive cells (CD28/CD27) in culture. These are features that are correlated with greater in vivo function, in particular embodiments.

The double positive CD27/CD28 population and single CD28 population on CD4 T cells transduced on different CAR configurations at Day 10 of culture (FIG. 63), Day 20 of culture (FIG. 64), and Day 30 of culture (FIG. 65).

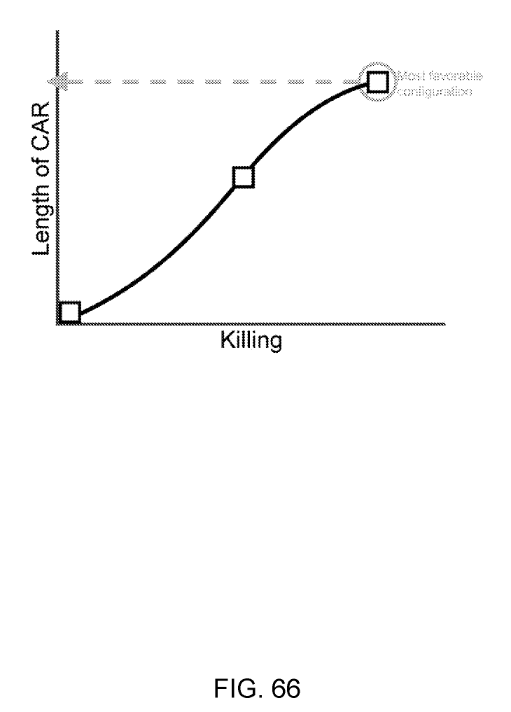

FIGS. 66-69 demonstrates the identification of optimal CAR configurations. FIG. 66 illustrates the current knowledge based on what is known in the art. In this schematic representation, the X-axis represents the killing ability of T cells (where “killing” refers to shorter in vitro interaction as illustrated by a 4 hour chromium release assay) this can be considered as a magnitude of antigen recognition. The Y-axis represents the length of the CAR spacer. In this particular example, a CAR configuration with a long spacer has a direct correlation with the antigen recognition properties assessed by an in vitro killing assay (different measurements of this interaction are not limited to killing; they can be extended to other biological properties such as cytokine production, interferon gamma, IL2, TNF). Therefore, based on this relationship one could predict a long spacer will result with a better in vivo antitumor activity.

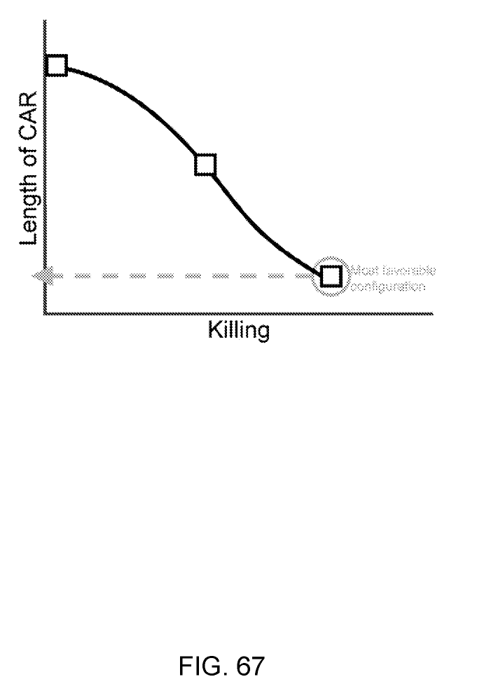

FIG. 67 illustrates the current knowledge based on what is known in the art. In this schematic representation, the X-axis represents the killing ability of T cells (where “killing” refers to shorter in vitro interaction, such as being illustrated by a 4 hour chromium release assay), and this can be considered as a magnitude of antigen recognition. The Y-axis represents the length of the CAR spacer. In this particular example, a CAR configuration with a long spacer has an indirect correlation with the antigen recognition properties assessed by an in vitro killing assay (different measurements of this interaction are not limited to killing; they can be extended to other biological properties such as cytokine production, interferon gamma, IL2, TNF). Therefore, based on this relationship one could predict a short spacer will result with a better in vivo antitumor activity. In one aspect, the discrepancy between FIGS. 66 and 67 is related to the location of the epitope within the antigen. Therefore, in specific embodiments when the epitope is proximal to the target cell membrane, the scenario in FIG. 66 is appropriate; while in contrast, when the epitope is exposed/distant to the antigen, the scenario in FIG. 67 is more likely to occur.

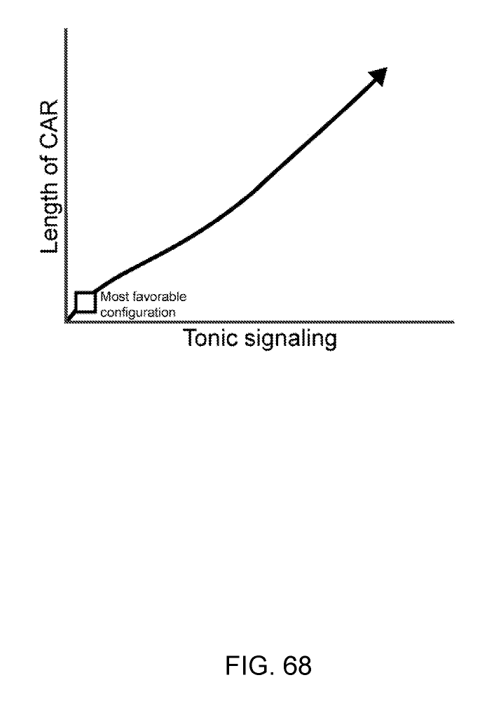

FIG. 68 demonstrates a novel, direct correlation between the CAR spacer and tonic signaling. Therefore, in some embodiments the most desired configuration is one with the least amount of tonic signaling, because high levels of tonic signaling can be correlated with an unfavorable T cell phenotype and limited in vivo T cell persistence. Therefore, in some embodiments if one only considers the most favorable CAR configuration based on tonic signaling, one would select a CAR construct with a shorter spacer.

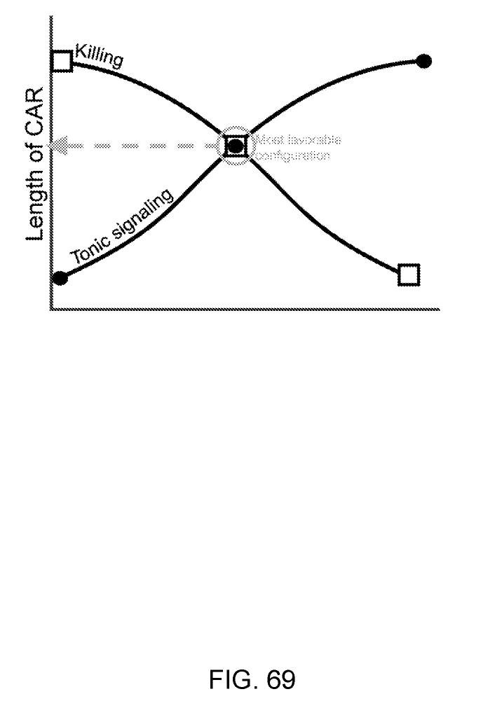

However, FIG. 69 takes into consideration two components: (i) antigen recognition (previously known to be related with the length of the CAR), and (ii) tonic signaling. In particular embodiments, a favorable configuration regarding the length of the CAR is one that has both of these components. Particularly, in the case for CAR-PSCA (merely as an example), the antigen recognition (in vitro killing) was best when using a long CAR spacer, but the tonic signaling was the lowest when using the shorter spacer. Therefore, by taking these two parameters into consideration, an intermediate CAR provides adequate antigen recognition and relatively low levels of tonic signaling—resulting in an improved antitumor in vivo activity. These are aspects that would have been unexpected by a person skilled in the art.

### Example 10

### Chimeric FC Receptor Target Molecules and Uses Thereof

As described herein, in at least some cases part of an IgG Fc domain (for example, the CH2CH3 hinge region) that is a component of an engineered receptor would facilitate the binding of cells that express that receptor to cells that express a Fc-gamma receptor. Such binding is detrimental when the engineered receptor is to be utilized for T cell-mediated killing (including a chimeric antigen receptor, for example). However, in cases wherein it is desirable for the receptor expressing-T cells to bind Fc-gamma receptor-bearing cells (cells such as monocytes, macrophages, dendritic cells, neutrophils, and so forth), this mechanism may be exploited.

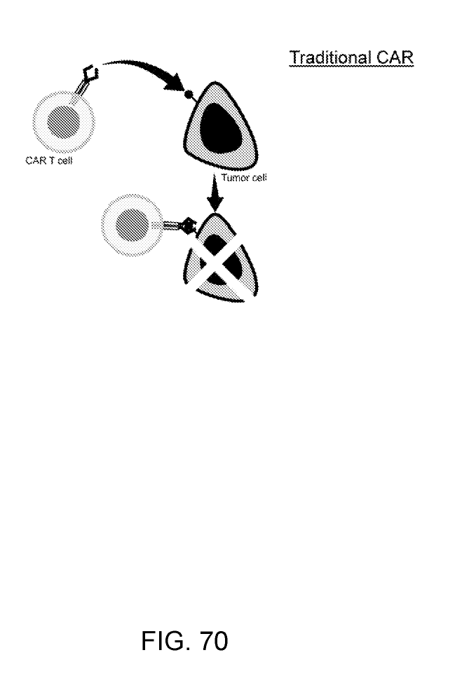

That is, FIG. 70 illustrates a traditional CAR that functions by the recognition of an antigen that is expressed on target cells, allowing T cell-mediated killing. The present example describes embodiments that are the “reverse” of such an output. In cases wherein the engineered receptor on the cell is a full CAR molecule, the following embodiments may be considered to be reverse CARs. In cases wherein the engineered receptor on the cell lacks an scFv, the receptor may be considered to be a chimeric Fc receptor target molecule.

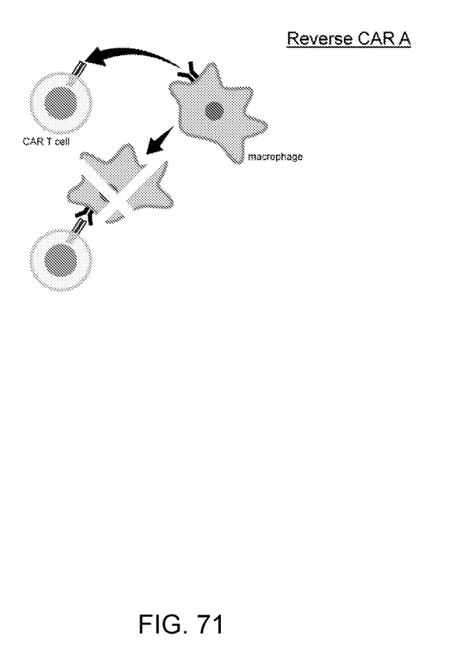

FIG. 71 illustrates one embodiment, wherein CAR T cells express a chimeric Fc receptor target molecule that comprises one or more FcγR-binding domains of an IgG Fc domain (such as the CH2CH3 region of an IgG). In specific embodiment, the chimeric Fc receptor target molecule comprises or lacks a scFv). As illustrated by the figure, the CH2CH3 region as an example allows for the recognition of Fc-gamma receptor-expressing cells, such as macrophages, resulting in the elimination of the Fc-gamma receptor-expressing cells. Therefore, by expressing a molecule that can be recognized by the target cell, one can induce the killing of the target cell itself.

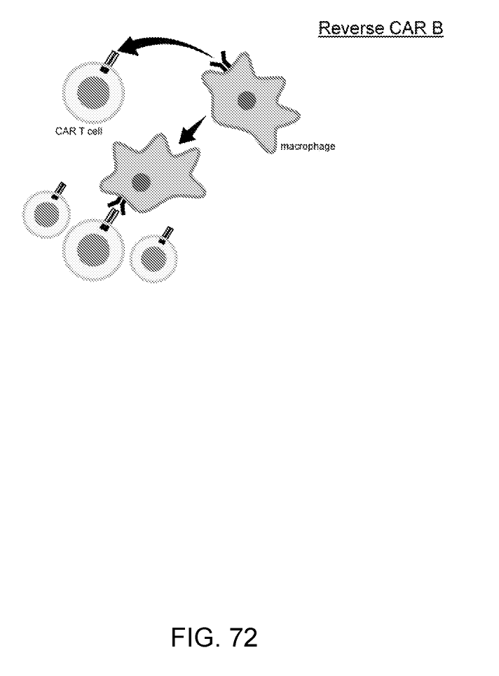

In FIG. 72, a specific embodiment of a reverse CAR is illustrated. In this case, target cells recognize a express a chimeric Fc receptor target molecule expressed by the T cells (for example, the CH2CH3 region of an IgG) while containing only co-stimulatory endodomains such as CD28. Therefore, once the T cells get recognized by the macrophages, this will induce dimerization of the molecule and T cell proliferation, but there is no killing of the Fc-gamma receptor-expressing cells, because the CD3zeta is not incorporated within the molecule. In specific embodiments, the purpose of such an embodiment includes increasing expansion of cells that bear the chimeric Fc receptor target molecule, such as T cells, for example.

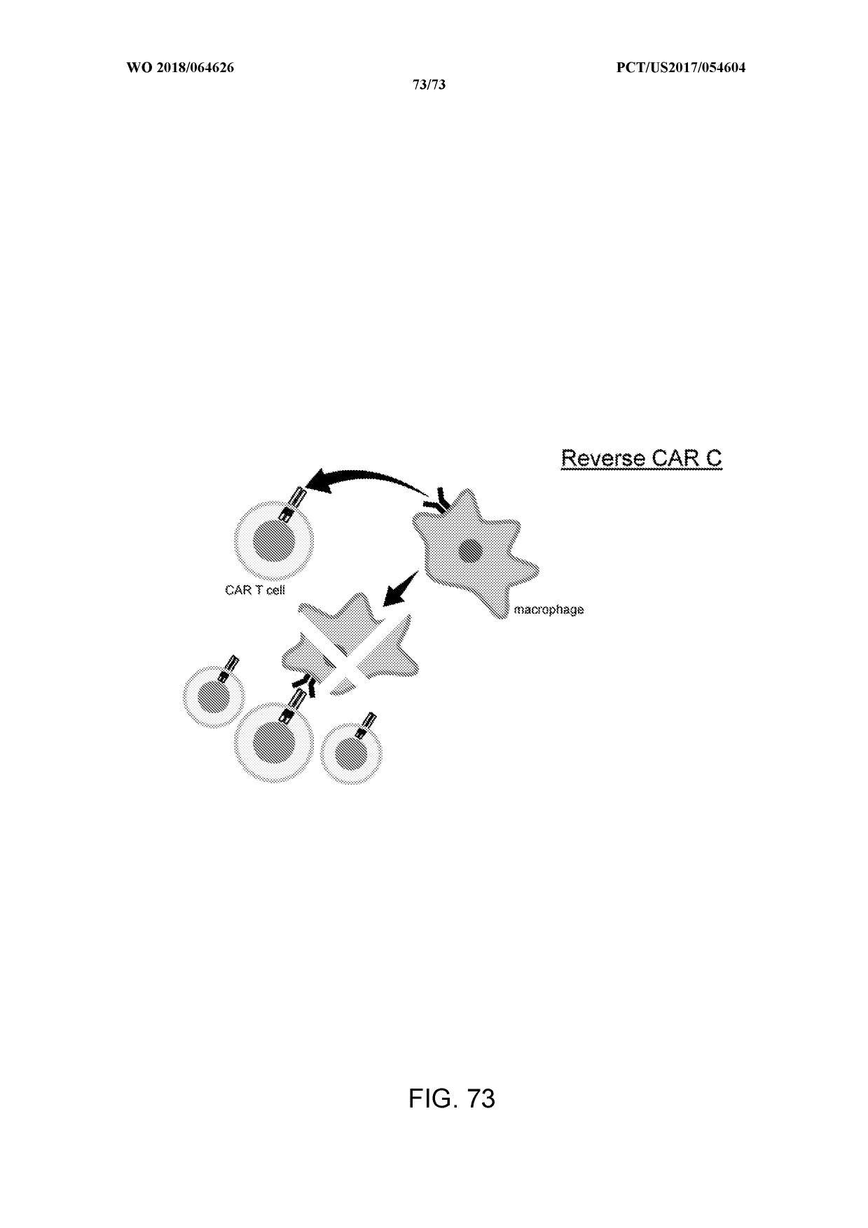

In FIG. 73, there is another embodiment of the reverse CAR. In this embodiment, immune cells, such as T cells, express a molecule (for example, a chimeric Fc receptor target molecule) that can be recognized by macrophages (as an example, CH2CH3) while the endodomains comprise a costimulatory domain (for example, CD28) and CD3zeta. In this embodiment, once T cells expressing the chimeric Fc receptor target molecule get recognized by macrophages, this will induce: (i) killing of macrophages by activation of CD3zeta and, (ii) T cell proliferation by activation of the costimulatory domain (such as CD28).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of producing an engineered chimeric receptor having a spacer and an antigen recognition domain, said method comprising the step of evaluating tonic signaling in cells expressing the receptor.

2. The method of claim 1, further comprising the step of evaluating antigen recognition for the receptor.

3. The method of claim 1, wherein following one or both of the evaluating steps, at least part of the chimeric receptor is modified.

4. The method of claim 3, wherein the part of the chimeric receptor that is modified is the spacer, the antigen recognition domain, an exodomain comprising the antigen recognition domain or part thereof, a transmembrane domain, and/or an endodomain or part thereof.

5. The method of claim 4, wherein the spacer is modified.

6. The method of claim 1, wherein tonic signaling is evaluated by one or more of the following:
a) measuring metabolic activity of the cells;
b) measuring one or more indicators of cell activation in the absence of stimulation by an antigen recognized by the receptor;
c) measuring one or more phenotypical changes related to cell aging or cell senescence;
d) determining cell cycle progression in the absence of antigenic stimulation; and
e) measuring cell size of cells expressing the receptor compared to the size of unmodified cells.

7. The method of claim 1, wherein tonic signaling is evaluated by one or more of the following:
a) measuring metabolic activity of the cells in the absence of antigenic stimulation and compared to unmodified cells and/or a control vector without tonic signaling;
b) measuring one or more indicators of cell activation in the absence of antigenic stimulation and compared to unmodified cells and/or a control vector without tonic signaling;
c) measuring one or more phenotypical changes related to cell aging or cell senescence in the absence of antigenic stimulation and compared to unmodified cell and/or a control vector without tonic signaling;
d) determining cell cycle progression in the absence of antigenic stimulation and compared to unmodified cells and/or a control vector without tonic signaling;
e) measuring cell size of cells expressing the receptor in the absence of antigenic stimulation and compared to unmodified cells and/or a control vector without tonic signaling; and
f) measuring the cytokine production of cells in the absence of antigenic stimulation and compared to unmodified cells and/or a control vector without tonic signaling.

8. The method of claim 1, wherein antigen recognition by said antigen recognition domain is evaluated by one or more of the following:
a) the efficacy of the binding of the antigen recognition domain to an antigen;
b) an in vitro killing assay of one or more cells expressing the receptor;
c) an in vivo assay measuring tumor size or burden following delivery of cells expressing the receptor;
d) cytokine production of one or more cells expressing the receptor;
e) the in vivo proliferation of one or more cells that express the receptor; and
f) antitumor activity of immune cells expressing the receptor.

9. The method of claim 1, wherein the tonic signaling is evaluated by a) phenotype of cells expressing the receptor, b) growth pattern of cells expressing the receptor in the absence of the antigen in comparison to growth pattern of non-transduced cells and/or a control vector without tonic signaling.

10. The method of claim 8, wherein when the T cell phenotype of cells expressing the receptor approximate the content of naïve and central memory cells of non-transduced cells and/or a control vector in the absence of antigen stimulation, the cells will have low tonic signaling.

11. The method of claim 8, wherein when the T cell phenotype comprises a similar content (±10%) of naïve and central memory cells among cells expressing the receptor compare to non-transduced cells and/or a control vector, it is considered to have a low tonic signal.

12. The method of claim 8, wherein two different configuration of receptors are compared in the content of CCR7+ after about two weeks in the absence of the antigen, and wherein the configuration having greater CCR7 is a configuration with the lower tonic signal.

13. The method of claim 8, wherein when at least 30% of cells expressing the receptor are CCR7+ after about two weeks in absence of the antigen, the cells are predicted to have low tonic signaling.

14. The method of claim 8, wherein among cells expressing the receptor, when the amount of CCR7+ cells is similar to the number of CCR7+ non-transduced cells under the same culture conditions, and/or control construct under the same culture conditions, the cells are predicted to have low tonic signaling.

15. The method of claim 8, wherein when the growth pattern of cells expressing the receptor is similar to non-transduced T cells and/or control construct in the absence of the antigen, are predicted to have low tonic signaling.

16. The method of claim 6, wherein metabolic activity is measured within 2 to 3 days after transduction of the cells with a polynucleotide encoding the receptor.

17. The method of claim 6, wherein the metabolic activity is determined by the level of glucose produced by the cell, the level of lactate produced by the cell, or a ratio thereof.

18. The method of claim 1, wherein the one or more indicators of cell activation comprise the level of CD25, CD69, 41BB, CD71, CD40, and/or HLADR.

19. The method of claim 1, wherein the one or more indicators of cell activation comprise the level of one or more cytokines produced by the cells.

20. The method of claim 19, wherein the cytokine is interferon gamma, TNF, IL2, INFb, GMCSF, IL6, IL8, perforin, IL13, IL4, TGFb, or a combination thereof.

21. The method of claim 6, wherein when the receptor comprises the CD3 zeta chain, the one or more indicators of cell activation comprises the phosphorylation of the CD3 zeta chain in the absence of antigenic stimulation.

22. The method of claim 7, wherein the cytokine production comprises production of interferon gamma, IL2, TNF, INFb, GMCSF, perforin, IL6, IL8, IL13, IL4, TGFb, or a combination thereof.

23. The method of claim 1, wherein the spacer length and/or content is selected for the purpose of said evaluating.

24. The method of claim 1, wherein when an epitope on an antigen to which the receptor recognizes is proximal to the cell membrane, a spacer that is >150 amino acids is selected.

25. The method of claim 1, wherein when an epitope on an antigen to which the receptor recognizes is exposed or distant to the antigen, a spacer that is <50 amino acids is selected.

26. The method of claim 1, wherein the spacer is derived from IgG2.

27. The method of claim 26, wherein the spacer comprises CH2 and CH3 from IgG2.

28. The method of claim 1, wherein the spacer comprises the hinge from IgG2.

29. The method of claim 1, wherein the spacer comprises CH3 from IgG2.

30. The method of claim 1, wherein the spacer lacks CH2 from IgG2.

31. The method of claim 1, wherein the spacer comprises one or more modifications to reduce binding of the spacer to an Fcγ receptor.

32. A polynucleotide encoding the engineered receptor produced by the method of claim 1.

33. The polynucleotide of claim 32, wherein said polynucleotide is comprised in a vector.

34. The polynucleotide of claim 33, wherein the vector is comprised in a cell.

35. The polynucleotide of claim 34, wherein the cell is an immune cell.

36. A chimeric antigen receptor encoded by the polynucleotide of claim 32.

37. A chimeric antigen receptor produced by the method of claim 1.

38. A pharmaceutical composition comprising the chimeric antigen receptor of claim 36.

39. A cell expressing the polynucleotide of claim 30.

40. A method of targeting a Fc-gamma receptor (FcγR)-bearing cell, comprising the step of exposing to the FcγR-bearing cell an immune cell that expresses a chimeric Fc receptor target molecule that comprises one or more FcγR-binding domains of an IgG Fc domain, wherein the exposing is deliberately performed to target the FcγR-bearing cell.

41. The method of claim 40, wherein the FcγR-binding domain comprises the CH2CH3 region, the CH2 region, and/or the CH3 region of an IgG.

42. The method of claim 41, wherein the CH2CH3 region, the CH2 region, and/or the CH3 region is from IgG1, IgG2, or IgG4.

43. The method of claim 40, wherein the chimeric Fc receptor target molecule further comprises CD3 zeta-chain of the TCR/CD3 complex and wherein the FcγR-bearing cell is killed.

44. The method of claim 40, wherein the chimeric Fc receptor target molecule further comprises a scFv.

45. The method of claim 25, wherein the chimeric Fc receptor target molecule lacks the CD3 zeta-chain of the TCR/CD3 complex.

46. The method of claim 40, wherein the chimeric FC receptor target molecule comprises one or more costimulatory domains.

47. The method of claim 46, wherein the one or more costimulatory domains are selected from the group consisting of CD28, OX40, 4-1BB, ICOS, CD27, CD95, CD43, KLRG1, CD4OL, CD137, CD137L, CD134, CD30, and a combination thereof.

48. The method of claim 40, wherein the immune cell is a T cell, NK cell, NKT cell, B cells, monocytes, macrophages, or dendritic cells.

49. The method of claim 40, wherein the FcγR-bearing cell is a monocyte, macrophage, dendritic cell, neutrophil, eosinophils, platelets (RIIa), B cells (RIIb), or NK (RIIc and RIIIa).

50. The method of claim 40, wherein the method occurs in vivo in an individual that has a medical condition with chronic inflammation as a symptom.

51. The method of claim 50, wherein the medical condition with chronic inflammation is arthritis, multiple sclerosis, diabetic ulcers, atherosclerosis, asthma, sepsis, cardiovascular disease, or Alzheimer's Disease.

52. The method of claim 38, wherein the method occurs in vivo in an individual that has cancer, arthritis, multiple sclerosis, diabetic ulcers, atherosclerosis, asthma, sepsis, cardiovascular disease, or Alzheimer's Disease.

53. A method of treating an individual having cancer, comprising administering to the individual a therapeutically effective amount of the chimeric antigen receptor of claim 36, wherein the cancer expresses a tumor-associated antigen or tumor-specific antigen, and the chimeric antigen receptor is targeted to the tumor-associated antigen or tumor-specific antigen.

54. A method of selecting a chimeric antigen receptor having a spacer between an antigen recognition domain and a transmembrane domain, comprising the steps of (a) expressing a first chimeric antigen receptor in a type of immune cell and determining a first level of tonic signaling in the immune cell; (b) subsequently expressing a second chimeric antigen receptor having a longer or shorter spacer; (c) expressing the chimeric antigen receptor having said longer or shorter spacer in said type of immune cell, and determining a second level of tonic signaling in the immune cell; wherein if said second level is lower than said first level, said second chimeric antigen receptor is selected, and if said first level is lower than said first level, said first chimeric antigen receptor is selected.

55. The method of claim 1, comprising repeating steps (a)-(c) for a plurality of times with chimeric antigen receptors having spacers of a different length for each of said plurality of times, and selecting said chimeric antigen receptor that is expressed by the immune cell determined to have the least tonic signaling.
